(19) United States
(12) Patent Application Publication
(10) Pub. No.: US 2020/0332376 A1
(43) Pub. Date: Oct. 22, 2020

(54) ECONOMICAL METHODS FOR PERFORMING OXIDATIVE CATALYTIC PRETREATMENT OF PLANT BIOMASS USING A HOMOGENEOUS CATALYST SYSTEM

(71) Applicants: Board of Trustees of Michigan State University, East Lansing, MI (US); Montana State University, Bozeman, MT (US); Wisconsin Alumni Research Foundation, Madison, WI (US)

(72) Inventors: Eric L. Hegg, East Lansing, MI (US); Zhaoyang Yuan, Lansing, MI (US); David B. Hodge, Bozeman, MT (US); Shannon S. Stahl, Madison, WI (US); Bryan D. Bals, Indianapolis, IN (US)

(21) Appl. No.: 16/903,598

(22) Filed: Jun. 17, 2020

Related U.S. Application Data
(63) Continuation-in-part of application No. 16/206,848, filed on Nov. 30, 2018.
Application No. 16/206,848 is a division of application No. 14/729,756, filed on Jun. 3, 2015, now abandoned.
(60) Provisional application No. 63/025,502, filed on May 15, 2020.
(60) Provisional application No. 62/007,306, filed on Jun. 3, 2014.

(51) Int. Cl. C13K 13/00; C07F 1/08; C07F 13/00; B01J 31/18; D21B 1/02; C13K 1/02; B01J 31/22

(52) U.S. Cl. CPC C13K 13/002; B01J 2531/845; C07F 13/005; B01J 31/1805; D21B 1/021; C13K 1/02; B01J 31/1815; B01J 31/2243; B01J 31/183; B01J 2531/16; C12P 2201/00; B01J 2231/70; B01J 2531/842; B01J 2531/72; C07F 1/08

(57) ABSTRACT

An improved alkaline pretreatment of biomass is provided that uses a homogenous catalyst with one or more metals and metal coordinating ligands, wherein the homogeneous catalyst is used with at least two oxidants in an oxidation reaction to catalytically pretreat lignocellulosic biomass. In one embodiment, hydrogen peroxide and oxygen are utilized as co-oxidants during alkaline-oxidative pretreatment to improve biomass pretreatment and increase enzymatic digestibility. In one embodiment, the homogenous catalyst is copper (II) 2,2′-bipyridine (Cu(bpy)). Related methods are also disclosed to improve the economic feasibility of production of lignocellulose derived sugars.

The present application claims benefit under 35 U.S.C. 119(e) of U.S. Provisional Application Ser. No. 63/025,502, filed on May 15, 2020, which application is hereby incorporated by reference in its entirety. This application is also a Continuation-in-Part of U.S. patent application Ser. No. 16/206,848 filed on Nov. 30, 2018 and published as U.S. Publication No. 2019/0091674 A1 on Mar. 28, 2019, which application is a divisional of U.S. patent application Ser. No. 14/729,756 filed on Jun. 3, 2015, now abandoned, which claims benefit under 35 U.S.C. 119(e) of U.S. Provisional Application Ser. No. 62/007,306 filed on Jun. 3, 2014, now abandoned, which applications are all hereby incorporated by reference in their entireties.

### STATEMENT OF GOVERNMENT RIGHTS

This invention was made with government support under DE-EE0008148 awarded by the U.S. Department of Energy. The government has certain rights in the invention.

### BACKGROUND

Cellulosic biofuels offer enormous potential as sustainable, low-carbon alternative liquid transportation fuels to petroleum-derived fuels. The vast majority of carbon in the terrestrial biosphere is contained in the cell walls of plants or lignocellulose. This enormous reservoir of reduced carbon is largely untapped for conversion to fuels, chemicals, and polymers as consequence of the difficulty in deconstructing the biopolymers contained in the plant cell wall matrix to a suite of chemicals that are amenable to conversion processes.

### SUMMARY

Concerns over energy security and climate change have resulted in considerable effort focused on facilitating the transition of the fossil fuel-based economy to a renewable resource-based sustainable economy. Among the renewable resources currently available, lignocellulosic biomass is an abundant, potentially carbon-neutral source and is considered to be a promising raw material for the production of biofuels and biomaterials at large scales. The challenge of developing efficient lignocellulosic biorefineries, however, is the high recalcitrance and complex structure of the plant cell walls which hinders both bioconversion and chemical conversion processes. Thus, a pretreatment technology that can economically fractionate lignocellulosic biomass into renewable biopolymers is highly desirable. The fractionated polysaccharides can be converted into monomeric sugars and biofuels, while lignin can be used as the starting material for the production of various chemicals, materials, and fuels. Thus, an effective pretreatment approach that simultaneously maximizes polysaccharide hydrolysis to fermentable sugars and extracts high-quality lignin for subsequent valorization would improve the economic viability of the lignocellulosic-to-biofuel industry.

A sequential two-stage pretreatment process includes an alkaline pre-extraction stage followed by an alkaline oxidative pretreatment process with a metal-ligand homogenous catalyst. A challenge associated with this two-stage alkaline pre-extraction/Cu-catalyzed alkaline hydrogen peroxide (Cu-AHP) pretreatment process as well as other oxidative pretreatment processes is the process economics, especially the cost of the hydrogen peroxide (H₂O₂). To improve the economics of this oxidative lignocellulosic-to-biofuel technique, a reduction in the loading of expensive chemical inputs in conjunction with an increase in the recovery of biomass polymers is advantageous.

Employing O₂, e.g. at about 50 psi, in addition to H₂O₂ as a co-oxidant during a two-stage alkaline pre-extraction/copper 2,2′-bipyridine catalyzed alkaline hydrogen peroxide (Cu-AHP) pretreatment process resulted in a substantial improvement in delignification relative to using H₂O₂ alone during the second-stage Cu-AHP pretreatment, leading to high overall sugar yields even at H₂O₂ loadings as low as 2% (w/w of original biomass). The Cu-AHP pretreatment process while employing oxygen will be referred to herein as Cu-AHP/O process. The presence of H₂O₂, however, was critical. Performing analogous reactions in the absence of H₂O₂ resulted in approximately 25% less delignification and a 30% decrease in overall sugar yields. The isolated lignin from this dual oxidant process had high aliphatic hydroxyl group content and reactivity to isocyanate, indicating that it is a promising substrate for the production of polyurethanes. To test the suitability of the isolated lignin as a source of aromatic monomers, the lignin was subjected to a sequential Bobbitt's salt oxidation followed by formic-acid catalyzed depolymerization process. Monomer yields of approximately 17% were obtained, and the difference in yields was not significant between lignin isolated from the Cu-AHP process with and without O₂ as a co-oxidant, suggesting that the addition of O₂ did not lead to significant lignin crosslinking. Technoeconomic analysis (TEA) indicates that using both O₂ and H₂O₂ as co-oxidants for the Cu-AHP/O alkaline oxidative pretreatment process could potentially reduce the minimum biofuel selling price (MFSP) to as low as $0.77/L, 40% lower than using H₂O₂ only.

The present description includes a process for improving the economics by utilizing a co-oxidant that can partially or completely substitute for the H₂O₂ while maintaining or increasing monomer sugar yields and lignin solubilization. The present description describes using O₂ as a co-oxidant along with H₂O₂ during the two-stage alkaline pre-extraction/Cu-AHP/O pretreatment process to improve the recovery of biopolymers, including both polysaccharides and lignin. The results indicate that the inclusion of O₂ as a co-oxidant led to enhanced lignin solubilization, a lower chemical input requirement, improved total sugar release by enzymatic hydrolysis, and reduced processing costs. Moreover, the properties of the lignin isolated from this process (high hydroxyl content, >6.5 mmol/g) indicate that it could be an excellent renewable polyol in the formulation of lignin-based polyurethanes. Furthermore, the susceptibility of the lignin towards oxidative depolymerization suggests that the use of O₂ as a co-oxidant did not result in the formation of additional lignin crosslinks, and the lignin remains amenable to valorization via depolymerization into monomers. Technoeconomic analysis (TEA) suggests that minimum fuel selling price (MFSP) of the O₂-enhanced Cu-AHP (Cu-AHP/O) pretreatment could be reduced by as much as 40% relative to using H₂O₂ alone. Together, these results highlight the utility of using O₂ as an inexpensive and environmentally friendly co-oxidant in conjunction with H₂O₂ during metal-catalyzed alkaline oxidative pretreatments.

In the following detailed description, embodiments are described in sufficient detail to enable those skilled in the art to practice them, and it is to be understood that other embodiments may be used and that chemical and procedural changes may be made without departing from the spirit and scope of the present subject matter. The following detailed description is, therefore, not to be taken in a limiting sense, and the scope of embodiments of the present invention is defined only by the appended claims.

The various embodiments will be further described by reference to the following examples, which are offered to further illustrate various embodiments. It should be understood, however, that many variations and modifications may be made while remaining within the scope of the various embodiments.

The various embodiments described herein provide a homogeneous catalyst system comprising one or more metals; at least one metal coordinating ligand; and at least two oxidants, wherein the homogeneous catalyst system is a metal-ligand complex configured to catalytically delignify lignocellulosic biomass with the at least two oxidants in an oxidative pretreatment reaction.

The various embodiments described herein provide a method of pretreating plant biomass comprising catalytically pretreating the plant biomass with a metal-ligand complex and at least two oxidants in an alkaline oxidative pretreatment process to produce a catalytically pretreated plant biomass. In one embodiment, the oxidants are oxygen and hydrogen peroxide. In one embodiment, the oxygen pressure is from about 25 psi to about 100 psi and the hydrogen peroxide is less than about 5% (w/w of original biomass). In one embodiment, at least two oxidants are present simultaneously in the pretreatment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing the effect of metal catalyst addition to a 24 hour (hr) alkaline oxidative pretreatment (AOP) of hybrid poplar according to an embodiment.

FIG. 2 is a graph showing enzymatic digestibility of pretreated poplar according to an embodiment.

FIG. 3 is a graph showing use of recovered catalyst in catalytic alkaline oxidative pretreatment (AOP) according to an embodiment.

FIG. 4A is a graph showing phenolic acid and aldehyde yields during various pretreatments of hybrid poplar according to an embodiment.

FIG. 4B is a graph showing p-hydroxybenzoate yield during various pretreatments of hybrid poplar according to an embodiment.

FIG. 5 is a graph showing the effect of a catalyst in PTL for oxidative pretreatment on enzymatic digestibility of pretreated biomass according to an embodiment.

FIG. 6 is a graph showing yields of glucose and xylose (with copper recycling) under varying pretreatment conditions according to various embodiments.

FIG. 7 is a graph showing glucose and xylose yield (with extractives removal) under varying conditions according to various embodiments.

FIG. 8 is a graph showing glucose and xylose yield (with NaOH recycle) under varying conditions according to various embodiments.

FIGS. 9A-9C are Transmission Electron Microscopy (TEM) micrographs of cross sections of an untreated (A) and Alkaline Oxidative Peroxide (AOP)-only pretreated (B, C) hybrid poplar cell wall according to various embodiments.

FIGS. 10A-10D are Transmission Electron Microscopy (TEM) micrographs of a hybrid poplar cell wall after copper(II) 2,2′-bipyridine complex (Cu(bpy))-catalyzed alkaline oxidative pretreatment (Cu-AOP) showing delamination (A) and dislocations of cell wall layers (B), together with accumulation of nanoparticles in disrupted regions (C, D) according to various embodiments.

FIGS. 11A-11F are Transmission Electron Microscopy (TEM) micrographs of hybrid poplar cell wall (A) and high resolution image of an electron-opaque aggregate (B) together with acquired Energy Dispersive X-ray Spectroscopy (EDS) spectra of select regions within this sample (C—F) according to various embodiments.

FIG. 12 shows SEC elution profiles for plant cell wall polymers solubilized during pretreatments referenced to elution times for polystyrene standards according to various embodiments.

FIGS. 13A-13F are partial 2D Heteronuclear Single-Quantum Coherence (HSQC) Nuclear Magnetic Resonance (NMR) spectra of (A, B) whole cell wall untreated poplar, (C, D) solubilized lignin, and (E, F) residual poplar cell walls following Cu-catalyzed alkaline oxidative pretreatment (Cu-AOP) showing polysaccharide correlations and colored contours to match structures for aromatic components according to various embodiments.

FIGS. 14A-14L are representations of various known chemical structures.

FIG. 15 is an Electron Energy Loss Spectroscopy (EELS) spectrum of the Cu-containing nanoparticles showing the Cu L_{2,3} edge providing evidence that the Cu in these particles is primarily in the Cu(I) oxidation state with contributions by Cu(0) according to various embodiments.

FIG. 16 is a graph comparing yields of xylose and glucose when using catalytic alkaline oxidative pretreatment (AOP) with a batch addition of hydrogen peroxide versus a slow addition of hydrogen peroxide according to an embodiment.

FIG. 17 is a graph comparing yields of xylose and glucose yields when using catalytic AOP with a batch addition of hydrogen peroxide versus a slow addition of hydrogen peroxide in combination with alkali pre-extraction in a modified Cu-alkaline oxidative pretreatment (Cu-AOP) according to an embodiment.

FIG. 18 is a graph showing the correlation between lignin removal and glucose yields with different concentrations of slowly added H₂O₂ in combination with alkali pre-extraction in a modified Cu-alkaline oxidative pretreatment (Cu-AOP) according to an embodiment.

FIG. 19 is a graph showing glucose yields obtained with different concentrations of enzymes at different loadings of H₂O₂ on biomass utilizing a modified Cu-alkaline oxidative pretreatment (Cu-AOP) according to an embodiment.

FIG. 20 is a graph showing glucose yields obtained with different concentrations of bipyridine at different loadings of H₂O₂ and enzymes on biomass utilizing modified Cu-alkaline oxidative pretreatment (Cu-AOP) according to an embodiment.

FIG. 21 is an image of lignin after the biomass in which it was located was subject to a Cu-AOP pretreatment according to an embodiment.

FIGS. 22A-22C are graphs correlating sugar yields with different cell wall properties according to an embodiment.

FIG. 23A is a graph showing metal content of various genotypes of Populus trichocarpa species according to an embodiment.

FIG. 23B is a graph showing lignin content vs guaiacyl and syringyl (S/G) ratio according to an embodiment.

FIG. 24 is a graph showing glucose yields for various chelated and non-chelated for four different genotypes of Populus trichocarpa according to an embodiment.

FIG. 25 is a graph showing total number of cell wall redox metal ions in various chelated and non-chelated for four different genotypes of Populus trichocarpa according to an embodiment.

FIG. 26 is a graph showing glucose yields for various hardwood species after undergoing different pretreatments according to various embodiments.

FIG. 27 is a graph showing glucose yield to S/G ratio for various types of hardwood species after undergoing various types of pretreatment processes according to various embodiments.

FIG. 28 is a graph showing glucose yield correlation to cell wall redox active metal content for Copper (Cu), Manganese (Mn) and iron (Fe) after undergoing a pretreatment process according to various embodiments.

FIGS. 29A-29B is a graph showing glucose yields to S/G ratio for various hardwood species after undergoing an alkaline oxidative pretreatment (AOP)-chelated and alkaline oxidative pretreatment (AOP)-non-chelated pretreatment according to various embodiments.

FIG. 30 is a graph showing glucose yields for various hardwood species after undergoing different pretreatments according to various embodiments.

FIG. 31 is a graph showing solubilized lignin and xylan from poplar following the second-stage alkaline-oxidative pretreatment according to various embodiments.

FIGS. 32A-32B are graphs showing impact of alkaline-oxidative pretreatment conditions on overall sugar yields following enzymatic hydrolysis of hybrid poplar according to various embodiments.

FIGS. 33A-33B are graphs showing the impact of the second-stage Cu-alkaline oxidative pretreatment (Cu-AOP) conditions on (FIG. 33A) the hydroxyl group content and (FIG. 33B) the reactivity towards isocyanate (MDI) of the lignin samples according to various embodiments.

FIG. 34 is a graph of monomer yields (on a lignin basis) from a sequential oxidation and depolymerization of lignin isolated using different alkaline-oxidative pretreatment conditions according to various embodiments.

FIGS. 35A-35B are graphs showing pretreatment operating cost (FIG. 35A) in million $/year and minimum fuel selling price (MFSP) (FIG. 35B) in $/L for various Cu-alkaline oxidative pretreatment (Cu-AOP) conditions according to various embodiments.

FIG. 36 is a graph correlating overall glucose yields following enzymatic hydrolysis with extent of lignin removal according to an embodiment.

### DETAILED DESCRIPTION OF THE EMBODIMENTS

In the following detailed description, embodiments are described in sufficient detail to enable those skilled in the art to practice them, and it is to be understood that other embodiments may be used and that chemical and procedural changes may be made without departing from the spirit and scope of the present subject matter. The following detailed description is, therefore, not to be taken in a limiting sense, and the scope of embodiments of the present invention is defined only by the appended claims.

The term “biomass” as used herein, refers in general to organic matter harvested or collected from a renewable biological resource as a source of energy and bioproducts. The renewable biological resource can include plant materials, animal materials, and/or materials produced biologically. The term “biomass” is not considered to include fossil fuels, which are not renewable.

The term “plant biomass” or “ligno-cellulosic biomass (LCB)” as used herein is intended to refer to virtually any plant-derived organic matter containing cellulose and/or hemicellulose as its primary carbohydrates (woody or non-woody) available for producing energy on a renewable basis and bioproducts. Plant biomass can include, but is not limited to, agricultural residues such as corn stover, wheat straw, rice straw, sugar cane bagasse, Sorghum, and the like. Plant biomass can also include agricultural residues and forest residues that are dedicated for bioenergy purposes, such as residues of grasses and trees. Plant biomass further includes, but is not limited to, “woody biomass”, i.e., woody energy crops, wood wastes and residues such as trees, including fruit trees, such as fruit-bearing trees, (e.g., apple trees, orange trees, and the like), softwood forest thinnings, barky wastes, sawdust, paper and pulp industry waste streams, wood fiber, and the like. Additionally, perennial grass crops, such as various prairie grasses, including prairie cord grass, switchgrass, Miscanthus, big bluestem, little bluestem, side oats grama, and the like, have potential to be produced large-scale as additional plant biomass sources. For urban areas, potential plant biomass feedstock includes yard waste (e.g., grass clippings, leaves, tree clippings, brush, etc.) and vegetable processing waste. Plant biomass is known to be the most prevalent form of carbohydrate available in nature and corn stover is currently the largest source of readily available plant biomass in the United States. When describing the various embodiments and used without a qualifier, the term “biomass” is intended to refer to “plant biomass,” i.e., lignocellulosic biomass (LCB) containing plant cell wall polysaccharides.

The term “biofuel” as used herein, refers to any renewable solid, liquid or gaseous fuel produced biologically and/or chemically, for example, those derived from biomass. Most biofuels are originally derived from biological processes such as the photosynthesis process and can therefore be considered a solar or chemical energy source. Some types of biofuels, such as some types of biodiesel, can be derived from animal fats. Other biofuels, such as natural polymers (e.g., chitin or certain sources of microbial cellulose), are not synthesized during photosynthesis, but can nonetheless be considered a biofuel because they are biodegradable. There are generally considered to be three types of biofuels derived from biomass synthesized during photosynthesis, namely, agricultural biofuels (defined below), municipal solid waste biofuels (residential and light commercial garbage or refuse, with most of the recyclable materials such as glass and metal removed) and forestry biofuels (e.g., trees, waste or byproduct streams from wood products, wood fiber, pulp and paper industries). Biofuels produced from biomass not synthesized during photosynthesis include, but are not limited to, those derived from chitin, which is a chemically modified form of cellulose known as an N-acetyl glucosamine polymer. Chitin is a significant component of the waste produced by the aquaculture industry because it comprises the shells of seafood.

The term “agricultural biofuel”, as used herein, refers to a biofuel derived from agricultural crops, lignocellulosic crop residues, grain processing facility wastes (e.g., wheat/oat hulls, corn/bean fines, out-of-specification materials, etc.), livestock production facility waste (e.g., manure, carcasses, etc.), livestock processing facility waste (e.g., undesirable parts, cleansing streams, contaminated materials, etc.), food processing facility waste (e.g., separated waste streams such as grease, fat, stems, shells, intermediate process residue, rinse/cleansing streams, etc.), value-added agricultural facility byproducts (e.g., distiller's wet grain (DWG) and syrup from ethanol production facilities, etc.), and the like. Examples of livestock industries include, but are not limited to, beef, pork, turkey, chicken, egg and dairy facilities. Examples of agricultural crops include, but are not limited to, any type of non-woody plant (e.g., cotton), grains such as corn, wheat, soybeans, Sorghum, barley, oats, rye, and the like, herbs (e.g., peanuts), short rotation herbaceous crops such as switchgrass, alfalfa, and so forth.

The term “pretreatment step” as used herein, refers to any step intended to alternative biomass so it can be more efficiently and economically converted to reactive intermediate chemical compounds such as sugars, organic acids, etc., which can then be further processed to a variety of end products such as ethanol, isobutanol, long chain alkanes etc. Pretreatment can reduce the degree of crystallinity of a polymeric substrate, reduce the interference of lignin with biomass conversion, and hydrolyze some of the structural carbohydrates, thus increasing their enzymatic digestibility and accelerating the degradation of biomass to useful products. Pretreatment methods can utilize acids of varying concentrations, including dilute acid pretreatments, concentrated acid pretreatments (using, for example, sulfuric acids, hydrochloric acids, organic acids, and the like) and/or pretreatments with alkali such as ammonia and/or ammonium hydroxide and/or calcium hydroxide and/or sodium hydroxide and/or lime, and the like, and/or oxidative pretreatments using oxidants such as air, oxygen, hydrogen peroxide, organic peroxide, ozone, and the like.

Pretreatment methods can additionally or alternatively utilize hydrothermal treatments including water, heat, steam or pressurized steam pretreatments, including, but not limited to, hydro-thermolysis pretreatment and liquid hot water pretreatment, further including, for example, acid catalyzed steam explosion pretreatment (e.g., SO₂ catalyzed). Pretreatment can occur or be deployed in various types of containers, reactors (e.g., batch, counter-current, and the like), pipes, flow through cells and the like. Many pretreatment methods will cause the partial or full solubilization and/or destabilization of lignin and/or hydrolysis of hemicellulose to pentose sugars. Further examples of pretreatment include, but are not limited wet oxidation, organosolv pretreatment and mechanical extrusion.

The term “alkaline oxidative pretreatment (AOP)” as used herein refers to a pretreatment process in which plant biomass is pretreated under alkaline conditions using one or more oxidative chemicals, which can include, but are not limited to, hydrogen peroxide, oxygen, ozone, hydroperoxide anion, superoxide radical, hydroxyl radical, and peroxy acids (e.g., peracetic acid, peroxymonosulfuric acid, peroxyphosphoric acid, meta-chloroperoxybenzoic acid). See, for example, Liu, et al., Coupling alkaline pre-extraction with alkaline-oxidative post-treatment of corn stover to enhance enzymatic hydrolysis and fermentability, Biotechnology for Biofuels, 2014, 7:48, which describes example conditions for alkaline oxidative pretreatment. An alkaline oxidative pretreatment which uses hydrogen peroxide as one of the oxidants is to be distinguished from a conventional “alkaline hydrogen peroxide” (AHP) pretreatment which is a one-step catalytic pretreatment process which requires much higher oxidant loadings. See, for example, Biotechnol Bioeng 1984, 26:46-52; Biotechnol Bioeng 1984, 26:628-631; Biotechnol Bioeng 1985, 27:225-231; Science 1985, 230:820-822 and Biotechnol Biofuels 2011, 4:16, which describe conventional AHP with much higher oxidant loadings.

The term “metal-ligand complex” as used herein refers to a metal complex containing one or more metal-coordinating ligands and one or more metal atoms which are in a state of interaction with each other. Such interactions include various types of forces and bonds, which include, but are not limited to, ionic bonds, covalent bonds, and van der Waals forces. The term “metal-ligand complex” and “ligand-metal complex” can be used interchangeably.

The term “metal-coordinating ligand” as used herein refers to a ligand, such as an ion, a molecule, or the like, that is capable of interacting with the metal portion of a metal-ligand complex. When used without qualification, the term “ligand” is intended to refer to a “metal-coordinating ligand.”

The term “copper-coordinating ligand” as used herein refers to a metal coordinating ligand capable of interacting with copper atoms or copper ions.

The term “single-ligand metal complex” as used herein refers to a metal-ligand complex containing only one ligand that coordinates with, i.e., interacts with metal atoms or metal ions.

The term “multi-ligand metal complex” as used herein refers to a metal-ligand complex containing more than one ligand that coordinates with, i.e., interacts with metal atom or metal ions.

The term “toxicity” as used herein refers to ions, molecules and metal-ligand complexes present in the process streams during biomass conversion and cellulosic biofuel production that negatively impact the yield of the products.

The term “slow add” as used herein refers to a gradual rate of addition of a reagent to a reaction vessel. The gradual rate can be continuous or discontinuous, i.e., can include intermittent periods of no reagent being added. A “slow add” is in contrast to a batch method of adding a reagent, in which all the desired reagent is added to the reactive vessel at once.

The term “state of interaction” as used herein refers to an interaction between a ligand and a metal or between a metal and multiple ligands. Such an interaction can include various types of forces and bonds, which include, but are not limited to, ionic bonds, covalent bonds, and van der Waals forces.

Nearly all forms of lignocellulosic biomass, i.e., plant biomass, such as monocots, comprise three primary chemical fractions: hemicellulose, cellulose, and lignin. Lignin, which is a polymer of phenolic molecules, provides structural integrity to plants, and is difficult to hydrolyze. As such, after sugars in the biomass have been fermented to a bioproduct, such as alcohol, lignin remains as residual material, i.e., a non-easily digestible portion.

Cellulosic biofuel production from lignocellulosic biomass has gained considerable momentum due to both environmental and social sustainability benefits. However, the technology is not yet fully commercialized. One issue impeding cellulosic biofuel production using the sugar platform is the hydrolysis-resistant nature of certain components in the lignocellulosic biomass.

Cellulose and hemicelluloses in plant cell walls exist in complex structures within the residual material. Hemicellulose is a polymer of short, highly branched chains of mostly five-carbon pentose sugars (xylose and arabinose), and to a lesser extent six-carbon hexose sugars (galactose, glucose and mannose). Because of its branched structure, hemicellulose is amorphous and relatively easy to hydrolyze into its individual constituent sugars by enzyme or dilute acid treatment. Cellulose is a linear polymer comprising of β(1→4) linked D-glucose in plant cell wall, much like starch with a linear/branched polymer comprising of α(1→4) linked D-glucose, which is the primary substrate of corn grain in dry grind and wet mill ethanol plants. However, unlike starch, the glucose sugars of cellulose are strung together by β-glycosidic linkages, which allow cellulose to form closely associated linear chains. Because of the high degree of hydrogen bonding that can occur between cellulose chains, cellulose forms a rigid crystalline structure that is highly stable and much more resistant to hydrolysis by chemical or enzymatic attack than starch or hemicellulose polymers. Although hemicellulose sugars represent the “low-hanging” fruit for conversion to a biofuel, the substantially higher content of cellulose represents the greater potential for maximizing biofuel yields, on a per ton basis of plant biomass.

Lignocellulose can also be characterized as a highly heterogeneous composite material comprised of multiple cell wall biopolymers (cellulose, heteropolysaccharides including hemicelluloses and pectins, and lignins) associated primarily by non-covalent interactions which are assembled into cell walls with composition and properties varying by cell and tissue type. These components are interconnected through a variety of covalent and non-covalent interactions, giving rise to a highly organized network which is assembled in a tightly controlled sequence during plant growth. This heterogeneous higher order structure of the cell wall impacts the cell wall's response to deconstruction and conversion.

Plant cell walls exhibit substantial heterogeneity in both content and distribution of the inorganic elements which also have implications for biomass conversion processes. This includes differences between content and distribution of inorganics in disparate plant taxa, differences between related species, within a single species as a function of its phenotype and environment, and even between tissues in a single plant.

Inorganic elements in plants are known to be responsible for diverse roles, including maintenance of ionic equilibrium in cells (e.g., K) and storage (e.g., Fe in ferritin), which, despite being localized in plastids, is water-extractable. A subset of the inorganic elements in plants is strongly associated with the cell wall. These elements are more resistant to aqueous extraction and include inorganic elements that may have structural roles, including, but not limited to, Ca and B ionic cross-links in pectic polysaccharides, calcium oxalate raphide crystals in some grasses, and Si in the cell walls of grasses which can comprise a significant fraction of the mass of a plant. Another class of role of cell-wall associated inorganic elements are metal co-factors in enzymes (e.g., Zn, Fe, Mn, Cu).

Redox-active metals, such as Cu, Mn, Fe, can exist in multiple oxidation states in vivo and are often involved in reactions involving electron transfer. Specifically, Fe in plants is associated with Fe-heme proteins and iron-sulfur (Fe—S) clusters in proteins, such as ferredoxins, which function as electron carriers in the photosynthetic electron transport chain. Cu in plants has diverse roles as a structural element in regulatory proteins, in photosynthetic electron transport, mitochondrial respiration, and Fe mobilization, among others. Metals may also be associated with metallothioneins (MTs) and phytochelatins (PCs), which are cysteine-rich polypeptides involved in either ameliorating the toxicity or controlling homeostasis of metals such as Fe, Ni, Cd, Zn, and Cu by coordination by thiols. In addition to its involvement with enzymes associated with the shikimic acid pathway and lignin biosynthesis, Mn is contained in a metallo-oxo cluster containing 4 Mn ions at differing oxidation states in the oxygen-evolving complex of photosystem II.

There are differences in the strength and nature of association of cell wall-associated metals. Specifically, Mn may be strongly associated with the cell wall and be present in “organic chelates” or “bound to lignin.” Alkali delignified hardwoods are known to have differences in the extractability of cell wall-associated Mn versus Fe using chelating compounds. Mg, which is a component of chlorophyll, is useful for photosynthesis and protein synthesis, although a portion of Mg may be bound to pectin or precipitated as salts in the vacuole, while the remainder is extractable with water.

During either oxidative delignification or biomass conversion processes where oxygen may be present, cell wall-associated transition metals can catalyze the formation of reactive oxygen species through Fenton chemistry. This catalytic activation of oxygen by transition metals has been shown to contribute to the oxidative scission of polysaccharides during alkaline-oxidative bleaching or delignification using H₂O₂ or O₂. As a result, precautions are taken during these processes through chelation and washing steps to remove metals and addition Mg salts and silicates to complex transition metals during these unit operations.

Therefore, a pretreatment process is typically used to alter and open up the cell wall matrix, to hydrolyze the hemicelluloses, and to alter the hemicelluloses. Pretreatment disrupts the non-easily digestible portion of lignocellulosic biomass, e.g., cellulose and lignin, thus improving its accessibility. After pretreatment, much of the biomass becomes easily digestible, while a portion remains non-easily digestible. Ultimately, the pretreatment process makes the cellulose more accessible (during a subsequent hydrolysis process, such as with lytic enzymes) for conversion of the lignocellulose polysaccharides (e.g., cellulose and hemicellulose) to monomeric sugars, which can be transformed to target products via catalytic conversion or microbial fermentation.

However, enzymatic hydrolysis of lignocellulose polysaccharides is usually hindered by the natural resistance of plant cell wall against deconstruction. To overcome this resistance, pretreatment processes of biomass feedstock have been developed and employed. Biomass pretreatment modifies cell wall structure and renders the biomass more digestible by enzymes.

A wide range of pretreatments are known, but few pretreatment methods have been identified as effective for biomass feedstocks, such as woody biomass, which are highly resistant to enzymatic hydrolysis. For example, enzymatic hydrolysis of hybrid poplar wood usually produces sugars at only 5 to 30% of the theoretical maximum yield. As noted above, such resistance involves the structural rigidity of the plant cell wall, the crystallinity of cellulose, and the presence of lignin, which remains as a residual material.

However, in the embodiments described herein, the alkaline pretreatments not only solubilize the lignin, it is expected that the lignin produced may closely resemble native lignin, such that less than 35% of the α-carbon of the solubilized lignin is oxidized from a hydroxyl to a carbonyl.

Lignin is known to be useful in a variety of applications including, but not limited to, carbon fiber composites, bio-oil, resins, adhesive binders and coating, plastics, paints, enriching soil organic carbon, fertilizer, rubbers and elastomers, paints, antimicrobial agents and slow nitrogen release fertilizer, and the like, and can be a substitute for polymers produced using crude oil.

One current source of lignin in the market is produced from sulfite (or sulfonate) based paper/pulp mills, a kraft pulping process, and the like. Most such mills currently burn the lignin to recover energy, in an attempt to reduce the environmental impact of discharge. Very few sulfite mills currently process the lignosulfonates from sulfite spent liquors. Additionally, the quality and quantity of lignin obtained via currently known methods are inadequate for most applications. As such, methods to fractionate and convert lignin into value-added products is needed.

Known methods for pretreating plant biomass are typically performed under elevated pressures and temperatures (above room temperature). Such methods include hot water and steam treatments, ammonia treatments and sulfite treatments.

Other pretreatment methods utilize an oxidant-based pretreatment, such as the alkaline oxidative pretreatment (AOP) process defined herein or a conventional alkaline hydrogen peroxide known by those skilled in the art. Yet other methods include catalytic processes. Catalytic approaches to plant cell wall deconstruction and conversion of insoluble biomass rely on homogeneous catalysts to allow the catalyst to diffuse through nano-scale pores within the cell walls to perform the desired reactions. Heterogeneous catalysis is known to be inefficient unless the cell walls are solubilized in expensive solvents such as ionic liquids. In one embodiment, homogeneous catalysts are used in many applications, such as homogeneous copper catalysts used for atom transfer radical polymerization where catalyst removal to prevent contamination of the product adds cost to the process.

Use of a single ligand copper complex as a catalyst in combination with an alkaline oxidative pretreatment (AOP) process with one oxidant is known. See, for example, Li et al., Rapid and Effective Oxidative Pretreatment of Woody Biomass at Mild Reaction Conditions and Low Oxidant Loadings Biotechnol Biofuels 6(1), 119 (2013), and Li, et al., Catalysis with Cu^{II}(bpy) Improves Alkaline Hydrogen Peroxide Pretreatment. Biotechnol Bioeng. 110(4):1078-1086 (2013), each of which is incorporated by reference in its entirety. However, the amount of oxidant required in such processes is high, such as at least 10% of the weight of the biomass to be treated. Additionally, in order to achieve suitable pretreatment results, the amount of metal utilized in a single-ligand copper complex is high (e.g., more than 50 μmol of metal complexes per gram of biomass to be pretreated). Use of such high levels of a metal can pose toxicity issues in subsequent processes (e.g., fermentation). Moreover, use of such high amounts of metals and oxidants can be cost prohibitive.

The various embodiments described herein provide for homogenous catalyst systems that are advantageously cost effective. Technoeconomic analysis (TEA) provided by the systems and methods described herein can significantly reduce the minimum fuel selling price (MFSP).

The various embodiments described herein provide a multi-ligand metal complex for use in an oxidative pretreatment process, such as an alkaline oxidative pretreatment (AOP) process, which not only allows for a reduction in the amount of metals used in the process, but also a reduction in the amount of oxidant. In one embodiment, the multi-ligand metal complex is used with a conventional alkaline hydrogen peroxide process. In one embodiment, the multi-ligand metal complex is a multi-ligand copper complex. In one embodiment, the copper complex is a copper(II) 2,2′-bipyridine complex (Cu(bpy)) modified to contain at least one additional metal-coordinating ligand, such as pyridine; 1,10-phenanthroline; ethylenediamene; histidine; and/or glycine.

The various embodiments described herein also provide a metal-ligand complex with a single ligand for use in an oxidative pretreatment process with two or more oxidants which not only allows for a reduction in the amount of metals used in the process, but also a reduction in the amount of expensive ligands and oxidants. In one embodiment, the metal-ligand complex is used with two co-oxidants in an alkaline oxidative process that includes hydrogen peroxide and oxygen (AHP/O). In one embodiment, the metal-ligand complex is a copper-ligand complex. In one embodiment, the copper complex is a copper 2,2′-bipyridine complex (Cu(bpy)). The Cu(bpy) catalyst may be further modified to contain at least one additional metal-coordinating ligand, such as pyridine; 1,10-phenanthroline; ethylenediamene; histidine; and/or glycine.

While not wishing to be bound by this proposed theory, both the single- and multi-ligand metal complexes are thought to function as suitable catalysts for lignocellulosic biomass (i.e., cause sufficient catalyst sorption into the biomass) due to the ability of the cationic metal, such as copper, to interact with (e.g., bond with) charged anionic groups, such as deprotonated phenolic hydroxyls in lignin, carboxylate groups in lignin, and/or uronic acids in pectins and hemicelluloses.

In one embodiment, the pH of the plant biomass being pretreated is adjusted to increase the number of deprotonated groups. In one embodiment, the pH of the pretreated biomass is, or the biomass is pH adjusted to achieve, a neutral pH during the pretreatment process. In one embodiment, the pH is adjusted to achieve an alkaline pH to deprotonate the phenolic groups in lignin and to increase lignin solubility. In one embodiment, the pH is adjusted to at least 11, such as at least 11.5, including any value in between. In some embodiments, elevation of the pH is achieved with bases such as ammonia and/or ammonia derivatives, such as amines, in which copper is stabilized in solution in the form of a complex ion. In one embodiment, the pH is adjusted via addition of a base, which can react with lignin and cause depolymerization and/or solubilization, i.e., helps the plant cell wall to become degraded and/or destroyed, thus reducing resistance to subsequent hydrolysis.

In one embodiment, oxidants useful in an oxidative pretreatment process include, but are not limited to, air, oxygen, hydrogen peroxide, ozone, persulfate, percarbonate, sodium peroxide and combinations thereof. In one embodiment, the metal-coordinating ligands include 2,2′-bipyridine and at least one of another metal-coordinating ligand, including, but not limited to nitrogen-donating ligands such as pyridine, 1,10-phenanthroline, and ethylenediamene, and ligands containing both a nitrogen donor and a carboxylate group such as the amino acids including histidine or glycine. In one embodiment, the catalytic metal element(s) (i.e., metal or metals) in the catalyst can include, but are not limited to, aluminum, zinc, nickel, magnesium, manganese, iron, copper cobalt and/or vanadium in various oxidation states. In various embodiments, the catalytic metal element is a metal ion(s). The metal ion(s) is redox active. The metal ions can be oxidized and/or reduced. In one embodiment, the elements include, but are not limited to, iron (e.g., Fe(II), Fe(III)), copper (e.g., Cu(I), Cu(II)), cobalt (e.g., Co(III), Co(VI)), and/or vanadium (e.g., V(II), V(III), V(IV), V(V)).

By substituting an amount of the 2,2′-bypyridine (bpy) with other, lower costs ligands, substantial savings can be achieved. In one embodiment, about 1 weight/weight (w/w) % up to about 99% or higher, such as 100% of bpy is substituted, such as about 10 to about 90%, such as about 20 to about 80%, such as about 35% to about 60%, including any range there between. In the various embodiments described herein, the multi-ligand metal complexes have low production costs. In one embodiment, substitution of bpy with other metal coordinating ligands provides a savings on the order of 10-fold or more, such as a savings of about 20 to 30 times the cost of using bpy alone.

An additional benefit relates to reduced microbial toxicity. Microbial toxicity is characterized by the final growth of yeast cells during yeast fermentation, and/or the growth rate of yeast during fermentation, and/or the length of the lag phase during fermentation. Such toxicity is caused by metal ions and other chemicals present in the processing stream, including the metals present in the multi-ligand catalyst and metal elements present in the plant biomass itself. Since the various embodiments allow for a reduced amount of metal as compared to conventional processes, the yeast used downstream is less adversely affected down to minimally adversely affected. As such, in one embodiment, the multi-ligand complexes have minimal microbial toxicity towards yeast fermentation (i.e., less than 50% reduction in final growth of yeast cells, as quantified with optical density).

In one embodiment, the final growth of yeast cells during yeast fermentation is decreased by 50% or less as the result of the presence of multi-ligand metal complexes, which indicates the low toxicity of the multi-ligand metal complexes. In one embodiment, the final growth of yeast cells is decreased by less than 40 to 50%, less than 30 to 50%, less than 20 to 50%, less than 10 to 50%, less than 5 to 50%, including any range or value there between. Additionally, the various embodiments provide for a pretreatment method which produces pretreated biomass which is easily digestible by commercial cellulase cocktails into fermentable sugars (glucose, xylose, etc.)

Use of the multi-ligand metal complexes described herein also reduces the amount of metal, such as copper, used in the process as compared to a single-ligand metal complex, such as a single ligand copper complex, by at least 50%, or at least 40%, or at least 30% or at least 20% or at least 10% or at least 5% or lower, including any range therein. Use of a reduced amount of metal not only reduces toxicity levels, but further reduces costs.

Use of the multi-ligand metal complex may reduce the amount of oxidant, such as hydrogen peroxide, used in the oxidative pretreatment by at least 90%, by at least 80%, by at least 70%, by at least 60%, by at least 50%, or at least 40%, or at least 30% or at least 20% or at least 10% or at least 5% or lower, including any range therein. Use of a reduced amount of oxidant further reduces costs.

In one embodiment, one or more oxidants are combined with the other reactants at a low weight percent (%) loading based on original biomass (w/w), i.e., loading of no more than 15%. In one embodiment, the oxidant loading is less than 10%, such as less than 5%. In one embodiment, the oxidant loading ranges from about 1% to about 15%, such as about 1% to about 10%, such as about 1% to 5% or less, including any range there between. Such loadings are lower than conventional loadings which can be as high as 200%. In one embodiment, at least 0.1% to about 50% w/w, such as greater than 31.5% oxidant is added, such as from about 31.51% to about 50% w/w. In one embodiment, at least 2.5% to about 50% w/w, such as greater than 31.5% hydrogen peroxide is added, such as from about 31% to about 50% w/w.

In one embodiment, use of the multi-ligand metal complex as a catalyst during an oxidative pretreatment may allow the pretreatment process to proceed significantly faster (e.g., at least two times as fast) as compared with an oxidative pretreatment performed using a conventional single-ligand metal complex as a catalyst.

In one embodiment, the oxidant is added at a “slow add” rate. In one embodiment, the “slow add” rate is equal to or less than a rate of consumption of the oxidant by the other reactants. Use of a slow rate, surprisingly, results in increased effectiveness of the pretreatment, as evidenced by increased downstream yields of various sugars.

In various embodiments, hydrolysis may optionally be followed by or integrated with either fermentation or sugar catalytic conversion of sugars to bioproducts, such as biofuels, biochemicals and biopolymers. Use of the metal complex as described herein provides improved downstream bioproduct yields, such as sugar yields, as compared to yields obtained with a single-ligand metal complexes, such as single-ligand copper complexes. In one embodiment, such yields may be improved by at least 5% or higher, such as at least 10%, at least 20%, at least 30%, at least 40% at least 50% or higher, up to two or three times higher, including any range there between.

### Dual Oxidant Process

In various embodiments, the oxidative pretreatment process with two or more oxidants described herein, e.g. O₂-enhanced alkaline hydrogen peroxide (AHP/O) pretreatment process, improves the economics of the oxidative lignocellulosic-to-biofuel technique. This process leads to a reduction in the loading of expensive chemical inputs in conjunction with an increase in the recovery of biomass polymers.

In various embodiments, the ligand metal complexes with one or more ligands with two or more oxidants can be used in the oxidative pretreatment process of plant biomass. The use of two or more oxidants in the oxidative pretreatment process will be described with reference to O₂-enhanced alkaline oxidative hydrogen peroxide (AHP/O) pretreatment process but it will be understood that other oxidant combinations may also be used and are within the scope of this description. In one embodiment, the oxidative pretreatment process uses hydrogen peroxide in combination with oxygen. In one embodiment, a portion of the hydrogen peroxide generally used in a single oxidant process can be replaced with the molecular oxygen. Reduction of the hydrogen peroxide in the oxidative pretreatment process can lead to a cost savings. In one embodiment, the hydrogen peroxide and the oxygen are present simultaneously in the oxidative pretreatment process.

In various embodiments, the loading of hydrogen peroxide in the AHP/O process can be less than about 10% w/w (H₂O₂/original biomass), or less than about 8% w/w, or less than about 5% w/w, or less than about 4% w/w, or less than about 3% w/w, or less than about 2% w/w, or less than about 1% w/w. In one embodiment, the loading of hydrogen peroxide in the AHP/O process is about 2% w/w.

In various embodiments, the amount of oxygen used to enhance the AHP/O process is greater than about 5 psi, or greater than about 10 psi, or greater than about 25 psi, or greater than about 40 psi, or greater than about 50 psi, or greater than about 75 psi. In various embodiments, the amount of oxygen used to enhance the AHP/O process is less than about 100 psi, or less than about 75 psi, or less than about 60 psi, or less than about 50 psi, or less than about 40 psi, or less than about 25 psi, or less than about 10 psi. In one embodiment, the amount of oxygen used to enhance the AHP/O process is about 50 psi.

In one embodiment, the oxidants used in the AHP/O process included hydrogen peroxide at about 2% w/w and oxygen at about 50 psi. Variations of this combination of concentrations may be used as described above and are within the scope of this description.

In various embodiments, the ligand metal complexes can include any of the metals described herein. In one embodiment, the ligand metal complexes can be copper ligand complexes. The ligand can be any ligand as described herein. In various embodiments, the ligand metal complexes can include a single ligand. In various embodiments, the ligand metal complexes can be multi-ligand complexes as described herein. In one embodiment, the ligand metal complex is a Cu(bpy) complex.

In various embodiments, the amount of ligand metal complex, e.g. Cu(bpy) complex, is less than about 2 mM, or less than about 1.5 mM, or less than about 1 mM, or less than about 0.8 mM, or less than about 0.5 mM. In one embodiment, the ligand metal complex is about 1 mM. The amount of the ligand metal complex is reduced by at least about 25%, or by at least about 50% or by at least about 75% compared to oxidative pretreatment process with one oxidant.

In one embodiment, prior to being subjected to a catalyzed pretreatment process the plant biomass can be first subjected to an alkaline pre-extraction step designed to facilitate oxidative pretreatment and biomass hydrolysis.

In various embodiments, the temperature at which the alkaline pre-extraction and/or at which the oxidative pretreatment is conducted can be increased to improve the economics of the pretreatment process. In one embodiment, the temperature at which the alkaline pre-extraction step is conducted and the temperature at which the oxidative pretreatment, e.g. AHP/O, step is conducted is increased compared to the processes with a single oxidant.

In various embodiments, the alkaline pre-extraction step, prior to the AHP/O process, is conducted at a temperature of about 30° C. or greater, or about 40° C. or greater, or about 50° C. or greater, or about 75° C. or greater, or about 90° C. or greater, or about 100° C. or greater, or about 110° C. or greater, or about 120° C. or greater, or about 130° C. or greater. In various embodiments, the alkaline pre-extraction step, prior to the AHP/O process, is conducted at a temperature of about 130° C. or less, or about 120° C. or less, or about 110° C. or less, or about 100° C. or less, or about 80° C. or less, or about 50° C. or less, or about 40° C. or less. In one embodiment, the alkaline pre-extraction step, prior to the AHP/O process, is conducted at a temperature of about 120° C.

In various embodiments, the AHP/O process is conducted at a temperature of about 30° C. or greater, or about 40° C. or greater, or about 50° C. or greater, or about 60° C. or greater, or about 70° C. or greater, or about 80° C. or greater, or about 90° C. or greater, or about 100° C. or greater. In various embodiments, the AHP/O process is conducted at a temperature of about 100° C. or less, or about 90° C. or less, or about 80° C. or less, or about 70° C. or less, or about 60° C. or less, or about 50° C. or less, or about 40° C. or less. In one embodiment, the AHP/O process is conducted at a temperature of about 80° C.

In one embodiment, the alkaline pre-extraction step is conducted at a temperature of about 120° C. and the AHP/O process is conducted at a temperature of about 80° C. Variation of these combinations of temperature may be used and are within the scope of this description.

Any suitable plant biomass can be used. In one embodiment, the plant biomass contains transition metals. Use of a plant biomass containing more than trace amounts of one or more transition metals results in further cost savings, as a reduced amount of catalyst is needed to affect the same or substantially the same results. Examples of plant biomass containing more than trace amounts of transition metals include, but are not limited to, hardwoods of the genus Populus (e.g., various types of poplar including hybrid poplar, hybrid aspen, western balsam poplar, and the like), birch (including silver birch and the like), maple (including sugar maple and the like), further including grasses (including, but not limited to, corn, switchgrass, Sorghum, Miscanthus) and gymnosperms, which are also referred to as conifers and softwoods (including, but not limited to, the genus of Pinus, such as Pinus resinosa, i.e., red pine).

In one embodiment, the plant biomass contains one or more transition metals that are redox-active, including, but not limited to, Fe, Mn, Cr, Co, Ni, Cu, Mo, Pd, Ru, Re, Pt, Pd, Os, Ir and combinations thereof.

In various embodiments, the biomass may be subjected to a cycle of hydrolysis (e.g., enzymatic, acid, etc.) using any conventional methods known in the art. In one embodiment, a reduced amount of enzymes is used, as compared to hydrolysis of conventionally catalyzed pretreated biomass.

In various embodiments, use of the metal complex as described herein with two oxidants, e.g. AHP/O, provides improved downstream bioproduct yields, such as sugar yields, as compared to yields obtained with ligand metal complexes with one oxidant, such as single-ligand copper complexes with only hydrogen peroxide as the oxidant. In one embodiment, such yields may be improved by at least about 5% or higher, such as at least about 10% or higher, at least about 20% or higher, at least about 30% or higher, at least about 40% or higher, at least about 50% or higher, up to two or three times higher, including any range there between.

In one embodiment, the use of AHP/O process can result in the using lower amounts of enzymes for enzymatic hydrolysis of the catalytically pretreated biomass than the catalytically treated biomass generated from oxidative pretreatment processes with one oxidant. In various embodiments, the amount of enzyme used for hydrolysis is less than about 50 mg protein/g glucan, or less than about 40 mg protein/g glucan, or less than about 30 mg protein/g glucan, or less than about 20 mg protein/g glucan, or less than about 15 mg protein/g glucan, or less than about 10 mg protein/g glucan. In various embodiments, the amount of enzyme used for hydrolysis is more than about 5 mg protein/g glucan, or more than about 10 mg protein/g glucan, or more than about 15 mg protein/g glucan, or more than about 20 mg protein/g glucan, or more than about 25 mg protein/g glucan. In one embodiment, the amount of enzyme used for hydrolysis is about 15 mg protein/g glucan.

In various embodiments, the overall monomeric sugar yields following enzymatic hydrolysis in the AHP/O process increased compared to processes using one oxidant. In various embodiments, the amount of xylose yield (based on the initial sugar composition) is more than about 70%, or more than about 80%, or more than about 90%, or more than about 95%, or about 100%. In various embodiments, the amount of xylose yield (based on the initial sugar composition) is about 100% or less, or about 95% or less, or about 90% or less, or about 85% or less.

In various embodiments, the amount of glucose yield (based on the initial sugar composition) is more than about 70%, or more than about 80%, or more than about 90%, or more than about 95%, or about 100%. In various embodiments, the amount of glucose yield (based on the initial sugar composition) is about 100% or less, or about 95% or less, or about 90% or less, or about 85% or less. In one embodiment, the amount of glucose yield is about 93% and the amount of xylose yield is about 100% (based on the initial sugar composition).

Use of the metal-ligand complexes described herein with two oxidants also reduces the amount of metal, such as copper, used in the process as compared to a process with a single-ligand metal complex with one oxidant, by at least 75%, or by at least 50%, or at least 40%, or at least 30% or at least 20% or at least 10% or at least 5% or lower, including any range therein. Use of a reduced amount of metal not only reduces toxicity levels, but further reduces costs.

Use of a ligand-metal complex with two oxidants, e.g. hydrogen peroxide and oxygen, may reduce the amount of the one of the more expensive oxidants, such as hydrogen peroxide, used in the oxidative pretreatment by at least 90%, or by at least 80%, or by at least 70%, or by at least 60%, or by at least 50%, or by at least 40%, or by at least 30% or by at least 20% or by at least 10% or by at least 5% or lower, including any range therein. Use of a reduced amount of oxidant further reduces costs.

In one embodiment, use of the ligand-metal complex as a catalyst during an oxidative pretreatment with two oxidants, e.g. AHP/O, may allow the pretreatment process to proceed significantly faster (e.g., at least two times as fast) as compared with an oxidative pretreatment performed using a conventional single-ligand metal complex as a catalyst with one oxidant.

In various embodiments, the use of two oxidants in the oxidative pretreatment process can result in favorable technoeconomic analysis (TEA) of the pretreatment of biomass to generate biofuels. In various embodiments, the improved TEA can result from conducting alkaline pre-extraction at a higher temperature, conducting the AHP/O process at a higher temperature, using two oxidants, hydrogen peroxide and oxygen, in conjunction with a single-ligand metal complex, e.g. Cu(bpy), reducing the amount of ligand used in the oxidative pretreatment process, reducing the load of the enzyme in the enzymatic hydrolysis with improved yield of monomeric sugars and/or a combination of these factors.

In one embodiment, improved TEA can result from conducting alkaline pre-extraction at about 120° C., conducting the AHP/O pretreatment process at about 80° C., using hydrogen peroxide and oxygen as oxidants in conjunction with Cu(bpy), and reducing the amount of the ligand to 1 mM, reducing the amount of hydrogen peroxide to 2% w/w (H₂O₂/original biomass) with oxygen at about 50 psi, reducing the load of the enzyme to 15 mg protein/g glucan in the enzymatic hydrolysis leading to an improved yield of monomeric sugars, e.g. about 93% glucose and about 100% xylose (based on the initial sugar composition).

TEA indicates that the improved conditions can reduce the minimum fuel selling price (MFSP) compared to a one oxidant process by more than about 20%, or by more than about 30%, or by more than about 40%, or by more than about 50%, or by more than about 60%, or by more than about 70%, or by more than about 80%, or by more than about 90%. In one embodiment, TEA indicates that the improved conditions can reduce the MFSP by about 30% to about 40% compared to a single oxidant process using H₂O₂.

In one embodiment, the process can further include recovery and reuse of the ligand metal complex, including recovery of the metal itself. Conventional technologies for metal removal (e.g., copper) from wastewater streams are based on ion exchange, precipitation/co-precipitation plus filtration, and membrane separation. Additionally, lignocellulose such as waste biomass or biomass fractions, such as lignin, have been proposed as biosorbant materials in the treatment of wastewater to remove heavy metals, including copper. Cationic metals can sorb to charged anionic groups such as deprotonated phenolic hydroxyls in lignin or carboxylate groups in lignin or uronic acids in pectins and hemicellulose and are known to be strongly affected by pH with more deprotonated groups at elevated pH.

In one embodiment, catalyst sorption to biomass is strongly pH-dependent with near-complete catalyst adsorption to biomass at alkaline pH and substantial desorption at neutral to acidic pH. In one embodiment, pH is adjusted to recover the multi-ligand metal complex. In one embodiment, untreated plant biomass is used as an adsorbent to both recover the catalyst and impregnate the catalyst into the untreated plant biomass (such as woody biomass, including, but not limited to, poplar, hybrid polar, and other trees).

In one embodiment, any conventional method is used to recover the catalyst from either the unhydrolyzed pretreated biomass (often referred to as “pretreatment liquor”) and/or the clarified (cell-free) stillage following fermentation and distillation. Such methods include, but are not limited to flocculation, precipitation, and filtration using a polyanionic flocculant (e.g., Betz-Dearborn MR2405 or Ondeo-Nalco 8702) which is commercially employed to remove heavy metals during wastewater treatment. Such methods can further include recovery by adsorption to a commercial ion exchange resin (e.g., Amberlyst™ 40Wet) which is used industrially to recover and recycle copper catalyst used in the production of adipic acid. In one embodiment, the catalyst is recovered and recycled.

In embodiments which include a sugar conversion step, recovery and reuse of the ligand metal complex provides the additional benefit of further reducing toxicity during subsequent sugar conversion steps. Recovering and recycling the ligand metal complex further helps to reduce costs.

In one embodiment, the process may produce monomeric aromatic compounds, such as, syringic acid, vanillin, syringaldehyde acid, vanillic acid. Such aromatic compounds are useful in a number of applications, such as food additives, polymer precursors, and various types of chemicals.

In one embodiment, the process may produce aliphatic acids, including, but not limited to formic acid, oxalic acid, acetic acid, lactic acid, succinic acid, azaleic acid. Such aromatic compounds are useful in a number of applications, such as food additives, polymer precursors, and fine chemicals.

The various embodiments will be further described by reference to the following examples, which are offered to further illustrate various embodiments. It should be understood, however, that many variations and modifications may be made while remaining within the scope of the various embodiments.

### Example 1

Screening with Metal Ligand Complexes

Screening of various metal ligand complexes (hereinafter “catalysts”) was performed via catalytic alkaline oxidative pretreatment (AOP) pretreatment of alkali-extracted switchgrass (AESG). To prepare AESG, 80 grams of untreated switchgrass (Panicum virgatum, cv. Cave-In-Rock, hammermilled to pass a 5-mm screen, and stored at a moisture content of approximately 5%) was soaked in an aqueous solution of 5 g/L NaOH at 80° C. for 2 hours (hrs). The weight-to-volume (w/v) solids loading of switchgrass during alkaline extraction was 10% (e.g., 10 g of biomass in 100 mL NaOH solution). After the alkaline extraction, the residual solid biomass was recovered via filtration through a 200-mesh steel cloth and washed with deionized water until the washing effluent had a neutral pH. The washed solids were recovered as AESG and air-dried for over 7 days.

Fe(III)-phthalocyanine (95%), Fe(III)-tetraphenylporphyrin (≥97%) and Fe(III)-tetrakis-pentafluorophenyl)-porphyrin (≥9%) catalysts were purchased from Sigma-Aldrich and used without further purification. [Al(III)(3,5²Bu₂-salophen)Cl] [3,5-^{t}Bu₂-salophen=N,N′-bis(3,5-di-tert-butylsalicylidene)-1,2-phenylenediamine], [Zn(II)(BPPA)Cl] Cl [BPPA=N,N-bis(6-pivaloylamido-2-pyridylmethyl)-N-(2-pyridylmethyl)amine], and [NEt₄] [Fe(III)(bpb)Cl₂] [H₂(bpb)=N,N-bis(2-pyridinecarboxamide)-1,2-benzene] catalysts were synthesized according to the methods described in (Gorski et al. Analytica Chimica Acta 2010, 665, 39; Harata et al. Journal of Coordination Chemistry 1998, 44, 311; Barnes et al. Journal of Chemical & Engineering Data 1978, 23, 349; Afshar et al. Inorganic Chemistry 2006, 45, 10347; Yang et al. Journal of Molecular Catalysis A: Chemical 2007, 266, 284), which are hereby incorporated by reference in their entireties, and verified by NMR (Agilent Direct Drive 2, 500 MHz) and/or UV-Vis spectral analysis (Hewlett Packard 8453 UV/Vis spectrophotometer). The Cu(bpy) catalyst was prepared in an aqueous solution by mixing cupric sulfate pentahydrate (Mallinckrodt, 99.8%) and 2,2′-bipyridine at a ligand:metal molar ratio (L/M) of 3:1.

To study the effect of catalytic AOP pretreatment on AESG, pretreatment was performed in a 100 g/L aqueous suspension of AESG in the presence of each catalyst. The loading of hydrogen peroxide (J. T. Baker, ACS reagent, as in an about 30 to about 32% w/v aqueous solution) on biomass is approximately 10% by weight. The concentration of each catalyst in the aqueous suspension of AESG is shown in Table 1. The reaction mixture contained 0.04 M Na₂HPO₄—NaOH buffer (prepared using disodium phosphate and sodium hydroxide) to maintain the pH at 11.5 during pretreatment. After 24 hrs of incubation at 30° C. without mixing, the pretreated switchgrass slurry was washed using deionized water to remove the alkaline phosphate buffer.

The enzymatic digestibility of AOP-pretreated switchgrass was estimated by the yield of glucose during the enzymatic hydrolysis of AOP pretreated AESG. The percentage yield was calculated on the basis of the glucan content in AESG prior to catalytic AOP pretreatment. The theoretical maximum hydrolysis yield was lower than 100% due to the loss of sugars in the washing step between catalytic AOP pretreatment and enzymatic hydrolysis.

For enzymatic hydrolysis, 500 μL of 1 M Na-citrate buffer (pH 5) and 40 μL of 10 mM tetracycline (Sigma-Aldrich) were added to AOP-AESG that was not dried after catalytic AOP pretreatment. Accellerase 1500 (42 FPU/mL; Danisco-Genencor, Palo Alto, Calif.) was added at 50 mg protein per gram of glucan in AESG. The total volume of the mixture (solid and liquid) was adjusted with deionized water to reach a 5% (w/v) solids concentration, and the samples were incubated at 50° C. during hydrolysis.

The efficacy of catalysts on AOP pretreatment of AESG is shown in Table 1. AOP pretreatment of AESG at pH 11.5 was not improved via addition of metal catalysts, except for the case of Cu (bpy) catalyst, which gave a very moderate improvement in the enzymatic digestibility of AOP pretreated AESG. See Table 1 below.

TABLE 1
Efficacy of ligand-metal catalyst addition
to a 24 hour (hr) AOP Pretreatment of AESG

| | | | Glucose yield of enzymatic | |
|---|---|---|---|---|
| | Catalyst | | hydrolysis (% of theoretical | |
| | concentration | | maximum) | |
| Catalyst | (mM) | pH | 24 hrs | 72 hrs |
| No catalyst | — | 3 | 30.06 ± 1.96 | 36.12 ± 2.33 |
| | | 9 | 32.37 ± 1.2 | 37.97 ± 0.57 |
| | | 10 | 31.25 ± 0.16 | 38.77 ± 0.28 |
| | | 11 | 37.74 ± 0.33 | 42.70 ± 0.59 |
| | | 13 | 36.82 ± 1.23 | N.D. |
| Fe(III)- | 0.05 | 3 | 30.91 ± 0.24 | 36.80 ± 2.84 |
| phthalocyanine | | 11 | 34.21 ± 0.23 | 38.75 ± 1.52 |
| Fe(III)- | 0.05 | 3 | 31.55 ± 1.6 | 42.13 ± 1.89 |
| tetraphenylporphy- | | 11 | 36.98 ± 1.3 | 42.14 ± 0.63 |
| rin | | | | |
| Fe(III)-tetrakis- | 0.05 | 3 | 29.18 ± 0.33 | 35.38 ± 0.43 |
| pentafluorophenyl)- | | 11 | 35.37 ± 0.45 | 40.25 ± 0.19 |
| porphyrin | | | | |
| [NEt₄][Fe(III)(bpb) | 2 | 9 | 27.07 ± 0.6 | 34.27 ± 0.21 |
| Cl₂] | | 10 | 25.54 ± 0.21 | 31.97 ± 0.13 |
| | | 11 | 27.44 ± 1.54 | 31.54 ± 0.23 |
| [Al(III)(3,5-^{t}Bu₂- | 2 | 10 | 28.90 ± 0.17 | N.D. |
| salophen)Cl] | | 11 | 29.80 ± 0.25 | N.D. |
| | | 13 | 34.19 ± 0.41 | N.D. |
| [Zn^{II}(BPPA)Cl]Cl | 2 | 10 | 26.43 ± 0.31 | N.D. |
| | | 11 | 27.33 ± 0.62 | N.D. |
| | | 13 | 33.36 ± 0.3 | N.D. |
| Cu(bpy), L/M = 3:1 | 5 | 10 | 38.67 ± 0.3 | 42.98 ± 0.88 |
| | | 11 | 41.65 ± 0.14 | 44.97 ± 0.42 |
| | | 13 | 41.56 ± 0.06 | 43.95 ± 0.17 |

(N.D.: not determined)

### Example 2

Catalyst Screening with Multi-Ligand Metal Complexes

Metal complexes (hereinafter “metal catalysts”) containing multiple or single metal coordinating ligands were tested. The metal catalysts tested included copper catalysts having a second metal coordinating ligand substituted for at least a portion of the 2,2′-bipyridine (bpy). These catalysts were evaluated for their performance in further enhancing the efficacy of AOP pretreatment of hybrid poplar. Ligands involved in the screening included histidine, glycine, di(2-picolyl)amine (dipica, 97%), 1,4,7-triazacyclononane (TACN, 95%), 2,2′;6′,2″-terpyridine (Terpy, 98%), 1,10-phenanthroline (phen, ≥99%), and tris(2-pyridylmethyl)amine (TPA, 98%, obtained from Sigma-Aldrich and Acros Organics/0). An iron 2,2′-bipyridine complex was prepared by mixing ferrous sulfate heptahydrate with 2,2′-bipyridine in an aqueous solution, and this iron complex was also tested.

The hybrid poplar (Populus nigra var. charkoviensis x caudina cv. NE-19) used in this testing was grown at the University of Wisconsin Arlington Agricultural Research Station. The hybrid poplar was harvested in 2011 and debarked using methods known in the art at the University of Wisconsin, Madison to produce heartwood and sap wood (different parts of the wood stem which can be differentiated via chemical testing).

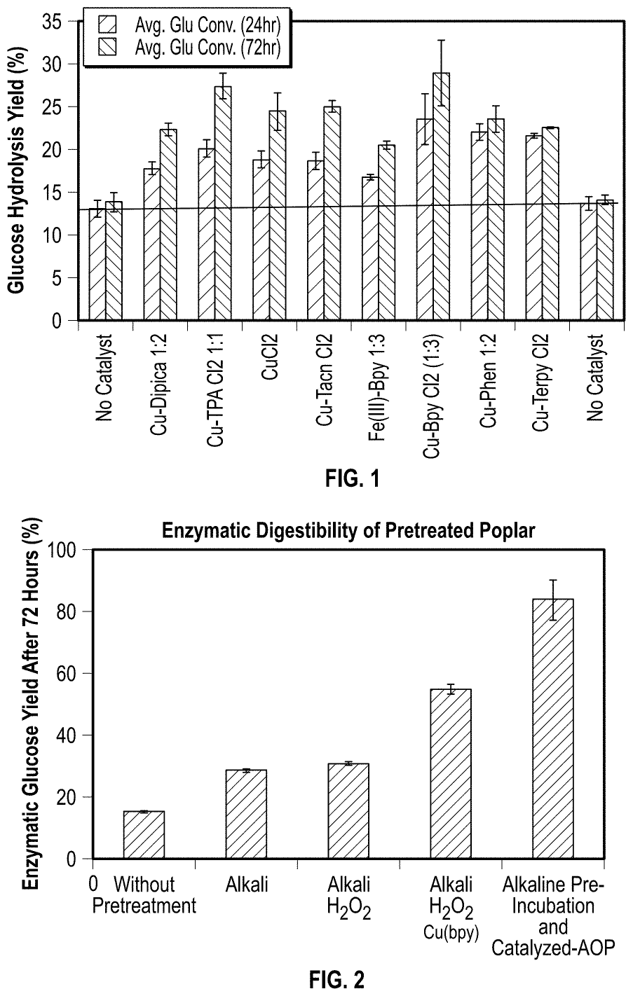

The enzymatic digestibility of hybrid poplar after pretreatment with alkaline oxidative pretreatment (AOP) and metal complexes involving above mentioned ligands was used to evaluate the performance of various multi-ligand metal complexes (See FIG. 1). As these results show, various types of multi-ligand metal complexes, as well as a single-ligand iron complex are capable of enhancing oxidative pretreatment of hybrid poplar. However, Cu(bpy) complexes demonstrated superior performance among all candidates, and were selected for further testing.

### Example 3

### Alkaline Extraction, Catalytic Pretreatment and Enzymatic Hydrolysis of Hybrid Poplar

About 0.5 g of hybrid poplar, i.e., poplar (from the same source as described in Example 2) was extracted in a temperature controlled rotary shaker (New Brunswick Scientific Classic Series C24KC, 210 rpm) at 30° C. in a 5 mL aqueous solution containing 270 mM NaOH (J. T. Baker, ACS reagent, 98.8%) for approximately 1 hr to produce extracted wet poplar. The extracted wet poplar was washed with 5 mL deionized water and centrifuged (Thermo Scientific Sorvall ST-16R) at 1000 rpm for 1 min to separate the solid and liquid phases. (The alkaline liquid from the washing was used in later examples for the extraction step).

The separated solid phase of the extracted wet poplar was then subjected to a catalytic AOP pretreatment in a 5 mL aqueous solution containing 10.8 g/L NaOH, 10 g/L hydrogen peroxide and a conventional single-ligand copper diimine catalyst at varying concentrations of copper (0.5, 1, 2, 3, 4, and 5 mM) and varying concentrations of 2,2′-bipyridine (1, 2, 4, 6, 8, 10, 15 and 20 mM) to produce incubated catalyzed pretreated (solid phase) poplar (hereinafter “solid poplar slurry”). The results presented herein are for concentrations of 2 mM of copper and 4 mM of 2,2′-bipyridine.

Enzymatic hydrolysis was performed on the solid poplar slurry at 50° C. in the same rotary shaker used for the extraction step. In preparation for hydrolysis, 20 μL of 72% (w/w) H₂SO₄ and 500 μL of 1 M sodium citrate buffer (pH 5) (prepared from sodium citrate and citric acid) were added to the solid poplar slurry to adjust the pH to 5 (optimum pH for the enzymes being used herein). Thereafter, 40 μL of 10 mM tetracycline (Sigma-Aldrich) stock solution was added to the solid poplar slurry to inhibit microbial growth. This step was followed by addition of a Cellic CTec2 and HTec2 (Novozymes A/S, Bagsværd, DK) enzyme cocktail at a loading of 30 mg protein/g glucan in the biomass prior to extraction and catalytic pretreatment, unless otherwise noted. The enzymatic reaction (50° C., 210 rpm, 72 hrs) was carried out at 5% solids concentration by adjusting the volume of the poplar slurry to 10 mL via the addition of deionized water.

Following enzymatic hydrolysis, the resulting solid and liquid phases were separated by centrifugation in same manner as in Example 2, to produce separated hydrolyzed solid and liquid phases. The amount of glucose and xylose released into the aqueous phase was quantified by High Performance Liquid Chromatography (HPLC) (Agilent 1260 Series equipped with an Aminex® HPX-87H column operating at 65° C., a mobile phase of 0.05 M H₂SO₄, and a flow rate of 0.6 mL per minute, and detection by refractive index). The yield of glucose and xylose released was defined as the amount of solubilized monosaccharide divided by the total sugar content of the poplar biomass prior to incubation and catalytic pretreatment as determined by chemical composition analysis.

After the enzymatic hydrolysis of the solid poplar slurry, over 80% of the polysaccharides (by weight) in the poplar biomass were converted to fermentable sugars. This sugar yield was significantly higher than the sugar yield produced using untreated raw hybrid poplar. (See FIG. 2).

### Example 4

### Recovery of Copper Catalyst, and Catalyst Impregnation During Alkaline Extraction

About 40 g hybrid poplar (same source as Example 2) was catalytically pretreated with 400 mL of aqueous solution containing 2 mM copper sulfate, 4 mM 2,2′-bipyridine (bpy), 270 mM NaOH and 10% (w/w) hydrogen peroxide loading on original biomass at 30° C. for 24 hrs to produce catalytically pretreated poplar slurry (hereinafter “poplar slurry”). Following the pretreatment, a copper removal step was performed to remove part or all of the copper present in the poplar slurry for use again in the process.

The copper removal step used in this testing was a desorption step in which the pH of the poplar slurry was adjusted to 5 with 1.6 mL of 72% sulfuric acid (prepared via dilution of 98% sulfuric acid, J. T. Baker) to produce a pH-adjusted poplar slurry. The pH-adjusted poplar slurry was then incubated for approximately 1 hr in the same rotary shaker as Example 2 at 30° C., with 120 rpm shaking speed to produce pH-adjusted poplar slurry. After incubation, the residual solid poplar was separated from the aqueous phase via centrifugation in the same manner as described in Example 3. The separated solids were then converted to sugars via enzymatic hydrolysis following the procedures described in Example 3.

The separated aqueous phase or pretreatment liquor (PTL) produced following centrifugation containing the copper catalyst (containing about 0.9 mM of coordinated copper complexes) was collected and used again in another catalytic AOP pretreatment. In this testing, about 0.5 g of hybrid poplar (same source as in Example 2) was incubated in the rotary shaker (120 rpm) with a mixture of 4.73 mL of the previously collected aqueous phase and 270 μL of 5 M NaOH (J. T. Baker, ACS reagent, 98.8%) at 30° C. for about 1 hr to produce a catalyst-impregnated wet poplar slurry. The catalyst-impregnated wet poplar slurry was thereafter washed with 5 mL deionized water and centrifuged in the same manner as Example 2 to separate the solid and liquid phases.

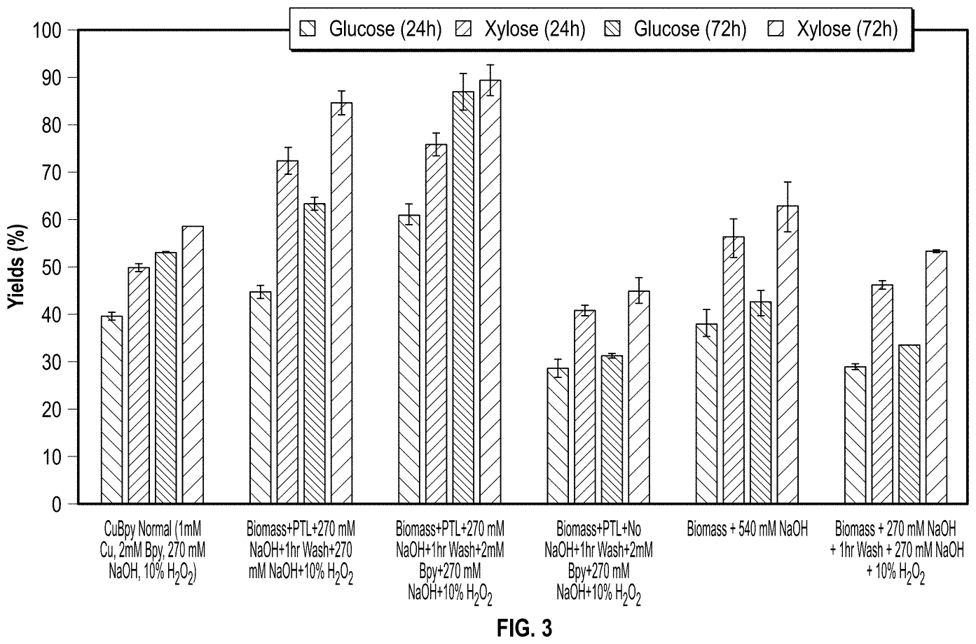

After another centrifugal solid-liquid separation (performed in same manner as described in Example 3), the separated solid phase of the incubated wet poplar biomass was catalytically pretreated with 5 mL aqueous solution containing 2 mM 2,2′-bipyridine, 270 mM NaOH and hydrogen peroxide (sources of chemicals used are as described in Example 3) at 30° C. for approximately 24 hrs. After the catalytic pretreatment, the biomass slurry was hydrolyzed by enzymes to obtain fermentable sugars at high yields (≥80% of the theoretical maximum). See FIG. 3.

### Example 5

### Production of Aromatic Chemicals Via Catalytic Oxidative Pretreatment

About 0.5 g hybrid poplar (same source as Example 2) was catalytically pretreated with 5 mL of an aqueous solution containing 2 mM copper sulfate, 4 mM 2,2′-bipyridine, 270 mM NaOH and 10% (w/w) hydrogen peroxide loading on biomass at 30° C. for approximately 24 hrs to produce a catalytically pretreated poplar slurry (sources of chemicals used are as described in Example 3). The solid and liquid phases were separated as described in the above examples.

For LC-MS analysis of the monomeric aromatic compound content, 10 μL of undiluted pretreatment liquor sample were injected into a XEVO G2SQTOF mass spectrometer in combination with a Waters Acquity® Ultra Performance Liquid Chromatograph (UPLC) system, which was equipped with an ESI interface capable of operating in both positive- and negative-ion modes. Chromatographic separation was carried out on a Thermo BetaBasic 100×2.1 mm C18 column (Thermo Fisher Scientific, Waltham, Mass., USA) maintained at 40° C. The binary solvent gradient comprised 0.1% formic acid in water (solvent A) and 100% methanol (solvent B) in the following gradient: 95% solvent A for the first 3 min, 50% solvent A over the next 1 min, 30% solvent A over next 2 min, and 5% solvent A over the final 2 min. The column was then returned to 95% solvent A, and equilibrated for 2 min prior to the next injection. A solvent delay of 2 min was used to prevent saturation of the detector with the sample solvent. The negative-ion mode mass spectrometry conditions were constant during all experiments with a voltage of −2.25 kV and a desolvation temperature of 350° C.

MassLynx™ software (Waters) version 4.1 was used for system control and data acquisition. The raw data was processed using the TargetLynx™ application. Pure standards for vanillin, vanillic acid, acetovanillone, syringaldehyde, syringic acid, acetosyringone, and p-hydroxybenzoate (Sigma-Aldrich, St. Louis, Mo., USA) were used to validate peak compound identification and for quantitation.

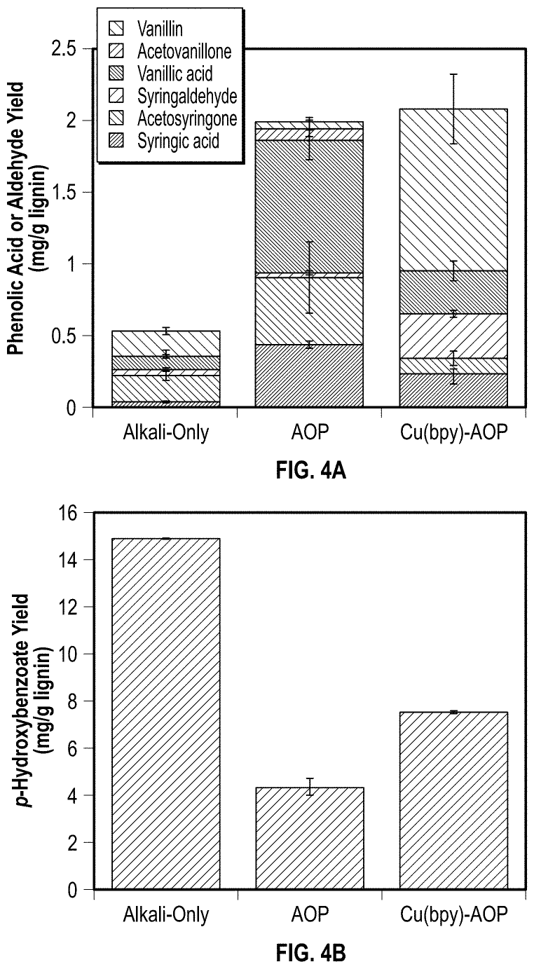

For quantitation of p-hydroxybenzoate, samples were prepared following the same procedure used for the LC-MS analysis, with the samples instead analyzed via high-performance liquid chromatography (HPLC) (Agilent 1260 LC equipped with an Agilent Poroshell 120 EC-C18 column (4.6×50 mm) and a diode array detector (DAD). Integration of the p-hydroxybenzoate peak at 280 nm and comparison to a standard curve was used for quantitation. A binary isocratic solvent system was utilized consisting of 80:20 solvent C to solvent D, where solvent C is acetonitrile with 0.1% water, and solvent D is acetonitrile with 0.1% trifluoroacetic acid. The results are shown in FIGS. 4A and 4B.

### Example 6

### Catalytic Pretreatment of Hybrid Poplar Biomass Using Copper (II) 2,2′-Bipyridine Ethylenediamine (Cu(Bpy)En)

Hybrid poplar (0.5 g) (same source as Example 2) was catalytically pretreated using 1 mM of a stock solution of multi-ligand copper complex, namely copper (II) 2,2′-bipyridine ethylenediamine (Cu(bpy)en) containing 40 mM copper sulphate pentahydrate, 40 mM bipyridine and 40 mM ethylenediamine in deionized water, together with 270 mM NaOH and 10% H₂O₂ loading (w/w) on biomass in a total 5 mL volume made with deionized water. Other components of the pretreatment catalyst include tetraacetylethylenediamine (TAED), ethylenediaminetetraacetic acid (EDTA), manganese sulfate, and magnesium sulfate. The catalytic pretreatment was allowed to proceed for approximately 24 hrs at 30° C.

Thereafter, the resulting poplar slurry was pH adjusted to 5 and hydrolyzed according to the process described in Example 3. Following enzymatic hydrolysis, the solid and liquid phases were separated by centrifugation as described in Example 3.

Among all catalyst systems tested, Cu(bpy)en yielded higher glucose yields compared to other catalyst systems tested. Also, by utilizing ½ the concentration of copper and ¼ the concentration of (bpy), as compared with a conventional Cu(bpy) catalyst, the overall cost is reduced. See Table 2 below.

TABLE 2
Catalyst and resulting glucose conversions

| Catalyst | Glucose conversions (%) |
|---|---|
| Cu(bpy)₂ | 55.76 |
| Cu(bpy)TAED | 55.01 |
| Cu(bpy)en (1 mM) | 55.00 |
| Cu(bpy)en (2 mM) | 48.15 |
| Cu(bpy)MnSO₄ | 46.78 |
| CuSO₄ | 40.62 |
| Mn(TAED)₂ | 38.65 |
| Cu(en)₂ | 34.22 |
| Cu(TAED)₂ | 30.72 |
| MgSO₄ | 25.17 |
| Mg(bpy) | 25.09 |
| Mn(en)₂ | 24.08 |
| Mn(EDTA)₂ | 22.28 |
| Mn(bpy)₂ | 21.21 |
| Control | 11.34 |

### Example 7

### Copper Recovery, Recycling and Extractives Removal Following Catalytic AOP Pretreatment Catalytic AOP Pretreatment

In one set of tests, 40 g hybrid poplar was catalytically pretreated at 10% biomass solids content (w/vol or “solids loading”) with a reaction mixture containing 2 mM copper sulfate pentahydrate, 4 mM 2,2′-bipyridine, 270 mM NaOH and 10% (w/w) H₂O₂ loading on biomass at 30° C., pH 11.5 for 24 hrs to produce pretreated poplar. Desorption of adsorbed copper from pre-treated poplar was carried out by reducing the pH of the pretreated poplar biomass to a pH of 5 by adding 1.6 mL of 72% H₂SO₄. The reaction mixture was catalytically pretreated in the same rotary shaker described above at 30° C., 120 rpm for 1 hr. After catalytic pretreatment, the resulting pretreated poplar slurry was centrifuged to separate the pretreated poplar from the aqueous pretreatment liquor (PTL), i.e., the aqueous phase collected following catalytic pretreatment. The PTL was collected to use for subsequent pretreatment step. One (1) mL of sample was analyzed with inductively coupled plasma mass spectrometry (ICP-MS; Thermoscientific iCAP 6500 ICP) to determine the concentration of copper in the PTL, which was found to be about 0.9 mM.

### Pretreatment Using the PTL

PTL (4.33 mL) was mixed with 0.5 g hybrid poplar (10% solids loading) with copper catalyst (Copper:bipyridine) of varying ratios. NaOH (270 mM) and H₂O₂ (10% loading on biomass) were added to the reaction mixture and the pretreatment lasts for 24 hrs at 30° C. After incubation, the biomass slurry was enzymatically hydrolyzed to yield monomeric sugars.

Due to the presence of 50% of originally added copper in the PTL, the slurry was loaded with different copper (≤1 mM) and 2,2′-bipyridine (≤2 mM) concentrations to match the concentrations with conventional AHP pretreatment (Copper 2 mM and bipyridine 4 mM). Results demonstrated that there were 50% less yields as compared to an AOP pretreatment in which a single ligand was used. The same yields were also obtained for different extra loadings of Cu and bpy as obtained with just PTL (FIGS. 4A and 4B).

These results suggest that inhibition is occurring, which could be due to the presence of extra free copper during enzymatic hydrolysis.

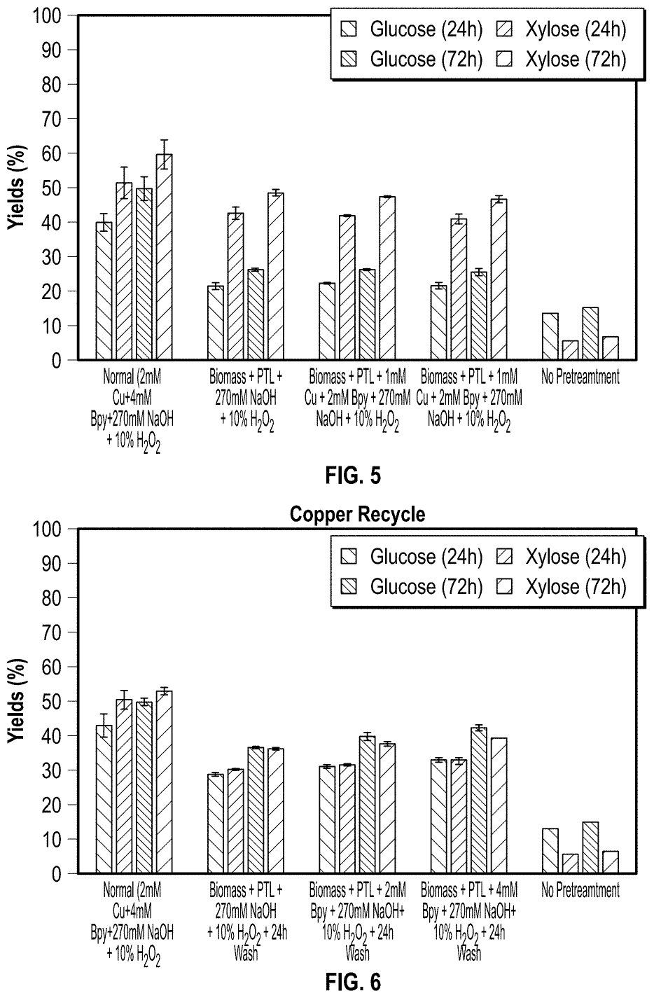

This testing shows that using the catalyst in PTL for oxidative pretreatment improves the enzymatic digestibility of pretreated biomass by about 5% (FIG. 5). As demonstrated in other testing described in Example 8, supplementing more catalyst in addition to PTL does not improve the efficacy of the catalytic oxidative pretreatment.

### Example 8

Other approaches beyond the approach described in the above examples were also used to pretreat the poplar biomass utilizing PTL, with the preferred approach determined on the basis of higher sugar yields after enzymatic hydrolysis.

In a second approach, PTL was added to the 0.5 g hybrid poplar biomass (10% solids loading on biomass by weight) with or without 2 mM bipyridine. NaOH (270 mM) and H₂O₂ (10% loading on biomass by weight) were added to the reaction mixture to carry out a catalytic pretreatment for 24 hrs at 30° C. to produce a pretreated poplar slurry which was washed using 5 mL distilled water. The washed pretreated poplar slurry was centrifuged in same manner as described in earlier Examples to separate the solid and liquid phases. The solid phase was enzymatically hydrolyzed to obtain the sugar yields.

In a third approach, 0.5 g of hybrid poplar (10% solids loading on biomass by weight) was catalytically pretreated with 4.73 mL of PTL and 270 mM NaOH at 30° C., 120 rpm for 1 hr to produce pretreated poplar slurry. The pretreated poplar slurry was washed with 5 mL of distilled water and centrifuged to separate the copper-containing aqueous phase from the solid phase. The solid phase (containing an amount of adsorbed copper) was subject to an additional incubation with 2 mM bipyridine, 270 mM NaOH and 10% H₂O₂ at 30° C., pH-11.5 for 24 hrs. Thereafter, the incubated pretreated poplar slurry was enzymatically hydrolyzed to yield monomeric sugars.

No additional copper was added in the pretreatment utilizing these different approaches. Control experiments were run to ensure the recovery and adsorption of copper into the new biomass.

### Extractives Removal and Alkali Recovery Experiment

On the basis of results obtained from above defined approaches, a two-step pretreatment process was designed. As such, 0.5 g of hybrid poplar biomass (10% w/v insoluble solids concentration) was pre-incubated with 270 mM NaOH at 30° C. for 1 hr. After incubation, contents of the reaction were washed with 5 mL water and centrifuged to separate solid and liquid phases. Solid phase, i.e., alkali pre-extracted biomass was subjected to catalytic AOP pretreatment, while the liquid phase was recovered in the same manner as described in previous examples. The pH of all the pretreatment reactions were carefully monitored and documented.

### Enzymatic Hydrolysis

Enzymatic hydrolysis was performed at 50° C. in a temperature controlled incubator shaker at 210 rpm for 72 hrs. After pretreatment, 20 μL of 72% (w/w) H₂SO₄ and 500 μL of 1 M sodium citrate buffer (pH 5.0) were added to the pretreated slurry to adjust the pH to 5 (optimum pH for enzymes). Next, 40 μL of 10 mM tetracycline(Sigma-Aldrich) stock solution was added to inhibit microbial growth, followed by addition of the enzyme cocktail consisting of Cellic CTec2 and HTec2 (Novozymes A/S, Bagsværd, DK) at a loading of 30 mg protein/g glucan each on the untreated biomass unless otherwise noted.

The enzymatic reaction was carried out at 5% solids loading by adjusting the volume to 10 mL by the addition of deionized water. Following enzymatic hydrolysis, the solid and liquid phases were separated by centrifugation, and the amount of glucose and xylose released into the aqueous phase was quantified by HPLC (Agilent 1100 Series equipped with an Aminex® HPX-87H column operating at 65° C., a mobile phase of 5.0 mM H₂SO₄, a flow rate of 0.6 mL/min, and detection by refractive index). The yield of glucose and xylose released was defined as the amount of solubilized monosaccharide divided by the total sugar content of the biomass prior to pretreatment as determined by chemical composition analysis.

ICP-MS metal analysis results showed that PTL contained 0.9 mM copper which was approximately 50% of copper concentration (2 mM) originally added during the pretreatment.

### Results

Results of the second approach demonstrated that there was an increase in the glucose yields compared to the yields obtained from the first approach described in the other examples. Addition of extra 2,2′-bipyridine further increased the yields. There was 6 to 8% increase in the glucose yield whereas lower xylose yield was obtained. Low xylose yields could be explained as due to washing step after pretreatment, hemicellulose was also removed from the reaction mixture.

No extra copper was added in these experiments. Yields obtained were purely from recycled copper.

These yields matched the yields achieved with conventional AOP pretreatment, i.e., one-step catalytic AOP with no extraction, incubation or impregnation beforehand. Increased yields were obtained with addition of bipyridine in the form of a bipyridine-copper complex.

The third approach (described above) produced the highest sugar yields as compared to the other approaches. The glucose yields obtained from this approach were approximately 28% higher compared to conventional catalytic AOP. Addition of only PTL to biomass without any addition of bipyridine yielded 7% higher glucose yields compared to conventional catalytic AOP.

No extra copper was added in these experiments. Yields obtained were purely from recycled copper.

These results show that extractives from PTL can inhibit the performance of catalytic AOP by affecting the pH of the reaction and/or by interfering with useful reactions. It is also possible that enzymatic hydrolysis was inhibited due to the presence of extractives.

Overall results demonstrated the ability to recycle the copper and increase the glucose yields by about 82% to about 85% and xylose yields by about 55% to about 58%.

### Modification of Catalytic AOP

On the basis of results obtained from copper recycling and extractives removal, a two-step process was developed, with the first step removing extractives and alkali-extractable lignin and xylan with NaOH and the second step consisting of catalytic AOP.

Sugar yields obtained from modified catalytic AOP were about 28 to about 30% higher than sugar yields obtained using conventional single-ligand catalytic AOP. Different control experiments were also carried out to ensure that the yields obtained are due to extractives removal, see FIG. 6.

See also improved sugar yields with pre-extraction step shown in FIG. 7.

### Example 9

### Recycling of Base for Alkaline Pre-Extraction

Alkaline pre-extraction was performed following the procedure described in Example 3. Hybrid poplar (from the same source as in previous examples) was extracted with 135 mM or 270 mM aqueous solution of NaOH, and the extracted biomass was then washed with deionized water. The liquid from washing was recovered. The washed solids were pretreated with 10.8 g/L NaOH, 10 g/L H₂O₂, 2 mM CuSO₄ and 4 mM 2,2′-bipyridine. The pretreated solids were enzymatically hydrolyzed following the procedure described in Example 3. Over 80% of the glucose was recovered after enzymatic hydrolysis (FIG. 7).

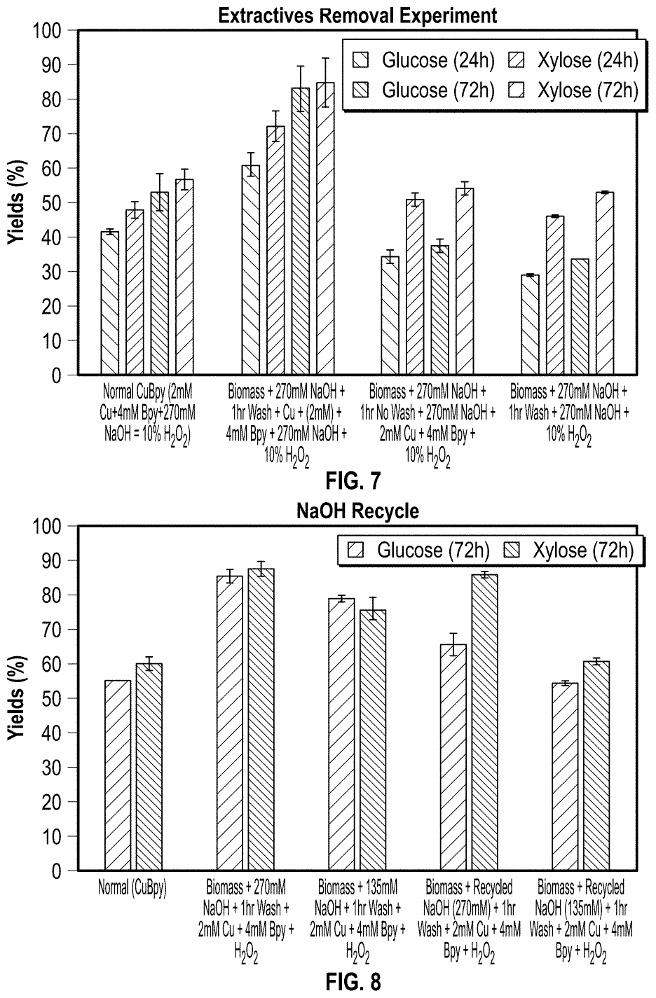

The liquid from washing (“Recycled NaOH”) was used for alkaline pre-extraction of hybrid poplar, following the procedure described in Example 3. After 1 hr of extraction at 30° C., the mixture was washed with deionized water, and the remaining solids was pretreated with 10.8 g/L NaOH, 10 g/L H₂O₂, 2 mM CuSO₄ and 4 mM 2,2′-bipyridine. The pretreated solids were enzymatically hydrolyzed following the procedure described in Example 3. More than 60% of the glucose was recovered after enzymatic hydrolysis (FIG. 8).

Compared to conventional single-ligand catalytic AOP, consumption of alkali was doubled in this modified catalytic AOP. To be more cost effective, experiments were performed to recycle the alkali from pre-extraction step or use less alkali in the respective step.

### Results

Sugar yields obtained using less alkali, i.e., 135 mM NaOH, were comparable to the yields obtained from the 270 mM NaOH pre-extraction step. The NaOH was also partially recycled. Yields obtained from the recycled 270 mM NaOH reaction were about 66% glucose and 85% xylose. These yields are higher than yields obtained with the conventional single-ligand catalytic AOP process (FIG. 8).

### Example 10

### Biomass, Pretreatment, and Hydrolysis

Hybrid poplar (Populus nigra var. charkoviensis x caudina cv. NE-19) was grown at the University of Wisconsin Arlington Agricultural Research Station. Prior to pretreatment, a mixture of heartwood and sapwood of the 18-year-old poplar was hammer-milled to pass a 5 mm screen. See, for example, Li Z, Chen C H, Liu T, Mathrubootham V, Hegg E L, Hodge D B: Catalysis with Cu^{II}(bpy) improves alkaline hydrogen peroxide pretreatment. Biotechnol Bioeng 2013, 110:1078-1086.37 and Li Z, Chen C, Hegg E, Hodge D: Rapid and effective oxidative pretreatment of woody biomass at mild reaction conditions and low oxidant loadings. Biotechnol Biofuel 2013, 6:119, for additional details as to compositional analysis and pretreatment specifics. For AOP-only pretreatment, hybrid poplar (0.5 g) was pretreated in 5 mL aqueous aliquots of 10 g/L H₂O₂ (10% w/w loading on biomass) and 10.8 g/L NaOH (final pH of approximately 11.7) at 30° C. for 1 hr unless otherwise noted. During the pretreatment, the samples were agitated at 180 rpm in an orbital shaker. For Cu-catalyzed AOP pretreatments, 5 mM CuSO₄ and 25 mM 2,2′-bipyridine were included in the 5 mL aliquot during pretreatment. For alkali-only (AOP-only) pretreatment, 0.5 g of hybrid poplar was pretreated in 5 mL aqueous aliquots of 10.8 g/L NaOH.

### TEM Imaging and Elemental Profiling of Pretreated Cell Walls

Structural modification of hybrid poplar cell wall by pretreatment was studied using transmission electron microscope (TEM) combined with energy-dispersive X-ray spectroscopy (EDS) and electron energy-loss spectroscopy (EELS). The conditions used for pretreatment were identical to those used to prepare size exclusion chromatography (SEC) samples. Cell wall samples of untreated hybrid poplar and hybrid poplar treated with AOP and Cu-catalyzed AOP for 24 hrs were air dried and fixed (i.e., embedded) in a resin comprising 0.1 M pH 7 phosphate buffer containing 2.5% (w/w) glutaraldehyde and 2.5% (w/w) paraformaldehyde. The fixed cell wall samples were embedded in Spun epoxy resin (Poly/Bed 812, Polysciences) and sectioned to 100 nm thickness using a PowerTome XL ultramicrotome (Boeckeler Instruments, Tucson, Ariz., USA). Thin sections were placed on 150 mesh gold grids with Formvar/carbon support film (Electron Microscopy Sciences, PA) and stained in 1% aqueous solution of KMnO₄ for 1 min.

Samples were then rinsed with deionized water to remove excess stain. Bright field TEM micrographs and EELS spectra were acquired under a JEOL 2200FS 200 kV field emission TEM (Peabody, Mass., USA) fitted with a Gatan (Warrendale, Pa., USA) digital multi-scan camera. EDS spectra were acquired using an Oxford INCA system (Oxford Instruments, Abington, UK) coupled with the TEM.

### Analysis or Pretreatment Liquors

For analysis by SEC or LC-MS, pretreatment liquors at alkaline pH were filtered through a 0.22 μm mixed cellulose ester membrane filter (EMD Millipore, Billerica, Mass.). SEC analysis was performed using an Agilent 1100 HPLC equipped with an Ultrahydrogel 250 column (Waters, Milford, Mass., USA) as described in Stoklosa R J, Hodge D B: Extraction, recovery, and characterization of hardwood and grass hemicelluloses for integration into biorefining processes. Ind Eng Chem Res 2012, 51:11045-11053. Aqueous solutions of monodisperse sodium polystyrene sulfonate (Sigma-Aldrich, St. Louis, Mo., USA) of known molar mass (2000, 4300, 6800, 10000, 32000, and 77000 Da) were used as calibration standards.

Samples for Liquid Chromatography-Mass Spectrometry (LC-MS) analysis were prepared as described above for SEC analysis, except that the concentrations of CuSO4 and 2,2′-bipyridine during pretreatment were 2 mM and 4 mM, respectively. For LC-MS analysis, 10 μL of undiluted pretreatment liquor sample were injected into a XEVO G2SQTOF mass spectrometer in combination with a Waters Acquity® UPLC system and equipped with an Electrospray Ionization (ESI) interface capable of operating in both positive- and negative-ion modes. Chromatographic separation was carried out on a Thermo Beta Basic 100×2.1 mm C18 column (Thermo Fisher Scientific, Waltham, Mass., USA) maintained at 40° C. The binary solvent gradient consisted of 0.1% formic acid in water (solvent A) and 100% methanol (solvent B) in the following gradient: 95% solvent A for the first 3 min, 50% solvent A over the next 1 min, 30% solvent A over next 2 min, and 5% solvent A over the final 2 min. The column was then returned to 95% solvent A, and equilibrated for 2 min prior to the next injection. A solvent delay of 2 min was used to prevent saturation of the detector with the sample solvent.

The negative-ion mode mass spectrometry conditions were constant during all experiments with a voltage of −2.25 kV and a desolvation temperature of 350° C. Mass Lynx software (Waters) version 4.1 was used for system control and data acquisition. The raw data acquired were processed using the Target Lynx application. Pure standards for vanillin, vanillic acid, acetovanillone, syringaldehyde, syringic acid, acetosyringone, and p-hydroxybenzoate (Sigma-Aldrich, St. Louis, Mo., USA) were used to validate peak compound identification and for quantitation.

For quantitation of p-hydroxybenzoate, samples were prepared following the same procedure as that for the LC-MS analysis, but the samples were then analyzed via high-performance liquid chromatography (Agilent 1260 LC equipped with an Agilent Poroshell 120 EC-C18 column (4.6×50 mm) and a diode array detector (DAD). Integration of the p-hydroxybenzoate peak at 280 nm and comparison to a standard curve was used for quantitation. A binary isocratic solvent system was utilized consisting of 80:20 solvent C to solvent D, where solvent C is acetonitrile with 0.1% water, and solvent D is acetonitrile with 0.1% trifluoroacetic acid.

### NMR Analysis of Whole Cell Walls and Pretreatment-Solubilized Lignin

Following the pretreatment, the aqueous phase was separated from the solid phase (i.e., the insoluble portion of pretreated poplar) via filtration, and the filtrate was acidified to pH 2.0 with 72% (w/w) sulfuric acid. The precipitate from the acidified filtrate was recovered via centrifugation and washed with a large volume of aqueous sulfuric acid (pH 2.0) followed by a final washing step of re-suspending and decanting the lignin sample in pH-neutral deionized water. The washed lignin precipitate was lyophilized prior to NMR analyses. The 2D HSQC NMR spectra of three types of samples (untreated hybrid poplar, recovered solubilized lignins and the insoluble portion of pretreated poplar) were acquired and analyzed according to the methods described in Kim H, Ralph J, Akiyama T: Solution-state 2D NMR of ball-milled plant cell wall gels in DMSO-d₆. Bioenerg Res 2008, 1:56-66 (hereinafter “Kim 2008”) Untreated and pretreated samples were prepared for gel-state NMR as described in Kim (2008).

In brief, the dried sample was pre-ground for 1 min in a Retsch MM400 mixer mill at 30 Hz, using zirconium dioxide (ZrO2) vessels (10 mL) containing ZrO₂ ball bearings (2×10 mm). The ground material was extracted with distilled water (1 hr, 3 times) and 80% of ethanol (1 hr, 3 times) with ultrasonication. The cell walls were dried and finely ball-milled using a PULVERISETTE 7 (Fritsch, Idar-Oberstein, Germany) mill at 600 rpm with ZrO₂ vessels (50 mL) containing with ZrO₂ ball bearings (10×10 mm). Each sample (200 mg) was milled for 1 hr, 40 min in 10 min intervals with 5 min interval breaks. The ball-milled samples (50 mg of each) were transferred into 5-mm NMR tubes and gels formed using DMSO-d₆/pyridine-d₅ (4:1, v/v, 0.5 mL) with sonication (30 min). NMR spectra were acquired on a Bruker BioSpin (Billerica, Mass., USA) AVANCE 700 MHz spectrometer equipped with a cryogenically-cooled 5-mm triple-resonance 1H/13C/15N TXI gradient probe with inverse geometry (1H coils closest to the sample).

The central DMSO solvent peak was used as an internal reference (δ_{c} 39.5, δ_{H} 2.49 ppm). The ¹H-¹³C correlation experiment was an adiabatic HSQC experiment (Bruker standard pulse sequence ‘hsqcetgpsisp.2’; phase-sensitive gradient-edited 2D HSQC using adiabatic pulses for inversion and refocusing). HSQC experiments were carried out using the following parameters: acquired from 9 to 1 ppm in F2 (1H) with 1,200 data points (acquisition time 200 ms), 160 to 10 ppm in F1 (13C) with 512 increments (F1 acquisition time 13.6 ms) of 32 scans with a one-second interscan delay; the d24 delay was set to 0.86 ms (⅛J, J=145 Hz).

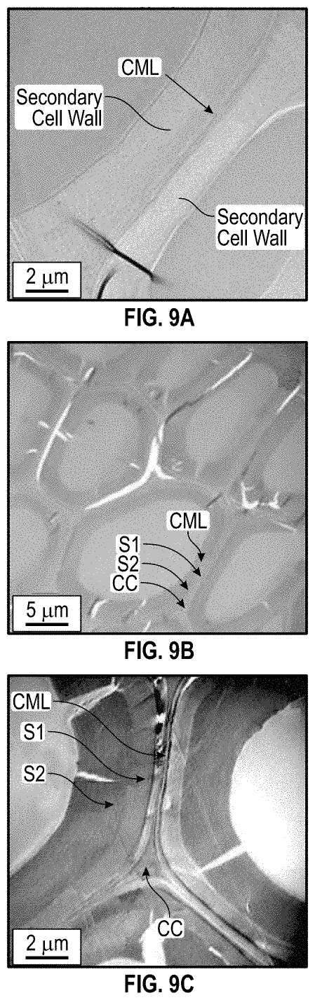

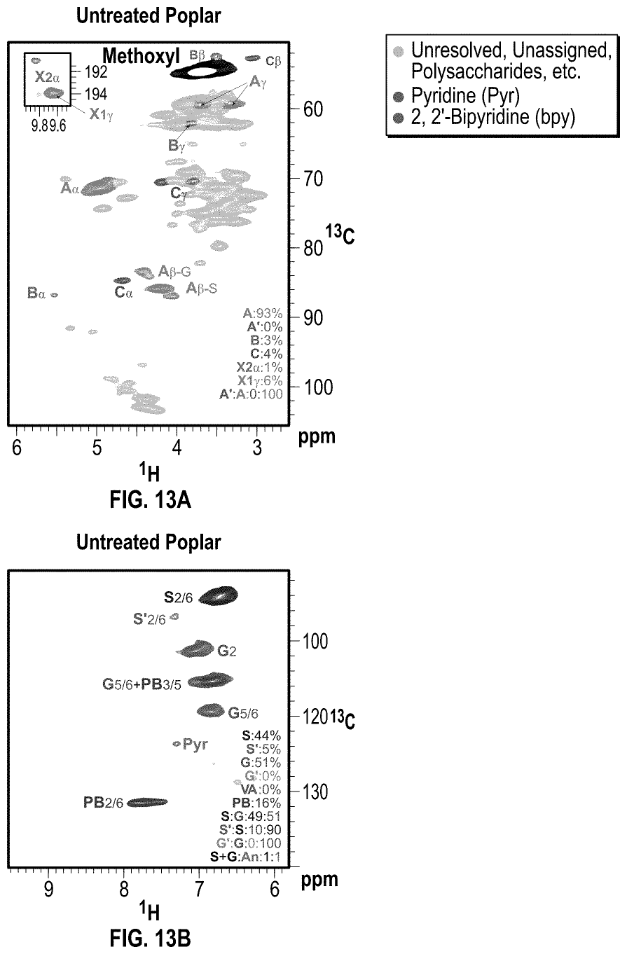

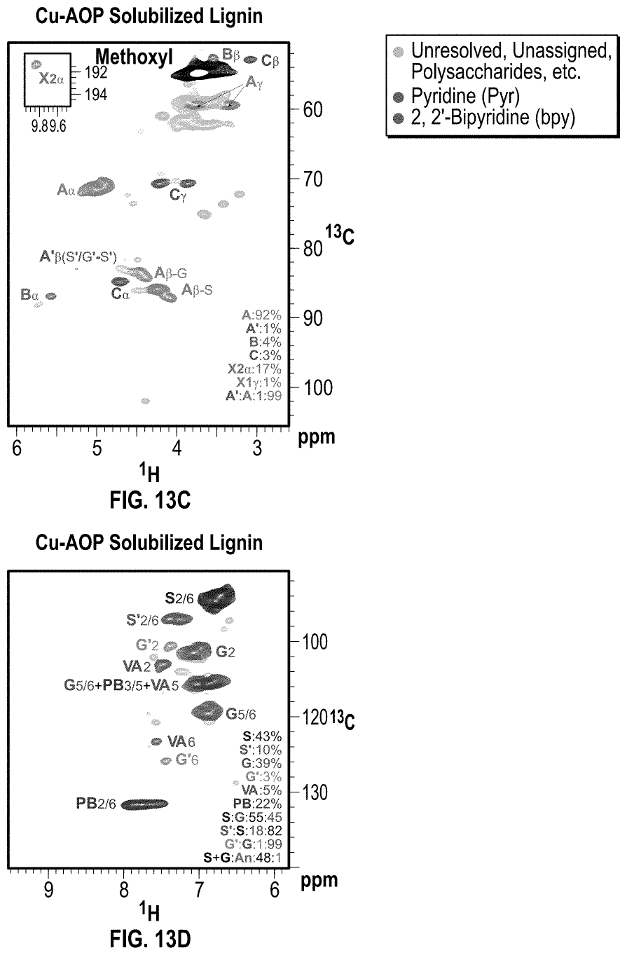

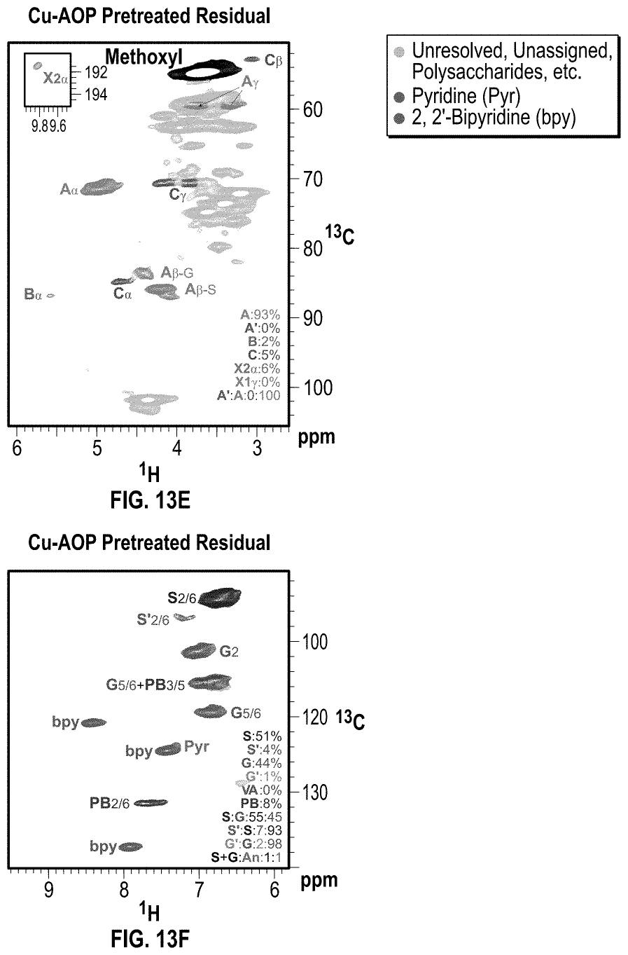

Volume integration of contours in HSQC plots used Bruker's TopSpin 3.1 (Mac) software. Assignments of peaks from NMR spectra were based on Kim 2008 and Kim H, Ralph J: Solution-state 2D NMR of ball-milled plant cell wall gels in DMSO-d6/pyridine-d5. Org Biomol Chem 2010, 8:576-591. TEM imaging was used to characterize corn stover cell wall structural changes associated with pretreatments by dilute acid, and anhydrous ammonia, as well as acid chlorite delignification. The TEM micrographs of untreated hybrid poplar (FIG. 9A) and AOP-only pretreated hybrid poplar (FIGS. 9B and 9C) showed identifiable features of the cell walls comprising wall layers that include secondary cell wall layers (FIGS. 13A-13F and FIG. 15) and the compound middle lamella (CML), as well as cell corners (CC) and individual lumens.

Following AOP-only pretreatment (FIGS. 9A and 9B), the cell walls retained much of their structural integrity, as seen by the similarities to those of untreated cell walls. The only notable changes were dislocations which formed between the middle lamellae and the primary cell walls, possibly due to removal of some lignin and pectic polysaccharides during pretreatment. The dark black stripes observed in the micrographs are artifacts introduced during ultramicrotome sectioning and KMnO₄ staining.

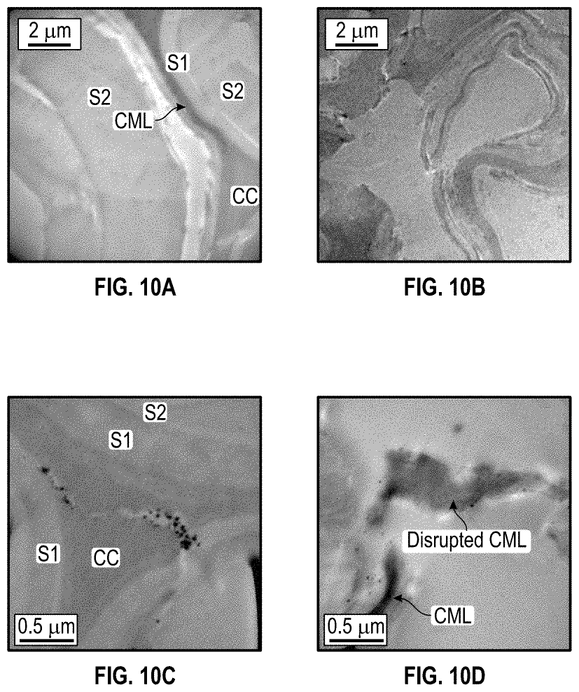

Addition of Cu(bpy) complexes during AOP pretreatment of hybrid poplar improved delignification and enzymatic hydrolysis yields. TEM characterizations of hybrid poplar cell wall after Cu-catalyzed AOP pretreatment are shown in FIGS. 10A-10D, with FIG. 10A showing delamination, FIG. 10B showing dislocations of cell wall layers along, and FIGS. 10C and 10D showing the accumulation of nanoparticles in disrupted regions. “S1” and “S2” indicate different layers of the secondary cell wall; “CC” refers to a cell corner and “CML” refers to the compound middle lamella. As FIGS. 10A-10D show, significant cell wall structural changes occurred as a result of Cu(bpy)-catalyzed AOP pretreatment. See, for example, the major dislocations in the cell wall in FIG. 10A, together with formation of fractures in which the secondary cell wall layers were perturbed. Fractures and disruptions were also observed in other lignin-rich regions, including cell corners (FIG. 10A) and compound middle lamellae (FIG. 10B), suggesting that the structural changes may be caused by lignin modification and/or removal.

The testing described in this example was performed at temperatures ranging from about 25 to about 30° C., which is well below the lignin glass transition (which is about 100 to about 170° C.). As a consequence, the lignin removal which occurred is primarily due to chemical modification and/or solvent effects rather than thermal effects. Additionally, and as shown in FIGS. 10C and 10D, X-ray-opaque particles with diameters in the range of about 20 to about 100 nm were often co-localized with the modified regions of cell walls. Interestingly, such particles were not found in untreated hybrid poplar (FIG. 9A) or AOP-only pretreated poplar (FIGS. 9B and 9C). As such, the development of such particles can be hypothesized to originate from the copper catalyst, rather than as artifacts introduced during the TEM sample preparation. This suggests that copper is involved in the observed cell wall modification (e.g., via lignin oxidation).

### Elemental Profiling of Pretreated Cell Walls

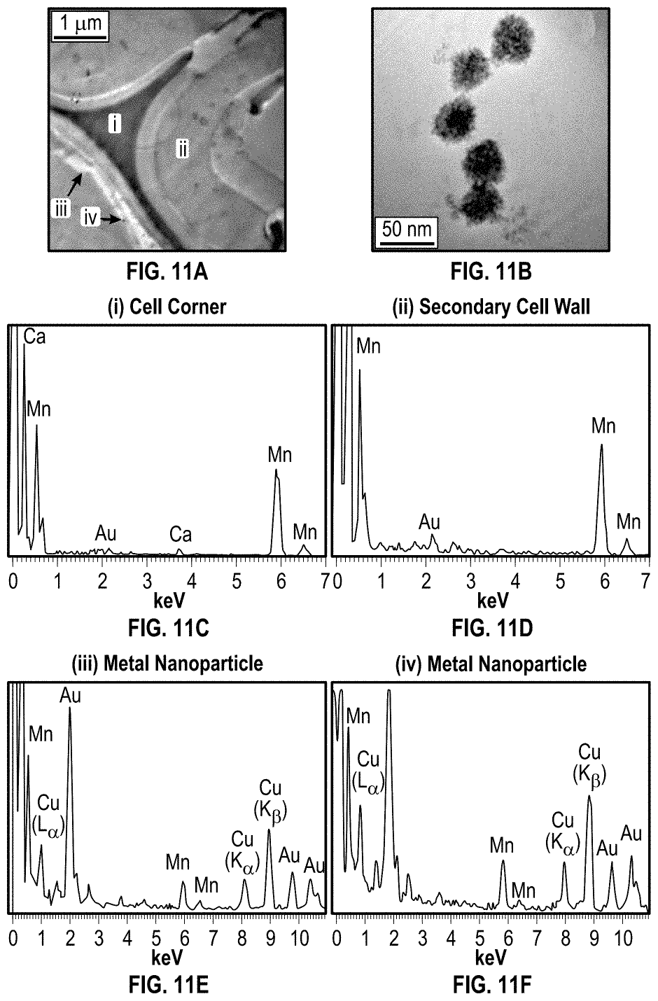

When combined with electron microscopy, in situ elemental profiling using energy-dispersive X-ray spectroscopy (EDS) and electron energy-loss spectroscopy (EELS) can provide chemical characterization at a spatial resolution on the order of 100 nm. To characterize the elemental composition of the nano-scale particles observed in the TEM images, EDS spectra were acquired at different locations in a TEM sample (FIG. 11A), including at a cell corner (identified as area “i” in FIG. 11A), within a secondary cell wall (area “ii”), and over the previously described particles (areas “iii” and “iv”).

TEM images at high magnification show that these particles are aggregates with dendritic structures and diameters on the order of 50 nm (FIG. 11B). A comparison of the EDS spectra reveals both similarities and differences in elemental composition of the cell wall regions (FIGS. 11C-11F). Mn peaks are apparent in all four spectra and are a consequence of the permanganate staining, whereas the Au peaks correspond to the X-ray emissions from the grid that supports the TEM sample. The EDS spectrum from the cell corner (area “i”) shows a strong Ca L-edge peak indicating the presence of Ca ions, which are known to complex with pectin. Ca K-edge peaks (3.7 keV) are also present at a lower relative abundance in the spectra of the other cell areas. In the areas of “ii” and “iii” where X-ray-opaque particles were analyzed, the EDS spectra feature characteristic peaks for Cu. The Cu L-edge and K-edge peaks are not seen in the EDS spectra of either the cell corner (area “i”) or the secondary cell wall (area “ii”). EELS was used to identify the oxidation state of the Cu-containing particles (FIG. 15), which shows the spectrum of a Cu-containing particle with the pre-edge background subtracted. The white-line intensity (i.e., the sharp threshold peaks) of the Cu L2,3 edge indicates that the majority of the Cu is in the Cu(I) oxidation state, while the relatively low white-line intensity of L3 implies that Cu(0) is also present.

The identification of Cu-containing particles suggests that the Cu catalyst is localized in the cell wall matrix at sites corresponding to those with significant structural modification. This result suggests that the soluble Cu catalysts diffuse into the porous cell wall matrix during pretreatment and subsequently catalyze the formation of localized reactive oxygen species that may be involved in the oxidative delignification and structural modification of the cell wall in their vicinity. Although the active catalytic complexes have not yet been identified, one possibility is that the oxidation reactions were accelerated by Cu complexes that catalyzed the decomposition of H₂O₂ and/or activated H₂O₂ via the formation of Cu-peroxide complexes.

With respect to the timing of the formation of the Cu-containing particles, it is possible that the solubility and speciation of the Cu(bpy) complexes are a function of pH, concentration, and ligand to metal ratio, with the Cu(bpy) complexes being substantially more soluble at the alkaline pH where pretreatment occurs. As a consequence, Cu-containing particles may be precipitating at the neutral pH where sample fixation is performed. Another possibility is that the observed Cu-containing particles are providing indirect evidence of catalytic activity as the soluble Cu(bpy) complexes are reduced from Cu(II) to the observed Cu(I) and Cu(0) oxidation states during the pretreatment process and are subsequently deposited as insoluble aggregate particles. It is not yet known whether or not these particles are catalytically active or inactive. Furthermore, the reduction of Cu(II) by the incident electrons during TEM imaging also cannot yet be ruled out.

### Characterization of Solubilized Cell Wall Biopolymers and Phenolic Monomers

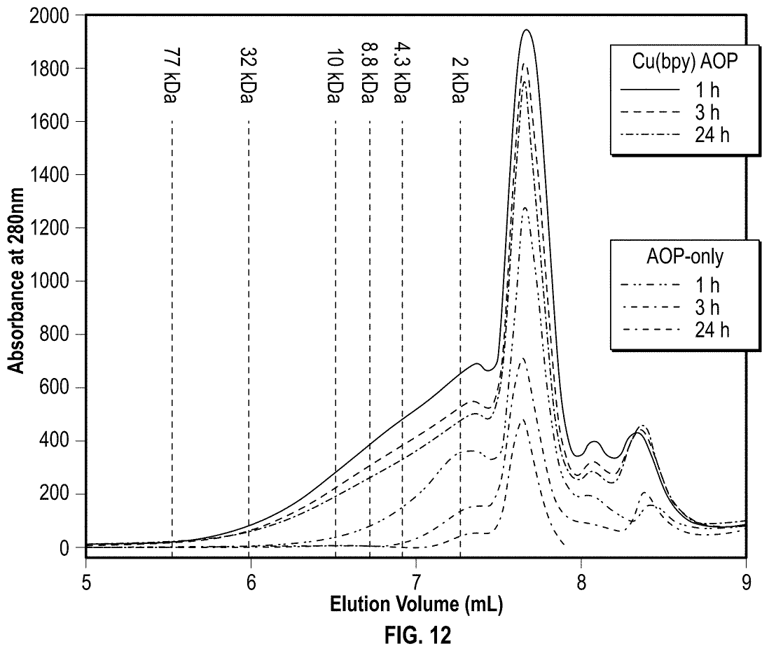

Cell wall biopolymer structural changes associated with this pretreatment were assessed using multiple analytical approaches. In the first approach, the relative abundance and molecular weight distributions of the lignin solubilized during pretreatment were determined by size-exclusion chromatography (SEC) as a function of pretreatment time (FIG. 12).

As can be seen in FIG. 12, a single large peak representing phenolic monomers and oligomers eluted below an apparent molecular weight of 1000 Da. As these elution profiles show, Cu-catalyzed AOP released significantly more soluble lignin fragments than AOP-only. Additionally, as FIG. 12 shows, the molecular weight distributions of the solubilized lignins were not noticeably altered for either of the pretreatments over time, indicating that the soluble lignins were neither undergoing substantial depolymerization, nor repolymerization through condensation reactions.

The distribution and abundance of the phenolic monomers solubilized following pretreatment were quantitated by LC-MS and HPLC. These monomers arise primarily through the cleavage of ether bonds or by saponification of phenolic acids that acylate the lignin polymer. The distribution of phenolic monomers was found to be substantially different when the Cu catalyst was present, with aldehyde products favored over acids as shown in FIG. 4A.

This quantitative difference between the release of phenolic acids and aldehydes suggests that the Cu-catalyzed reaction may utilize a different reaction mechanism that results in less oxidation of the lignin polymer, yet yields more intra-lignin bond cleavage and lignin solubilization than AOP-only treatment.

Additionally, phenolic monomer yields reached only 2 mg/g lignin (i.e., mass of monomer to mass of cell wall lignin) for both AOP-only and Cu-catalyzed AOP pretreatment (FIG. 4.), and only 0.5 mg/g lignin by alkali-only pretreatment (FIG. 4A). T Such low phenolic monomer yields suggest that the cleavage of β-O-4-bonds is not complete.

Plants within the family Salicaceae, including the genus Populus, are known to have lignins with p-hydroxybenzoate groups acylating the γ-OH of syringyl subunits. As expected, the most abundant phenolic monomer in all of the pretreatment liquors was p-hydroxybenzoate (FIG. 4B), as these esters are easily saponified during alkaline pretreatments. The results show that alkali-only treatment results in the highest yields (14.8 mg/g lignin) with AOP-only and Cu(bpy)-AOP, releasing roughly one third or one half of this quantity. The lower yields following the oxidative treatments are presumably due to the lower pHs during these treatments (due to the acidic contribution of H₂O₂) that may result in incomplete saponification of the p-hydroxybenzoate, although oxidative degradation/modification may also contribute to this difference.

### 2D HSQC NMR of Whole Cell Walls and Solubilized Lignins

2D HSQC (heteronuclear single-quantum coherence) NMR spectroscopy was used to analyze lignin after Cu-catalyzed AOP pretreatment. NMR characterization was performed on various components, including untreated whole cell wall material from hybrid poplar (FIGS. 13A and 13B), the lignin that was solubilized following one hour of catalytic pretreatment and recovered via acid precipitation (FIGS. 13C and 13D), and the residual insoluble cell wall material following pretreatment (FIGS. 13E and 13F). Several important insights can be gained from these experiments. First, the NMR data provide evidence for lignin oxidation. The aromatic region (FIG. 13D) revealed a substantial increase in oxidized S and G units to their benzylic ketone analogues S′ and G′ plus new vanillate units (VA). The aliphatic region (FIG. 13C) further supports these chemical changes. Contours are colored to match the structures for aromatic components.

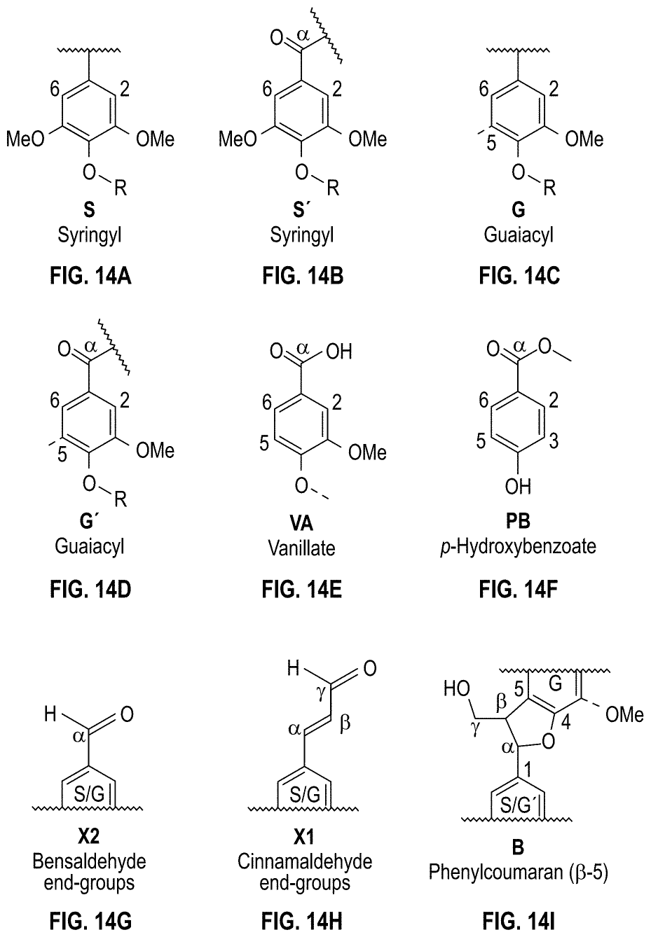

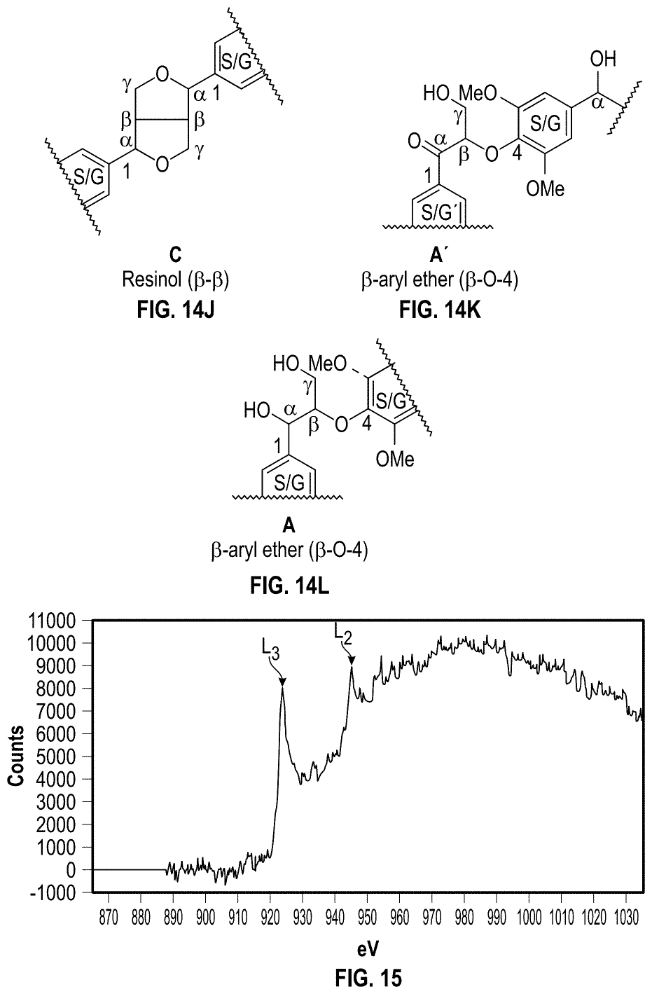

Although most of the correlations corresponding to β-ether units in the aliphatic region remained intact, correlations for the corresponding oxidized analogues A′ provide further evidence of benzylic oxidation. It is not fully understood how other lignin structures, such as β-5-linked units (phenylcoumaran), and β-β-linked (resinol) units (See FIGS. 14I and 14J), react. However, such structures remained intact in this fraction. It was also observed that cinnamaldehyde end groups (X1) were completely absent from the oxidized lignin samples, whereas benzaldehyde end groups (X2) remained. Monomeric and oligomeric fragments with aryl-aldehyde and aryl-acid structures are known to be present in milled wood lignins following catalytic oxidation, and also in lignosulfonates (i.e., lignins derived from acid sulfite pulping of wood). Although the aromatic ring is inactivated toward oxidation due to carbonyl conjugation, the aryl α-carbonyl is susceptible to nucleophilic attack by hydroxyl groups followed by cleavage of the side-chain C_{α}-C_{β} bond. Such cleavage decreases the molecular weight of polymeric lignin and creates hydrophilic lignin fragments with benzoate and benzaldehyde end-groups, consistent with the MS data (FIG. 4A).

It was also observed that lignin depolymerization was not extensive for Cu-catalyzed alkaline oxidative pretreatment (Cu-AOP). Using known peak integration methods, it was observed that the β-O-4, β-5 and β-β linkages were still present in both the solubilized lignin (FIG. 13C) and in the residual pretreatment-insoluble lignin (FIG. 13E) in approximately the same ratio as in the native lignin. Together with the low yields of aromatic monomers observed in LC-MS (FIG. 4A) and the SEC studies performed in this example that revealed an increase in solubilized lignins without a noticeable shift in the molecular weight distributions (FIG. 12), these NMR results strongly support a pathway in which Cu-AOP process solubilizes and removes a fraction of the cell wall-bound lignin with minimal depolymerization and minimal oxidation of the residual lignin.

The solubilized lignins (FIGS. 13C and 13D) showed minimal depolymerization, exhibited minor oxidative modification, and contain soluble xylan oligosaccharides (not shown) unless these are further hydrolyzed and converted. The residual, pretreatment-insoluble lignin (FIGS. 13E and 13F) exhibited minimal modification as a consequence of the pretreatment with structures closely resembling native lignins (albeit requiring recovery of bipyridine). Furthermore, by controlling the pretreatment time and the oxidation stoichiometry, it might be possible to control the molecular weight and chemical properties of solubilized lignins, customizing them for the production of functional materials and fine chemicals with targeted properties.

### Conclusions

These results provide insights into the structural changes that occur to the cell wall and cell wall biopolymers following Cu-catalyzed alkaline oxidative pretreatment (Cu-AOP) of hybrid poplar. Specifically, the catalyzed pretreatment resulted in disrupted cell walls manifested by dislocations between individual cell walls, as well as delaminations within cell walls. Additionally, copper-containing nanoparticles are co-localized within these zones of disruption.

It is hypothesized that sorption of catalyst into the cell wall during pretreatment results in oxidation, solubilization, and removal of lignin causing observable cell wall disruptions and enhanced susceptibility to enzymatic hydrolysis. Consistent with this hypothesis, both LC-MS and NMR characterization of the solubilized lignins and the residual material following Cu-catalyzed AOP pretreatment revealed the presence of oxidized lignin fragments. Specifically, a fraction of the hydroxyl groups at the α-carbon in β-O-4-units were oxidized to carbonyls, and end-groups characteristic of hydrolytic cleavage of oxidized lignin side-chains were created, suggesting that depolymerization results in lignin solubilization and removal during the pretreatment. Whereas the pretreatment-solubilized lignins exhibited a more than three-fold increase in the oxidation of the benzylic alcohol relative to native lignin (with correlation peak integrals increasing from 5% to 18%), the extent of lignin oxidation was limited in the pretreatment-insoluble lignin, which resembled native lignins.

Formation of the Cu-containing nanoparticles with oxidation states of Cu(I) and Cu(0) lower may be attributed to reduction of soluble Cu(bpy) complexes during pretreatment, although it is possible that these particles were formed during sample preparation. Additionally, relative to the lignins generated during other pretreatments and/or delignification processes that were performed at elevated temperatures, substantial lignin modification was often the result. Furthermore, the mildly oxidized lignins generated retained features closely resembling native lignins, which may provide added value to an integrated biorefining process.

### Example 11

### Biomass and Compositional Analysis

Hybrid poplar (Populus nigra var. charkoviensis x caudina cv. NE-19) grown at the University of Wisconsin Arlington Agricultural Research Station was milled to pass through a 1 mm screen (Circ-U-Flow model 18-7-300, Schutte-Buffalo Hammermill, LLC). The initial composition of structural carbohydrates and acid-insoluble lignin (Klason lignin) of biomass were determined using the NREL two-stage acidolysis method (See Sluiter A. et al., Determination of Structural Carbohydrates and Lignin in Biomass. National Renewable Energy Laboratory, NREL/TP-510-42618, Golden, Colo., 2012).

### Catalytic Copper-Catalyzed Alkaline Oxidative Pretreatment (Cu-AOP)

Biomass 0.51 g (about 0.5 g dry basis; from about 3 to about 5% moisture content) was pretreated in a total of 5 mL aqueous solution (10% solids loading). The standard Cu-AOP pretreatment reaction was carried out by adding 4330 μL of distilled water followed by 270 μL 5 M NaOH (100 mg/g biomass), 125 μL of a 40 mM CuSO₄ solution, and 125 μL of a solution containing both 40 mM CuSO₄ and 160 mM 2,2-bipyridine (bpy) (2 mM Cu²⁺ and 4 mM bpy final concentration) and finally 150 μL of 30% H₂O₂ (w/w) (100 mg/g biomass) to the biomass. Reactants were briefly vortexed and the slurry was incubated with orbital shaking at 180 rpm and 30° C. for 24 hrs. The initial pH for the Cu-AOP pretreatment reaction was approximately 11.5.

“Slow Addition of Hydrogen Peroxide” (SH) with Cu-AOP (SH/Cu-AOP)

SH/Cu-AOP pretreatment was performed as described above for standard Cu-AOP except that the 150 μL 30% H₂O₂ (w/w) (100 mg/g biomass) was added to the reaction mixture slowly over a 10-hr period. Specifically, each hour 15 μL of 30% H₂O₂ was added to the reaction mixture, followed by brief mixing in a vortex mixer to ensure an even distribution. Following the final addition of H₂O₂, the mixture was incubated as described for an additional 14 hrs (24 hrs total reaction time).

Alkali Pre-Extraction (AP) with Cu-AOP Pretreatment (AP/Cu-AOP)

Alkali pre-extracted hybrid poplar was prepared by incubating 0.51 g (approximately 0.5 g dry basis) of biomass in 270 μL of 5 M NaOH (100 mg/g biomass) at 30° C. for 1 hr, (10% solids loading). After 1 hour of incubation, the remaining insoluble biomass was washed with one volume of deionized water and subjected to 23 hrs of Cu-AOP pretreatment (24 hrs total reaction time including the 1-hr pretreatment) as described above.

Combined AP and SH with Cu-AOP (AP-SH/Cu-AOP)

Alkali pre-extraction and slow H₂O₂ addition strategies were combined in the modified Cu-AOP (AP-SH/Cu-AOP). Alkali pre-extracted biomass (0.51 g or 0.5 g dry weight) was subjected to SH/Cu-AOP as described above except that the concentration of the Cu(bpy) complexes was reduced by 50% (i.e. 1 mM Cu and 2 mM bpy total final concentrations). Whenever a different concentration of any of the reactants was used to probe the effect on the pretreatment process, an appropriate amount of distilled water was added to the reaction mixture to maintain a final solid biomass loading of 10%.

### Enzymatic Hydrolysis

Following pretreatment, 0.5 mL of 1 M citric acid buffer (pH 5) was added to the pretreatment mixture, and the slurry was slowly titrated with 72% (w/w) H₂SO₄ to adjust the pH to 5 prior to enzymatic hydrolysis. An enzyme cocktail consisting of Cellic CTec3 and HTec3 (kindly provided by Novozymes A/S, Bagsværd, DK) at a loading of 30 mg protein/g glucan each unless otherwise noted was added to the hydrolysis reaction. The protein concentrations of the stock enzyme cocktails were quantified by determining the total protein concentration (and subtracting the non-protein nitrogen contribution) using the Kjeldahl nitrogen analysis method (AOAC Method 2001.11, Dairy One Cooperative Inc., Ithaca, N.Y., USA). The total volume of the enzymatic hydrolysis reaction was then adjusted to 10 mL by the addition of deionized water, and the samples were incubated at 50° C. for 72 hrs with orbital shaking at 210 rpm. Following enzymatic hydrolysis, the solid and liquid phases were separated by centrifugation, and the amount of glucose and xylose released into the aqueous phase was quantified by HPLC (Agilent 1260 Series equipped with an Agilent 1260 infinity refractive index detector) using an Aminex® HPX-87H column operating at 65° C., a mobile phase of 0.05 M H₂SO₄, and a flow rate of 0.6 mL/min. Standard curves using pure glucose and xylose were prepared prior to each analysis to convert peak area to concentration of monomeric sugar. The yield of glucose and xylose released was defined as the amount of solubilized monosaccharide divided by the total sugar content of the biomass prior to pretreatment as determined by chemical composition analysis. The error bars in the figures represent the standard deviation from three or more biological replicates.

### Results and Discussion

Slow Addition of H₂O₂(SH/Cu-AOP)

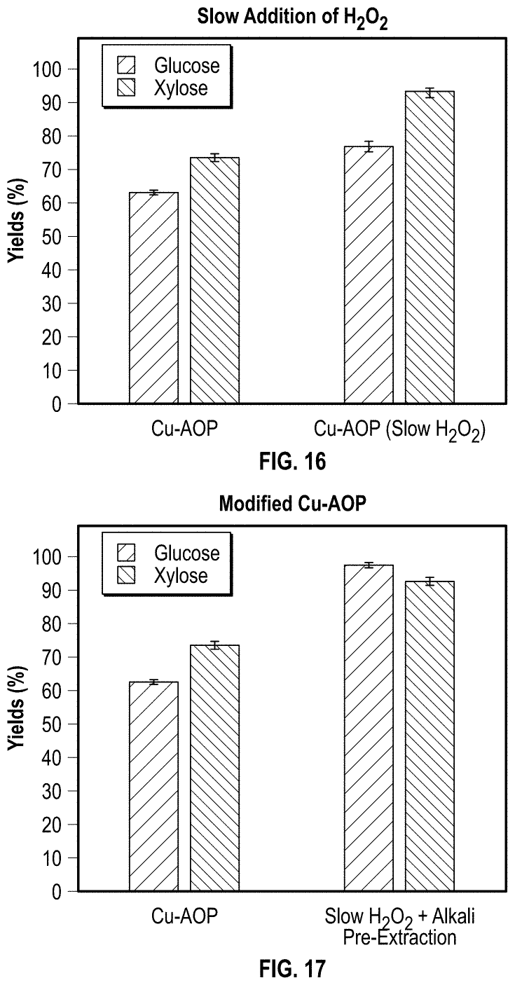

It was hypothesized that by lowering the effective H₂O₂ concentration during AOP, a decrease in non-productive reactions would occur, thus enabling more of the reactive oxygen species to react with the biomass. To test this hypothesis, the H₂O₂ was added in a “slow add” manner. Specifically, the H₂O₂ was added over the course of 10 hrs without changing either the total amount of peroxide utilized or the pretreatment time (SH/Cu-AOP). Relative to Cu-AOP conditions, this modification of the rate of addition of oxidant resulted in a greater than 1-fold increase in sugar yields, namely an approximately 1.2-fold increase (77% glucose and 93% xylose) following enzymatic hydrolysis (See FIG. 16 and Table 3 below).

TABLE 3
Sugar yields obtained from different pretreatment strategies

| Method | Glucose yields (%) | Xylose yields (%) |
|---|---|---|
| Cu-AOP | 62 | 75 |
| AP/Cu-AOP | 86 | 95 |
| SH/Cu-AOP | 77 | 93 |
| AP-SH/Cu-AOP | 97 | 94 |

To ascertain how SH/Cu-AOP alters the cell wall of hybrid poplar relative to standard Cu-AOP, compositional analysis of the biomass was performed both before and after pretreatment. These results showed that the major impact of slow addition of H₂O₂ was an increase in the extent of lignin removal, with approximately 44% of the original lignin being solubilized during SH/Cu-AOP compared to 28% during Cu-AOP (FIG. 17 showing an increase in the yields of glucose and xylose to 96% and 93%, respectively, (Table 4). As discussed herein, lignin removal is a significant factor in overcoming biomass recalcitrance and improving sugar yields, by improving access to the cellulose. This aspect of lignin removal may help explain why the more efficient utilization of H₂O₂ in SH/Cu-AOP significantly increased the efficacy of pretreatment and subsequently the final sugar yields.

TABLE 4
Percent mass loss obtained different pretreatments

| Pretreatments of | Glucan | Xylan | Lignin |
|---|---|---|---|
| hybrid poplar | loss (%) | loss (%) | loss (%) |
| Untreated | N/A | N/A | N/A |
| Normal Cu-AOP | 3 ± 0.03 | 23 ± 0.01 | 28 ± 0.4 |
| AP | 1 ± 0.9 | 5 ± 0.3 | 5 ± 0.4 |
| AP/Cu-AOP | 5 ± 1.2 | 32 ± 0.3 | 40 ± 1.4 |
| SH/Cu-AOP | 6 ± 0.8 | 23 ± 0.5 | 44 ± 0.00 |
| AP-SH/Cu-AOP | 6 ± 1.5 | 39 ± 0.4 | 56 ± 0.3 |

Lignin: Acid insoluble Klason lignin
Untreated: Untreated hybrid poplar
AP: Alkali pre-extraction only
AP/Cu-AOP: Cu-AOP followed by alkali pre-extraction
SH/Cu-AOP: Cu-AOP with slow addition of H₂O₂
AP-SH/Cu-AOP: Combining alkali pre-extraction and slow addition of H₂O₂ with Cu-AOP

### Alkaline Pre-Extraction Followed by Cu-AOP (AP/Cu-AOP)

Seeking to increase further the efficiency of the pretreatment and to improve the accessibility of the cellulases and hemicellulases to the polymeric sugars, an alkali pre-extraction step was incorporated prior to Cu-AOP (AP/Cu-AOP). By removing easily extracted hemicellulose and lignin, it was thought that the porosity of the biomass would increase, resulting in improved penetration of the Cu(bpy) complexes into the plant cell wall. This, in turn, may lead to efficient activation and utilization of active radical species. In addition, such a step may also remove extractives and lignin to reduce inhibition of enzymes during enzymatic hydrolysis step.

Based on the final sugar yields, the alkaline pre-extraction step had a large positive impact on enzymatic digestibility. Compared to standard Cu-AOP pretreatment of hybrid poplar, AP/Cu-AOP pretreatment resulted in improved glucose yields from 62% to 86% (an approximately 1.4-fold increase), while xylose yields also improved from 75% to 93% (an approximately 1.2-fold increase) (Table 3). Compositional analysis following alkali pre-extraction of hybrid poplar indicated that while approximately 5% by weight of both the lignin and xylan was solubilized during pre-extraction (FIG. 17, Table 4), substantially none or only trace amounts of the glucose was released. Pretreatment of this alkaline pre-extracted poplar by AOP (AP/Cu-AOP) resulted in loss of an additional approximately 35% lignin, which is about 1.4 times higher than Cu-AOP alone. The removal of lignin is synergistic with AP/Cu-AOP, removing nearly 10% more than the sum of AP and Cu-AOP alone, consistent with the significant increase in sugar yields following enzymatic hydrolysis of AP/Cu-AOP poplar (FIG. 2).

Hypothesizing that alkaline pre-extraction aids pretreatment, in part, by preparing the biomass for pretreatment i.e., swelling the biomass and increasing the surface area for enzyme accessibility, the water retention value (WRV) was measured both before and after pre-extraction. Results showed that the WRV of hybrid poplar increased from 1.15 g water/g biomass (untreated) to 1.5 g water/g biomass following the alkaline pre-extraction step, consistent with the importance of WRV to the pretreatment process.

To ascertain if alkaline pre-extraction reduced enzyme inhibition by removing extractives and lignin, the effect of pre-extraction liquor on the hydrolysis of Avicel® was evaluated. These results demonstrated that enzymatic hydrolysis of Avicel® in the presence of alkaline pre-extraction liquor resulted in 6% lower glucose yields compared to hydrolysis reactions that did not contain the pre-extraction liquor. Hydrolysis reactions were also performed in the presence of pretreatment liquors obtained from normal Cu-AHPAOP and AP/Cu-AHPAOP. Interestingly, the inhibitory effect of the Cu-AHPAOP pretreatment liquor was approximately equal to additive inhibitory effect of the AP pre-extraction liquor plus the AP/Cu-AHPAOP pretreatment liquor. Together, these results suggest that pre-extraction of the hybrid poplar with NaOH increases sugar yields by removing alkaline extractable compounds that inhibit enzymatic hydrolysis.

In summary, alkaline pre-extraction prior to Cu-AHPAOP pretreatment (AP/Cu-AHPAOP) results in increased glucose and xylose yields relative to our standard Cu-AHPAOP process, presumably by the removal of alkaline extractable compounds that inhibit enzymatic hydrolysis. These data are consistent with results from Yuan et al. (Bioresour Technol 2013, 144:429-434) demonstrating efficient and synergistic lignin and hemicellulose removal from poplar using a two-step alkali and ionic liquid pretreatment. In addition, Liu et al. (Biotechnol Biofuels 2014, 7:48) recently reported enhanced enzymatic hydrolysis of corn stover by coupling alkaline pre-extraction with alkaline-oxidative post-treatment.

### Combining Alkaline Pre-Extraction and Slow Addition (AP-SH/Cu-AOP)

Having demonstrated that the efficacy of Cu-AOP could be improved dramatically by slowly adding the H₂O₂ over the course of 10 hrs (SH/Cu-AOP), as well as by pre-extracting the poplar with alkali prior to Cu-AOP (AP/Cu-AOP), a combination of the two strategies (AP-SH/Cu-AOP). As shown in Table 3, these two modifications to the Cu-AOP pretreatment of hybrid poplar procedure resulted in the removal of nearly 40% of the xylan and over 55% of the lignin during pretreatment. As expected, this increase in lignin and xylan (hemicellulose) removal resulted in a significant improvement in enzymatic digestibility (FIG. 17). In fact, enzymatic hydrolysis resulted in high sugar yields of 97% and 94% of the theoretical maximum for glucose and xylose, respectively.

This AP-SH/Cu-AOP pretreatment strategy was performed at atmospheric pressure near room temperature. As a result, the AP-SH/Cu-AOP pretreatment can be performed using relatively simple and inexpensive equipment.

### Optimization of AP-SH/Cu-AOP

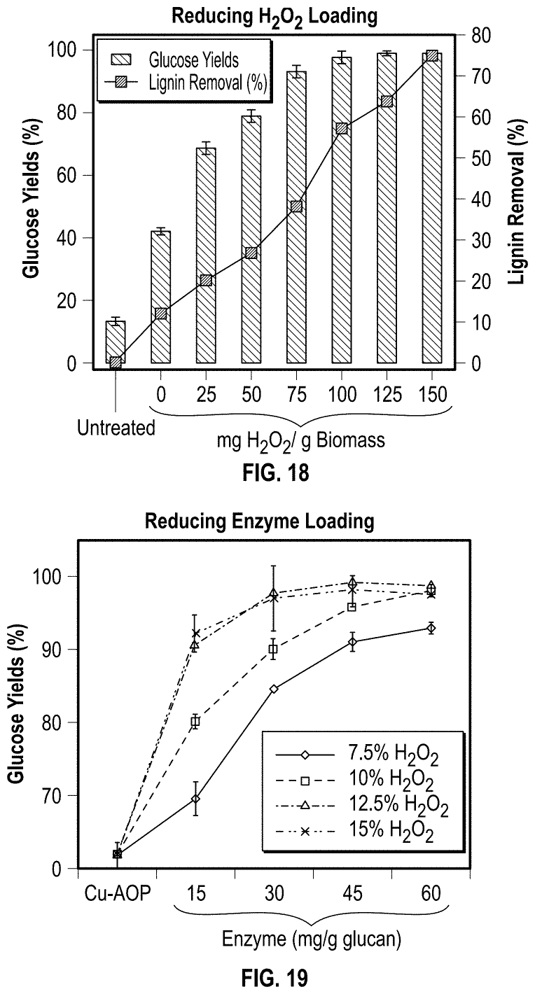

Hypothesizing that these two modifications to the Cu-AOP process might allow a reduction in delignification costs by lowering oxidant loadings, a series of experiments were performed with varying H₂O₂ loadings (FIG. 18). The results demonstrated that the H₂O₂ loading can be reduced by 25% to 75 mg/g poplar while still maintaining sugar yields of over 90%. Even at H₂O₂ loadings as low as 50 mg H₂O₂/g biomass, the sugar yields following enzymatic hydrolysis were higher (78%) than those obtained with normal Cu-AOP which utilizes 100 mg/g biomass (62%).

As expected, compositional analysis of the biomass treated with different H₂O₂ loadings revealed a clear relationship between the extent of lignin removal during AP-SH/Cu-AOP and the sugar yields following enzymatic hydrolysis (FIG. 18). Sugar yields increased rapidly with lignin removal until approximately 40% of the lignin had been removed, at which point additional lignin removal had only a modest effect on sugar yields. This digestibility “threshold” value in between a Klason lignin content of 10-15% may be the point where cell wall loses its enough recalcitrance to allow enzyme accessibility.

The experiments described above were performed utilizing 60 mg protein (enzyme) per g glucan. Hypothesizing that the alkali pre-extraction and the more efficient utilization of H₂O₂ may allow for a reduction in enzyme loading, a series of studies were performed to correlate glucose yields following enzymatic hydrolysis of pretreated hybrid poplar using different enzyme loadings (FIG. 19). Not surprisingly, there was a strong correlation between enzyme loading and sugar yield. As predicted, however, AP-SH/Cu-AOP method resulted in improved sugar yields even at much lower enzyme concentrations. For instance, at 100 mg H₂O₂ per gram poplar (10% H₂O₂), enzyme loadings were reduced to 15 mg/g glucan (a four-fold decrease) with higher glucose yields following enzymatic hydrolysis (approximately 80% versus 60% for our “standard” Cu-AOP conditions). In addition, when the peroxide loadings were increased during pretreatment to 125 mg H₂O₂/g biomass, glucose yields increased to >90% while still utilizing only 15 mg enzyme/mg glucan. This improved saccharification of poplar wood with such low enzyme loadings can be attributed to the relatively high lignin removal during the AP-SH/Cu-AOP process.

As demonstrated in FIG. 19, AP-SH/Cu-AOP at H₂O₂ loadings of 100-150 mg/g biomass removed a substantial amount of lignin from the biomass. This provided an insight of possibility to decrease the concentrations of other costly components involved in Cu-AOP pretreatment, such as bpy.

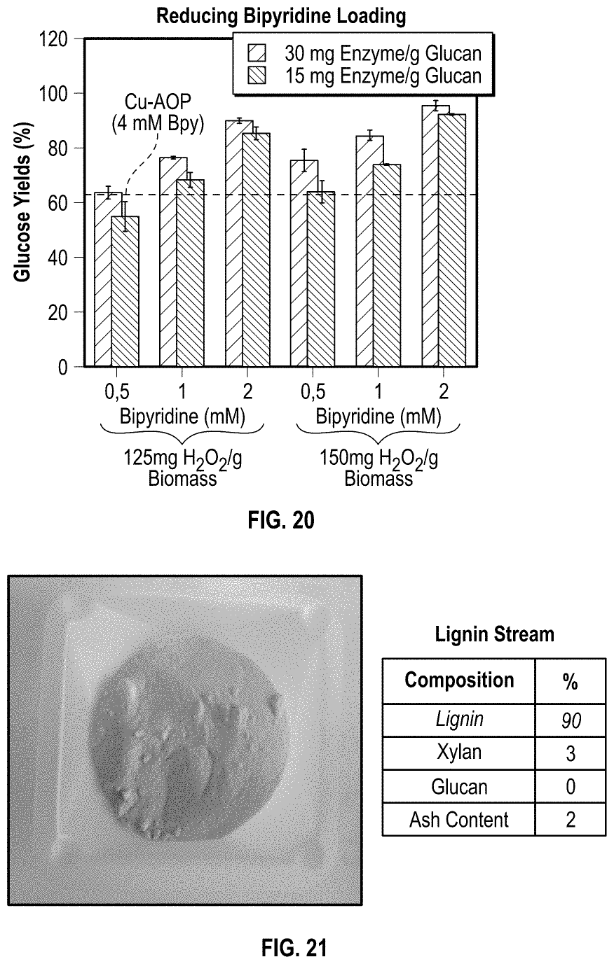

Above discussed strategies for improving pretreatment were performed using 2 mM concentration of bpy. Trials were carried out to select minimum concentration of bpy but still having relatively high sugar yields. FIG. 20 shows sugar yields obtained at different concentrations of bpy, utilizing 125 and 150 mg H₂O₂. Results revealed the potential of low concentrations of bpy at low enzyme loadings of 15 and 30 mg/g glucan. A high glucose yields of 75% were obtained at 0.5 mM concentration of bpy with 30 mg/g glucan enzyme and 150 mg H₂O₂. An increase in the yields to 85% was obtained by increasing the bpy to 1 mM with 30 mg/g glucan enzyme and 150 mg H₂O₂. These results show the potential of AP-SH/Cu-AOP at low bpy loadings. By performing cost analysis of the data, we could figure out the best combinations of the component concentration to lower the cost and maintaining high sugar yields.

### Lignin Stream

As shown in FIG. 19, AP-SH/Cu-AOP solubilized a significant fraction of the total lignin present in hybrid poplar. Recognizing that this delignification process not only improves the enzymatic digestibility of the biomass, but also provides a lignin stream for potential valorization to chemicals and/or fuels, we sought to characterize the solubilized lignin. The solubilized lignin was precipitated at pH 2, washed with deionized water, and lyophilized to dryness. The recovered product contained ≥90% Klason lignin, with only 3% xylan and 2% ash content (See FIG. 21).

### Example 12

Study on Cell Wall Properties of Natural Populus Variants for Enhanced Digestibility with Alkaline Hydrogen Peroxide Pretreatment

In this study, the effect of cell wall redox metal ions, lignin content and S/G ratio on the efficacy of alkaline pretreatments was studied. The results obtained were correlated to digestibility i.e., sugar release from biomass followed by enzymatic hydrolysis.

### Materials and Method

### Biomass

Thirty-six Populus trichocarpa genotypes were provided by Oak Ridge National Laboratory, Oak Ridge, Tenn., USA. Biomass was milled (Wiley, MiniMill, Thomas Scientific, Swedesboro, N.J.) to pass through 20 mesh size screen, air dried, and stored in airtight bags prior to pretreatment studies.

### Biomass Analysis

The initial composition of structural carbohydrates and acid-insoluble lignin (Klason lignin) were determined using the NREL two-stage acidolysis method (See Sluiter A. et al., Determination of Structural Carbohydrates and Lignin in Biomass. National Renewable Energy Laboratory, NREL/TP-510-42618, Golden, Colo., 2012) with modifications as described in Li M Y, Foster C, Kelkar S, Pu Y Q, Holmes D, Ragauskas A, Saffron C M, Hodge D B: Structural characterization of alkaline hydrogen peroxide pretreated grasses exhibiting diverse lignin phenotypes. Biotechnol Biofuels 2012, 5:38. The S/G ratio for all samples were determined by thioacidolysis as described in Li et al., 2012.

Quantification of redox-active metals in different popular species were determined by inductively coupled plasma mass spectrometry (ICP-MS) performed at A & L Great Lakes Laboratories.

### Pretreatment

Each biomass 0.51 g (approximately 0.5 g dry basis; about 3 to about 5% moisture content) was pretreated with three pretreatments viz alkali, alkaline hydrogen peroxide (AOP) and AOP aided with Cu(bpy) catalyst (Cu-AOP) in a total of 5 mL aqueous solution (10% solids loading). For alkali-only pretreatment, the solution contained 50 mg NaOH (100 mg NaOH/g biomass), while AOP pretreatment also contained 150 μL of 30% H₂O₂ (100 mg H₂O₂/g biomass). The Cu-AOP pretreatment solution was prepared as described above for AOP except that 125 μL of a solution containing 40 mM CuSO₄ as well as 125 μL of a solution containing both 40 mM CuSO₄ and 80 mM 2,2′-bipyridine (bpy) were added to the biomass slurry after the addition of NaOH (2 mM Cu²⁺ and 4 mM bpy final concentration) but prior to the addition of H₂O₂. The final pH for the alkali-only pretreatment was 13.2, while it was approximately 11.5 for AOP and Cu-AOP due to the addition of H₂O₂. For all three pretreatments, the reactants were vortexed and the slurry incubated with orbital shaking at 180 rpm at 30° C. Solutions containing only biomass and deionized water acted as the control.

### Enzymatic Hydrolysis

After pretreatment, 0.5 mL of 1 M citric acid buffer (pH 4.8) was added to the pretreated slurry, and the slurry was slowly titrated with 72% (w/w) H₂SO₄ to adjust the pH to 5.0 prior to enzymatic hydrolysis. An enzyme cocktail consisting of Cellic CTec3 and HTec3 (gift from Novozymes A/S, Bagsværd, DK) at a loading of 30 mg protein/g glucan each was added to the hydrolysis reaction. (The protein concentrations of the stock enzyme cocktails were quantified using the Bradford Assay (Sigma-Aldrich)). The total volume was adjusted to 10 mL by the addition of deionized water, and the samples were incubated at 50° C. with orbital shaking at 210 rpm. Following enzymatic hydrolysis, the solid and liquid phases were separated by centrifugation, and the amount of glucose and xylose released into the aqueous phase was quantified by HPLC (Agilent 1260 Series equipped with an Aminex® HPX-87H column operating at 65° C., a mobile phase of 0.05 M H₂SO₄, a flow rate of 0.6 mL/min, and detection by refractive index). The yield of glucose and xylose released was defined as the amount of solubilized monosaccharide divided by the total sugar content of the biomass prior to pretreatment as determined by chemical composition analysis. Although xylan is solubilized during pretreatment, no monomeric sugars were detected in the pretreatment liquor. The error bars in the figures represent the standard deviation from ≥three biological replicates.

Chelation of Inorganic Ions from Native Biomass

Biomass (3 g) was mixed with 30 mL of 0.2% (w/v) of the chelator diethylenetriaminepentaacetic acid (DTPA). The pH of the slurry was adjusted to 7.0 with 5M NaOH, and the solution was incubated for 24 hr at 30° C. The biomass was then washed thoroughly with 10 volumes of distilled H₂O to remove the DTPA, dried at room temperature for 2 days, and stored in airtight bags. Biomass incubated for 24 hr at 30° C. with only distilled water was used as a control.

Pretreatment reactions were performed either in the presence of bpy plus Cu²⁺, Mn²⁺, or Fe²⁺ ([metal]:[bpy]=2 mM: 4 mM) or in the absence of added metal to ascertain the effect these ions had on the pretreatment of chelated woody biomass.

Twenty (20) samples of a natural population of Populus trichocarpa trees were obtained from Oak Ridge National Laboratory, out of which 16 were separated into two fractions individually on the basis of growth, i.e. early growth (EGW) and late growth (LGW) wood samples, which made total number of samples to 32. Lignin content, S/G ratio and cell wall redox metal ions content were quantified to perform correlation studies. Correlations were established between final sugar release after enzymatic hydrolysis and different pretreatments among different poplar samples.

### Results

Sixteen (16) samples out of 20 samples were separated into two fractions on the basis of their growth i.e. early growth wood and late growth wood to study how cell wall properties of different ages of wood relates with wood digestibility. In total, 36 biomass samples were individually pretreated utilizing alkali, alkaline hydrogen peroxide (AOP), and copper catalyzed alkaline hydrogen peroxide (Cu-AOP). To establish digestibility correlation among cell wall properties and different pretreatments, pretreated samples were enzymatically hydrolyzed using commercially available optimized cocktail of cellulases and xylanases. Samples without any pretreatment were also enzymatically hydrolyzed and analyzed.

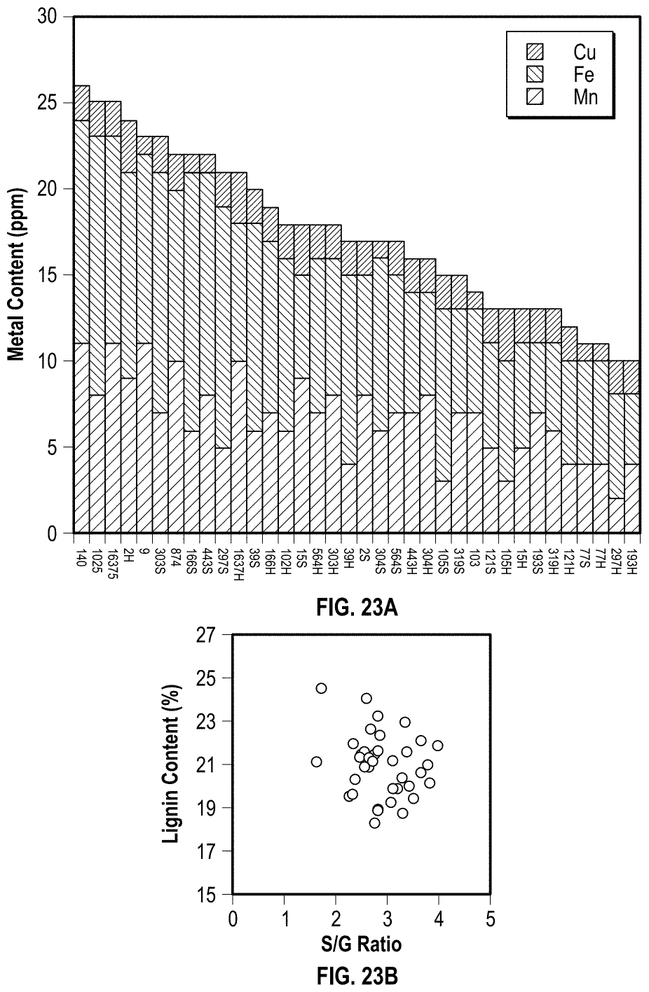

To establish significant correlations, samples were analyzed to determine their lignin content, S/G ratio and total cell wall redox metal ions content. Estimated lignin content was in the range of 18.74 to 24.55%, S/G ratio was 1.62 to 3.98 (FIG. 23B) and metal content was in the range of 10 to 26 ppm (FIG. 23A).

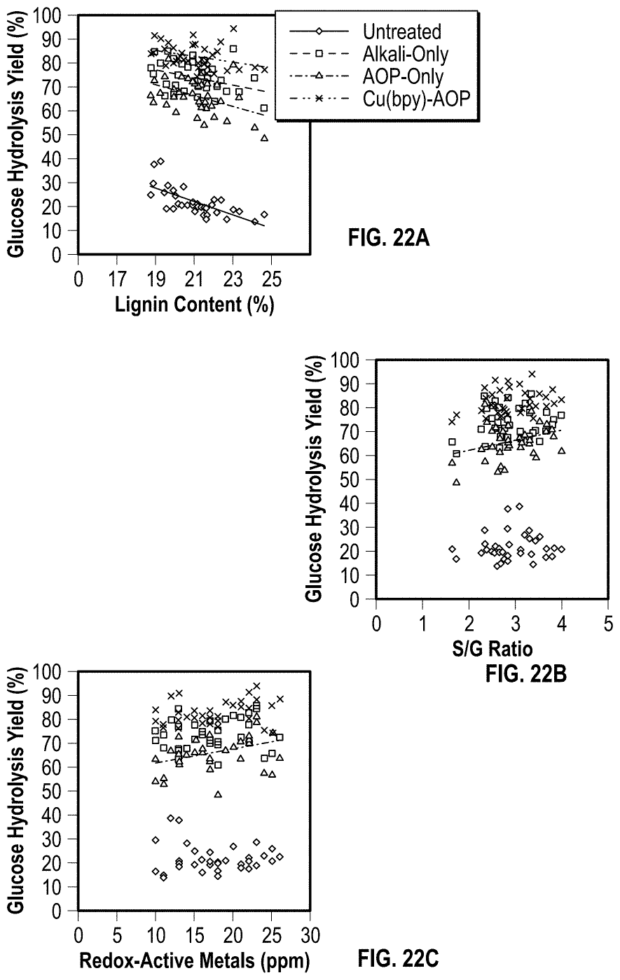

Results demonstrated that for all 36 samples, Cu-AOP pretreatment yielded maximum sugars, followed by yields obtained with AOP, and untreated samples. Sugar yields with untreated samples were in the range from 13 to 38%, 61 to 85% with alkali pretreatment, about 53 to about 81% with AOP pretreatment and 74 to 94% for Cu-AOP pretreatment (FIG. 22).

Specifically, as FIG. 22 shows, there is a negative correlation for lignin content in all pretreatments. Additionally, the S/G ratio correlation strongest for AOP-only treatment (p-value=0.055). The redox-active metal content was only significant for AOP-only treatment.

As demonstrated by the results, addition of copper catalyst led to substantial increase in the sugar yields, these surprising results provided an insight into the role of cell wall redox active metal ions in improving effectiveness of AOP only pretreatment.

A positive correlation was discovered between cell wall redox metal ions and sugar yields when biomass was treated with AOP only. Correlations were insignificant for cell wall metal ions when the biomass was pretreated with alkali and Cu-AOP. The results clearly demonstrated the importance of cell wall redox metal ions in AOP only method. While not wishing to be bound by this proposed hypothesis, it is thought that cell wall metal ions efficiently activated the hydrogen peroxide which catalyzed the formation of highly reactive radical species that aided in the pretreatment process. Correlation between S/G ratio and sugar yields was found to be strongest for AOP pretreated samples compared to alkali and Cu-AOP.

### Chelation of the Biomass

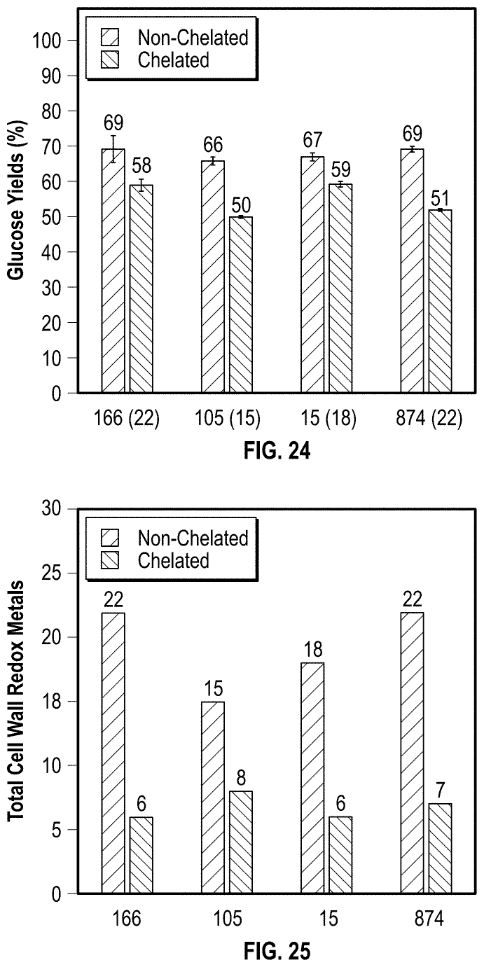

To validate the relationship between intracellular metal content and efficacy of AOP pretreatment, each of the selected four samples was incubated with the metal chelator DTPA to remove the cell wall redox metal ions prior to pretreatment. Results revealed decreased sugar yields with chelated biomass compared to non-chelated biomass (FIG. 24). For example, glucose yields were reduced from 66% to 50% for sample 105. Similar trend was observed for the other biomass samples. To confirm the efficiency of chelation, ICP-MS analysis of the chelated biomass was performed. Results demonstrated reduced cell wall redox metal content compared to non-chelated biomass (FIG. 25).

### Discussion

To further understand the mechanism of AOP pretreatment on woody biomass components, additional studies were carried out.

A total of 36 woody biomass samples were studied to determine the correlation between cell wall properties and fermentable sugar release. Analysis of results revealed that sugar release was negatively correlated to lignin content of the biomass. Lignin serves as a physical blockage on the surface of the biomass and chemical blockage through lignin carbohydrate complex. Adsorption of enzymes on lignin occurs through hydrophobic interactions, electrostatic interactions, and/or hydrogen-bonding interactions, which leads to non-productive binding making cellulose inaccessible to enzymes.

In addition to lignin, lignin composition, i.e., the S/G ratio, was also analyzed, with results confirming the absence of any correlation of S/G rations with the sugar yields. This is in contrast to other reported results, which may be explained by use of a different pretreatment processes.

The effect of total cell wall redox metal ions (Cu, Mn, and Fe) on the digestibility of biomass treated with AOP only. Interestingly, positive correlation was observed between glucose yields and number of metal ions for biomass treated with AOP only pretreatment whereas no correlations were established for alkali, Cu-AOP and untreated samples.

### Example 13

### Biomass

Hybrid poplar (Populus nigra var. charkoviensis x caudina cv. NE-19) was provided by the Great Lakes Bioenergy Research Center (GLBRC), sugar maple (Acer saccharum) was obtained from Todd Smith (Devereur sawmill, Pewamo, Mich.), silver birch (Betula pendula) was acquired from Curt Lindstrom (Smurfit-Kappa Kraftlina AB, Pitea, Sweden) and aspen (Populus tremula x Populous tremuloides) was obtained from Dr. Raymond Miller (Michigan state University Extensions). Biomass was milled using a Wiley MiniMill (Thomas Scientific, Swedesboro, N.J.) to pass through a 20-mesh size screen, air dried, and stored in airtight bags prior to pretreatment studies.

### Compositional Analysis and S/G Ratios

Quantification of structural carbohydrates, acid-insoluble lignin (Klason lignin), cell wall metal content, and S/G ratio were performed as described in Example 12.

### Pretreatment

Three different pretreatment strategies were tested on each of the biomass samples: alkali only, alkaline hydrogen peroxide (AOP), and copper-catalyzed AOP (Cu-AOP). In all cases, 0.51 g of biomass (approximately 0.5 g dry basis; about 3 to about 5% moisture content) was pretreated in a total of 5.0 mL aqueous solution (10% solids loading). For alkali-only pretreatment, the solution contained 50 mg NaOH (100 mg NaOH/g biomass), while AOP pretreatment also contained 150 μL of 30% H₂O₂ (100 mg H₂O₂/g biomass). The Cu-AOP pretreatment solution was prepared as described above for AOP except that 125 μL of a solution containing 40 mM CuSO₄ as well as 125 μL of a solution containing both 40 mM CuSO₄ and 160 mM 2,2-bipyridine (bpy) were added to the biomass slurry after the addition of NaOH (2 mM Cu²⁺ and 4 mM bpy final concentration) but prior to the addition of H₂O₂. The final pH for the alkali-only pretreatment was 13.2, while it was approximately 11.5 for AOP and Cu-AOP due to the addition of H₂O₂. For all three pretreatments, the reactants were mixed in a vortex mixer and the slurry incubated with orbital shaking at 180 rpm and 30° C. for 24 hrs. Solutions containing only biomass and deionized water acted as controls.

### Enzymatic Hydrolysis

Following pretreatment, 0.5 mL of 1 M citric acid buffer (pH 4.8) was added to the pretreated slurry, and the slurry was slowly titrated with 72% (w/w) H₂SO₄ to adjust the pH to 5.0 prior to enzymatic hydrolysis. An enzyme cocktail consisting of Cellic CTec3 and HTec3 (gift from Novozymes A/S, Bagsværd, DK) at a loading of 30 mg protein/g glucan for both Ctec3 and HTec3 was added to the hydrolysis reaction. The protein concentrations of the stock enzyme cocktails were quantified as described in X. Gao, R. Kumar, S. Singh, B. A. Simmons, V. Balan, B. E. Dale and C. E. Wyman, Biotechnol Biofuels, 2014, 7, 71, using the Kjeldahl nitrogen analysis method, as described in, for example, R. Kumar and C. E. Wyman, Enzyme Microb Tech, 2008, 42, 426-433. The total volume of the pretreated biomass slurry was adjusted to 10 mL by the addition of deionized water, and the samples were incubated at 50° C. for 72 hrs with orbital shaking at 210 rpm.

Following enzymatic hydrolysis, the solid and liquid phases were separated by centrifugation, and the amount of glucose and xylose released into the aqueous phase was quantified by HPLC (Agilent 1260 Series equipped with an Aminex® HPX-87H column operating at 65° C., a mobile phase of 0.05 M H₂SO₄, a flow rate of 0.6 mL/min, and detection using an Agilent 1260 infinity refractive index detector).

The yield of glucose and xylose released was defined as the amount of solubilized monosaccharide divided by the total sugar content of the biomass prior to pretreatment as determined by chemical composition analysis. Prior to each analysis, standard curves were generated using pure solutions of glucose and xylose to convert peak area to concentration of monomeric sugar. The error bars in the figures represent the standard deviation from three or more biological replicates.

Chelating Inorganic Ions from Native Biomass

Biomass (3 g) was mixed with 30 mL of 0.2% (w/v) of the chelator diethylenetriaminepentaacetic acid (DTPA). The pH of the slurry was adjusted to 7.0 with 5 M NaOH, and the solution was incubated for 24 hr at 30° C. The biomass was then washed thoroughly with 10 volumes of distilled H₂O to remove the DTPA, dried at room temperature for 2 days, and stored in airtight bags. Biomass incubated for 24 hr at 30° C. with only deionized water was used as a control.

Pretreatment reactions as described above were performed either in the presence of 2,2′-bipyridine (bpy) plus Cu²⁺, Mn²⁺, or Fe²⁺ ([metal]:[bpy]=2 mM:4 mM) or in the absence of added bpy and metal to ascertain the effect these metal complexes had on the pretreatment of chelated woody biomass. The error bars represent the standard deviation from three or more biological replicates.

### Results and Discussion

Different woods are known to respond differently to oxidative pretreatment. To ascertain the key factors that lead to these variations, we analyzed the enzymatic digestibility of four hardwood samples (hybrid poplar, silver birch, aspen, and sugar maple) following alkali-only, AOP, or Cu-AOP pretreatment. Each of these biomass samples had differences in glucan and xylan composition, lignin content and composition, and redox active metal content, and these characteristics were compared with their enzymatic digestibilities following pretreatment.

### Cell Wall Composition Analysis

Cell wall composition is an important determinant in the enzymatic digestibility of lignocellulosic biomass, and not surprisingly, analysis of the biomass composition of the four different hardwood samples showed a range of lignin, hemicellulose and cellulose content (See Table 5 below). The amount of glucan varied from 32% to 44%, with the lowest content found in aspen (32%) and the highest in hybrid poplar (44%). Conversely, hybrid poplar had the lowest xylan content at 17%. Importantly, silver birch had the lowest lignin content (18%), while aspen, hybrid poplar, and sugar maple contained roughly equal amounts of lignin (25-26%).

### Comparison of Different Alkaline Pretreatments

To ascertain the susceptibility of silver birch, aspen, hybrid poplar, and sugar maple to different alkaline pretreatments, each biomass was subjected to alkali-only, AOP, or Cu-AOP pretreatment followed by enzymatic hydrolysis (FIG. 27 and Table 7). While silver birch and aspen both responded quite well to alkali-only pretreatment, the enzymatic digestibility of hybrid poplar and sugar maple rose much more modestly when only NaOH was used for pretreatment. Intriguingly, while three of the four woody biomass samples tested had very high levels of enzymatic digestibilities following AOP pretreatment and exhibited only a slight increase in digestibility when AOP was performed in the presence of copper 2,2′-bipyridine complexes (Cu-AOP), hybrid poplar behaved quite differently. In the case of hybrid poplar, enzymatic digestibility following alkali-only pretreatment and AOP pretreatment were nearly identical, while glucose yields more than doubled following Cu-AOP pretreatment. Xylose yields demonstrated very similar results (Table 7). Ultimately, silver birch exhibited the highest sugar yields following Cu-AOP and enzymatic hydrolysis (79%), followed by aspen (69%), hybrid poplar (60%), and sugar maple (51%).

TABLE 7
Glucose and xylose yields (%) following enzymatic hydrolysis
of alkaline, alkaline oxidative peroxide (AOP) and copper
catalyzed AOP (Cu-AOP) pretreatment for different biomasses

| | | | |
|---|---|---|---|
| Biomass | Alkaline | AOP | Cu-AOP |

Glucose yields (%)

| | | | |
|---|---|---|---|
| Silver Birch | 64.2 ± 3.4 | 74.0% ± 2.5 | 80.0 ± 1.1 |
| Aspen | 57.4 ± 5.0 | 63.3 ± 1.4 | 69.0 ± 2.3 |
| Hybrid Poplar | 32.5 ± 1.5 | 28.3 ± 0.2 | 60.6 ± 0.9 |
| Sugar Maple | 33.7 ± 0.3 | 48.7 ± 0.7 | 51.4 ± 0.8 |

Xylose hydrolysis yields (%)

| | | | |
|---|---|---|---|
| Silver Birch | 75.6 ± 3.9 | 78.12 ± 1.9 | 79.5 ± 1.3 |
| Aspen | 63.7 ± 2.5 | 61.35 ± 0.6 | 62.9 ± 2.1 |
| Hybrid Poplar | 59.5 ± 0.3 | 48.29 ± 0.01 | 74.6 ± 1.4 |
| Sugar Maple | 42.7 ± 0.1 | 56.43 ± 0.7 | 72.8 ± 0.4 |

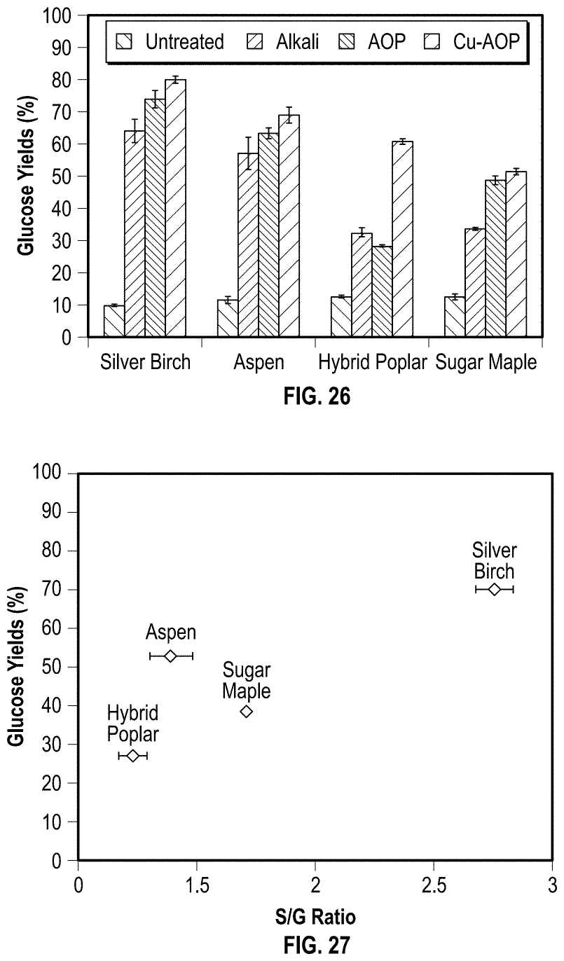

Silver birch, which had the lowest lignin content (Table 5), gave the highest sugar yields following enzymatic digestion (FIG. 26). However, the other three hardwoods all contained approximately the same amount of lignin yet differed significantly their enzymatic digestibility following pretreatment, indicating that there must be other differences between these four biomass samples. FIG. 26 shows glucose yields of various types of wood under various treatment conditions according to various embodiments.

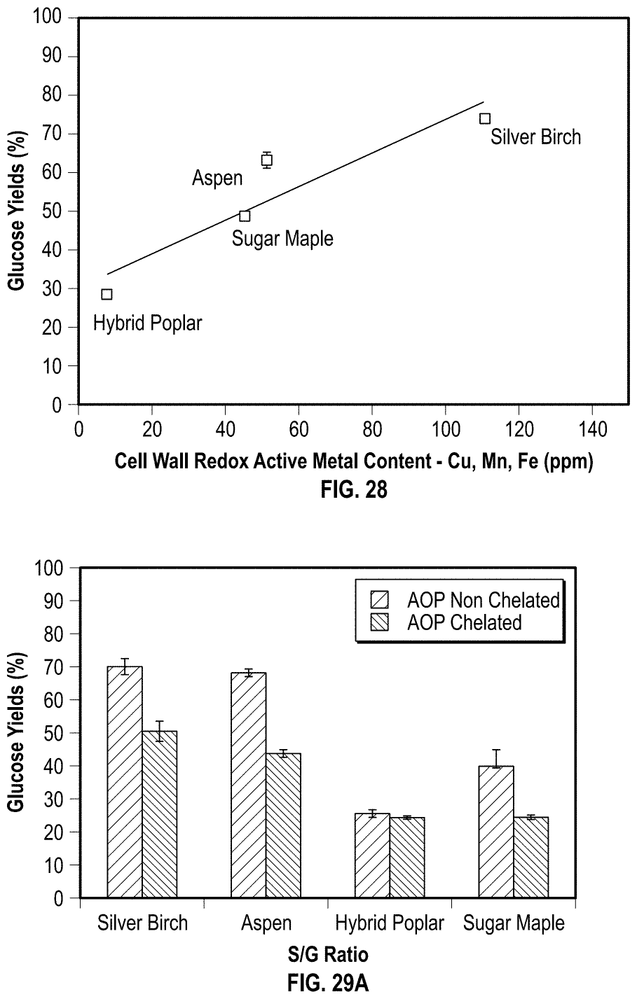

The S/G ratio, which is an important determinant in the extent of lignin crosslinking, is also known to affect biomass recalcitrance. S rich lignin has a less branched structure and lower degree of polymerization than G rich lignin, resulting in S rich lignin generally being more easily removed. In the present study, four different hardwoods were compared, and a correlation was found between the S/G ratio and biomass digestibility (FIG. 28). Silver birch, with the lowest lignin content and the highest S/G ratio (2.7), showed highest glucose yields with all pretreatments (Table 5, FIG. 30).

TABLE 5
Compositional analysis of hardwoods used in this study^{a}

| | | | Klason | |
|---|---|---|---|---|
| Biomass | Glucan (%) | Xylan (%) | Lignin (%) | S/G ratios |
| Silver birch | 39.0 ± 1.9 | 24.2 ± 0.1 | 17.8 ± 0.8 | 2.7 ± 0.1 |
| Aspen | 32.0 ± 1.7 | 20.1 ± 1.0 | 25.0 ± 0.9 | 1.3 ± 0.1 |
| Hybrid poplar | 44.0 ± 1.2 | 17.0 ± 0.2 | 24.6 ± 1.0 | 1.2 ± 0.1 |
| Sugar maple | 42.0 ± 2.0 | 20.0 ± 0.8 | 25.0 ± 2.5 | 1.7 ± 0.1 |

^{a}Errors represent standard deviations from either 6 (compositional analysis) or 3 biological replicates.

However, the digestibility of silver birch was only slightly higher than that of aspen even though aspen's S/G ratio was significantly lower (1.3). In addition, sugar maple had the second highest S/G ratio (1.7) and yet resulted in lower glucose yields following enzymatic digestion compared to both silver birch and aspen for all pretreatments tested. Together these results highlight the fact that while lignin content and composition are clearly important, other factors also impact how these four hardwoods respond to different alkaline pretreatments.

### Redox-Active Metal Content of the Cell Wall

It was noted that the addition of copper 2,2′-bipyridine complexes during AOP pretreatment (Cu-AOP) substantially increased the digestibility of hybrid poplar but only slightly increased the digestibility of the other hardwoods. It was hypothesized that AOP requires the presence of metal ions to be an effective pretreatment for hardwoods. It was further hypothesized that while the silver birch, aspen, and sugar maple samples already contained sufficient redox-active metal ions in their cell wall (thereby obviating the need for the additional copper ions during pretreatment), the hybrid poplar samples contained a relatively low level of redox-active metal ions. In this scenario, the addition of Cu(bpy) complexes during AOP pretreatment would compensate for the low natural levels of metal ions in our hybrid poplar samples.

To test these hypotheses, ICP-MS was performed to quantify cell wall redox-active metal ions in the four different hardwood samples (Table 6).

TABLE 6
ICP-MS analysis for total redox-active metals present in cell wall

| | Cell Wall Redox Metals (ppm) | | | |
|---|---|---|---|---|
| Biomass | Manganese | Iron | Copper | Total^{a} |
| Silver birch (control)^{b} | 100 | 10 | 1 | 111 ± 5 |
| Silver birch (non chelated)^{c} | 98 | 16 | 1 | 115 ± 3 |
| Silver birch (chelated)^{d} | 4 | 5 | 1 | 11 ± 2 |
| Aspen (control) | 19 | 26 | 6 | 51 ± 5 |
| Aspen (non chelated) | 16 | 23 | 6 | 45 ± 2 |
| Aspen (chelated) | 2 | 11 | 4 | 17 ± 3 |
| Hybrid poplar (control) | 1 | 5 | 1 | 7 ± 3 |
| Hybrid poplar (non | 1 | 5 | 1 | 7 ± 2 |
| chelated) | | | | |
| Hybrid poplar (chelated) | 1 | 2 | 1 | 4 ± 4 |
| Sugar maple (control) | 34 | 10 | 1 | 45 ± 3 |
| Sugar maple (non chelated) | 34 | 6 | 1 | 41 ± 4 |
| Sugar maple (chelated) | 3 | 2 | 1 | 6 ± 3 |

^{a}Errors represent the standard deviation from 3 biological replicates
^{b}Control samples are untreated biomass.
^{c}Non-chelated samples were incubated in pure deionized water for 24 hrs.
^{d}Chelated samples were treated with the chelator DTPA for 24 hrs as described in the experimental section.

The values ranged from only 7 ppm found in hybrid poplar to over 110 ppm in silver birch. As predicted, a strong positive correlation was discovered between the redox-active metal content of the woody biomass and enzymatic digestibility following AOP pretreatment (FIG. 28). This same correlation was not observed following Cu-AOP pretreatment. Analysis via ICP-MS of the cell wall metal ion content in Cu-AOP pretreated biomass demonstrated that all samples exhibited a very large increase in the amount of copper relative to the untreated samples, with copper essentially dominating the metal ratio (Table 8).

To corroborate the relationship between intracellular metal content and efficacy of AOP pretreatment, each of the different hard woods was incubated with the metal chelator DTPA prior to pretreatment. CP-MS analysis of the chelated biomass revealed a substantial decrease in metals, with DTPA treatment removing approximately 96% of cell wall redox metals (Table 6).

TABLE 8
ICP-MS concentrations (ppm) for untreated and
Cu-AHP, Fe-AHP and Mn-AHP pretreated biomasses

| | Cu-AHP | Fe-AHP | Mn-AHP |
|---|---|---|---|
| Biomass | (Copper) ppm | (Iron) ppm | (Manganese) ppm |
| Silver Birch | 1481 ± 6 | | |
| Aspen | 1238 ± 9 | | |
| Hybrid Poplar | 1182 ± 8 | 619 | 249 |
| Sugar Maple | 1176 ± 9 | | |

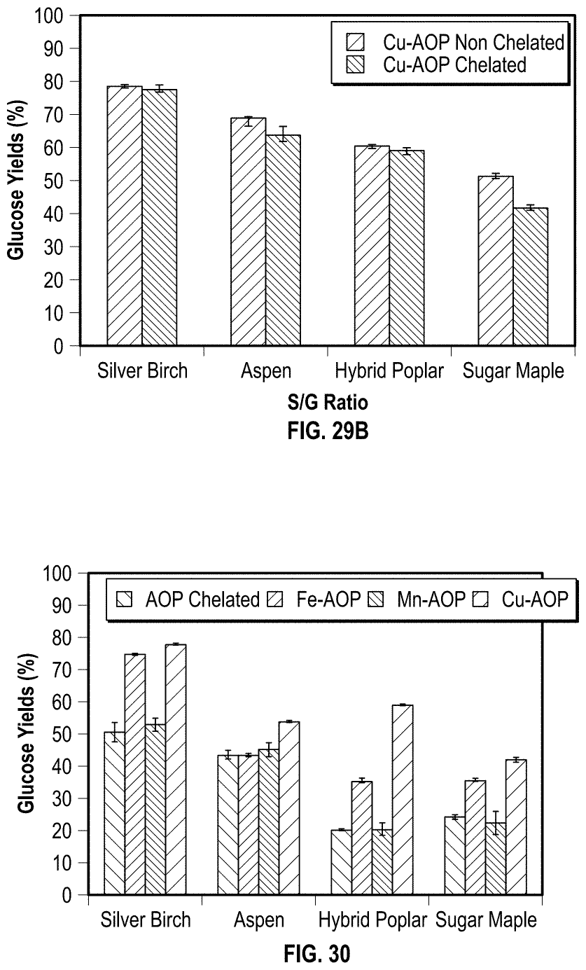

The effect of chelation was dramatic for biomass that initially contained a large amount of redox-active metal ions. For example, the enzymatic digestibility of chelated silver birch was significantly diminished following uncatalyzed AOP pretreatment relative to biomass that had not been chelated, with glucose yields reduced from 70% to only 50% (FIG. 29A). Likewise, chelated aspen and sugar maple also exhibited lower enzymatic digestibility following uncatalyzed AOP pretreatment relative to unchelated samples. Conversely, the digestibility of hybrid poplar, which naturally had very low cell wall metal content, was not affected by incubation with DTPA.

To verify that the decreased efficacy of uncatalyzed AOP following incubation with DTPA was due to the loss of the metal ions, chelated biomass was subjected to metal-catalyzed AOP pretreatment FIG. 29B. As expected, chelation of the hardwoods with DTPA prior to pretreatment did not significantly alter the efficacy of Cu-AOP, presumably because the addition of Cu(bpy) complexes obviated the need for naturally occurring intracellular metal ions. Interestingly, the addition of Cu(bpy) complexes led to significantly higher enzymatic digestibility than the addition of either Mn(bpy) or Fe(bpy) complexes (i.e. Mn-AOP and Fe-AOP) (FIG. 30). Whether this difference is due to the superior reactivity of Cu(bpy) complexes, the result of better penetration of Cu(bpy) complexes into the plant cell wall (Table 8), or some other property cannot be determined from this data.

### Example 14

### Two Oxidant Process

### Biomass

Materials—Hybrid poplar (Populus nigra var. charkoviensisxcaudina cv. NE-19) was collected from the University of Wisconsin Arlington Agricultural Research Station in 2012. The air-dried wood logs were chipped and hammer-milled (HammerHead, Muson Co., Inc. USA) to pass through a 5-mm screen. The milled biomass was stored in airtight bags prior to use. The chemical composition analysis of the biomass was 45.5% glucan, 15.8% xylan (sum of xylan, galactan, and manan), 22.3% Klason lignin, and 0.85% ash. The enzyme cocktails Cellic CTec3 and HTec3 were kindly provided by Novozymes A/S (Bagsværd, Denmark). Commercial Kraft hardwood lignin was provided by Suzano Papel e Celulose (São Paulo, Brazil). All other chemicals were reagent grade and purchased from Fisher Scientific (USA) unless otherwise noted.

### Pretreatment

Alkaline pre-extraction of biomass was performed at 10% NaOH loading on biomass (w/w based on the dry weight of the biomass) at 10% (w/v) solid consistency at either 90° C. or 120° C. for 1 hr. For the alkaline pre-extraction at 90° C., 100 g (dry basis) of hybrid poplar, 950 mL deionized water, and 50 mL 5 M NaOH were mixed and placed into a 2-L conical flask. Subsequently, the reactor was immersed into a water bath preheated to the target temperature. Every 10 min, the reaction mixture was mixed for 30 seconds by hand. The experiments for alkaline pre-extraction of poplar at 120° C. were performed using a 100-mL stainless steel Parr reactor (Parr Instruments Company, Moline, Ill., USA). For each experiment, 5 g (dry basis) of hybrid poplar, 47.2 mL of deionized water, and 2.5 mL of 5 M NaOH were incubated at 200 rpm for 1 h. After alkaline pre-extraction, the remaining insoluble fraction was separated by filtration and thoroughly washed with deionized water (about 1 L). The washed biomass was stored at 4° C. for subsequent analysis and use. The chemical composition of the biomass both before and after pre-extraction under these two temperatures is summarized in Table 9.

TABLE 9
Chemical composition of poplar after alkaline pre-extraction

| Substrate | Original | 90° C. | 120° C. |
|---|---|---|---|
| Solid remaining (%)^{a} | N/A | 87.1 ± 0.1 | 78.5 ± 0.1 |
| Glucan | 45.5 | 47.9 ± 0.8 | 55.0 ± 1.2 |
| Xylan | 15.8 | 16.4 ± 0.2 | 14.9 ± 0.2 |
| Klason lignin | 22.3 | 23.3 ± 0.3 | 21.1 ± 0.4 |
| Ash | 0.85 | 0.87 ± 0.1 | 0.86 ± 0.1 |

N/A: Not applicable.
Values are expressed as an average ± standard deviation of triplicate experiments.
^{a}Weight percentage (oven-dried weight) of the recovered biomass after alkaline pre-extraction relative to original biomass.

The second stage O₂-enhanced Cu-AHP (AHP/O) pretreatment was conducted using the same Parr reactor described above (for the pre-extraction at 120° C.) at a biomass loading of 10% (w/v) (based on the weight of original biomass). The oxidative pretreatment was performed with several fixed conditions, including 10% NaOH (based on the weight of original biomass), 1 mM CuSO₄, and 1 mM 2,2′-bipyridine (bpy). The bpy ligand is important to improve the delignification performance of the oxidative pretreatment. The underlying mechanism is unknown, but without being bound by theory it is thought that the aromatic N-donor ligand could improve the penetration of the copper ions into the cell wall to help localize the hydroxyl radicals formed from H₂O₂ and O₂. The reactions were performed at 80° C. while mixing with a stirrer at 200 rpm for 12 hr. The H₂O₂ loading was varied from 0-8% (w/w) (H₂O₂/original biomass). When O₂ was used as a co-oxidant, the O₂ pressure was fixed at 50 psi. After the oxidative pretreatment stage, the reactor was cooled in an ice/water bath and depressurized at room temperature. The solid fraction was separated from the liquor via filtration, thoroughly washed with deionized water (approximately 1 L), and stored at 4° C. prior to compositional analysis and enzymatic hydrolysis.

To recover the lignin solubilized during the alkaline-oxidative pretreatment process, the separated liquid phase was acidified to pH 2.0 with 72% (w/w) H₂SO₄. The precipitate was recovered by centrifugation (10 min at 11269×g), washed 2 times with aqueous H₂SO₄ (pH 2.0), and finally washed one time with cold deionized water. After centrifugation, the precipitated (lignin) was frozen at −80° C., lyophilized, and stored in the dark at 4° C.

### Enzymatic Hydrolysis

Enzymatic hydrolysis was performed in 15-mL pre-autoclaved (121° C. for 15 min) Falcon tubes using an enzyme cocktail consisting of CTec3 and HTec3 at a protein ratio of 1:1 and a total enzyme loading of 15 mg protein/g glucan (based on initial glucan content). Reactions were performed at 5% (w/v) solid loading (based on the weight of original, untreated biomass) and 50° C. for 72 h in 50 mM sodium citrate buffer (pH 5) with orbital shaking at 80 rpm (C24KC Incubator Shaker, New Brunswick Scientific, NJ, USA). Following enzymatic hydrolysis, the liquid phase was separated from the reaction mixture through centrifugation (10 min at 3075×g). The concentration of glucose and xylose released into solution was measured using a high-performance liquid chromatography (HPLC) system (Agilent 1260 Series) following a National Renewable Energy Laboratory (NREL) protocol. The yield of sugars was expressed on the basis of the initial sugar content of the original biomass prior to pretreatment (dry weight basis).

### Chemical Composition Analysis

The moisture content of the biomass was determined by drying at 105±2° C. to a constant weight. Before and after the pretreatment, the chemical composition of biomass was measured following an NREL two-stage hydrolysis protocol (See Sluiter A. et al., Determination of Structural Carbohydrates and Lignin in Biomass. National Renewable Energy Laboratory, NREL/TP-510-42618, Golden, Colo., 2012). In brief, the air-dried biomass was ground with a Wiley Mill to pass through 20 mesh screen. A sample (0.1 g) of the ground material was digested by the two-step H₂SO₄ hydrolysis protocol. After hydrolysis, the acid-insoluble lignin (Klason lignin) was separated by filtration, dried at 105±2° C., and weighed. The content of carbohydrates was quantified by an HPLC system (Agilent 1260 Series equipped with an infinity refractive index detector) fitted with a Bio-Rad Aminex HPX-87H column (Bio-Rad Laboratories, USA) using 5.0 mM sulfuric acid as the mobile phase with a flow rate of 0.6 mL min⁻¹ and an operation temperature of 65° C. The xylose content reported is actually the combination of xylose, mannose, and galactose because the HPX-87H column cannot separate these three sugars. Sugar quantification was accomplished by comparing the peak area to a standard curve prepared using pure glucose and xylose. The ash content of solid samples was determined by combustion of approximately 0.5 g of 20-mesh milled dry biomass at 550° C. for 12 hr.

### Lignin Characterization

The molecular weight distributions of lignin were analyzed using an Agilent 1260 Infinity HPLC equipped with a G1315D 1260 diode array detector VL, monitoring at 280 nm and recording 190-400 nm, a G1362A refractive index detector, and a Waters Ultrahydrogel 250 7.8×300 mm gel permeation chromatography (GPC) column attached to a Waters Ultrahydrogel 6×40 mm guard column. The temperature was held constant at 40° C. with 0.7 mL/min isocratic flow of 5 mM NaOH in 80/20 0.1 M aqueous sodium nitrate/acetonitrile as the mobile phase. Extracted lignin (20 mg dry weight) was dissolved in 1 mL eluent, and 25 μL of the sample was injected onto the HPLC. The retention times were compared to a sodium polystyrene sulfonate kit from Scientific Polymer Products (Mn 1,440-85,600 g/mol). The molecular weight at the highest peak (Mp) was calculated directly from external standards while the number average molecular weight (Mn), weight average molecular weight (Mw), and polydispersity (PDI) were calculated as described in Malawar, E. G. et al. Introduction to size exclusion chromatography. In: Wu C, editor. Handbook of size exclusion chromatography and related techniques (2nd ed.), New York, Marcel Dekker, 2004.

The hydroxyl content of the lignin samples was measured using phosphorus nuclear magnetic resonance (³¹P NMR) spectroscopy. Approximately 40 mg of the dry lignin was dissolved in 325 μL of a mixture of anhydrous pyridine and CDCl₃ (volume ratio of 1.6:1) and 300 μL anhydrous dimethylformamide (DMF). After completely dissolving the lignin, 100 μL cyclohexanol (stock concentration of 22 mg/mL) in anhydrous pyridine and CDCl₃ (volume ratio of 1.6:1) was added into the solution as an internal standard. Chromium (III) acetylacetonate (50 μL of a 5.6 mg/mL stock solution) in anhydrous pyridine and CDCl₃ (volume ratio of 1.6:1) was added into the mixture as a relaxation reagent. Phosphorylation of the lignin hydroxyl groups was initiated by the addition of 100 μL of the phosphitylation reagent 2-chloro-4,4,5,5-tetramethyl-1,3,2-dioxaphospholane. Analysis of the solution on an Agilent DDR2 500 MHz NMR spectrometer (relaxation delay of 5 s and 128 scans) equipped with a 7600AS autosampler and running VnmrJ 3.2A software. After obtaining the ³¹P NMR spectra, the hydroxyl content of the lignin sample was calculated based on the peak area ratio of the cyclohexanol internal standard (145.3.1-144.9 ppm) peak area to samples peaks, including aliphatic hydroxyl (149.1-145.4 ppm), condensed phenolic units (144.6-143.3; and 142.0-141.2 ppm), syringyl phenolic units (143.3-142.0 ppm), guaiacyl phenolic hydroxyl (140.5-138.6 ppm), p-hydroxyphenyl phenolic units (138.5-137.3 ppm), and carboxylic acid (135.9-134.0 ppm).

To evaluate the appropriateness of using the lignin in polyurethane formulations, the reactivity of the lignin towards isocyanate was tested. The reactivity of the lignin was determined using a titration method following a modified version of the American Society for Testing and Materials standard ASTM-D5155-14. Briefly, 1.0 g of methylene diphenyl diisocyanate (MDI) and 1.0 g of oven-dried lignin were dissolved in 6.0 g of DMF (dried with 4A molecular sieves, Fisher Scientific) and heated at 50° C. for 60 min. Then, 0.5 g of the solution was added to 25 mL of a dibutylamine solution (2 M) in toluene and mixed at 23° C. for 15 min at 150 rpm, followed by the addition of 110 mL of 2-propanol. After mixing, the solution was titrated with 1 M HCl to pH 4.2. A separate experiment was performed without the addition of the lignin-isocyanate sample as the control. Polyethylene glycol 400 (PEG 400) was used as a reference petroleum-based polyol. The amount of free isocyanate (unreacted isocyanate) was calculated according to equation (1):

$$\% NCO = \frac{4.2 \; 02 ( {V}_{1} - {V}_{2} ) \times M}{m} \qquad ( 1 )$$

where % NCO is the fraction of free (unreacted) isocyanate, V₁ is the volume of HCl required to reach pH 4.2 for the control sample (mL), V₂ is the volume of HCl required to reach pH 4.2 for lignin-isocyanate sample (mL), M is the molarity of HCl, and m is the weight (g) of the lignin/isocyanate sample added to the analysis mixture.

### Depolymerization of Lignin

Lignin oxidation was conducted according to modified literature procedures. See Rahimi A. et al., J. Am. Chem. Soc., 2013, 135, 6415-6418 and Nguyen, J. D. et al., J. Am. Chem. Soc., 2014, 136, 1218-1221. Briefly, 35 mg of lignin, 80 mg of Bobbitt's salt (4-acetamido-2,2,6,6-tetramethyl-1-oxopiperidinium tetrafluoroborate), 5 mL of 80:20 (v/v) acetonitrile:water, and 0.5 mL of 0.5 M HCl were placed into a 20-mL pressure tube. The reaction tube was sealed with a Teflon-lined cap and placed into a heating block at 80° C. for 2 h with magnetic stirring at 500 rpm. After cooling to room temperature, the solution was transferred into a 6-dram vial and concentrated under high vacuum (0.06 MPa) at 40° C. in a rotational vacuum concentrator (Thermo Scientific, SpeedVac Concentrator) to collect the oxidized lignin. The oxidized lignin was then subjected to formic acid-mediated lignin depolymerization to generate aromatic monomers as previously described in Rahimi, A. et al, Nature, 2014, 515, 249-252. Specifically, the dried lignin residue was dissolved in 5 mL of formic acid with sonication, and the solution was transferred into a 20 mL pressure tube. Sodium formate (40 mg) was added to the solution, and the tube was sealed and heated at 110° C. with magnetic stirring for 24 hr. The pressure tube was then cooled to room temperature, the solution transferred to a round-bottom flask, and the solvent removed using a rotary evaporator (Buchi Rotavapor). The lignin residue was suspended in water and the monomers were extracted 3 times with ethyl acetate. The organic fractions were combined and dried via rotary evaporation. The residue was dissolved in methanol and analyzed using a Prominence HPLC system (Shimadzu Scientific Instruments, USA) equipped with a RestekUltra C18 column (150×4.6 mm ID-3 μm particle size) and an SPD-M20A diode array detector. The system was maintained at 35° C. and the mobile phase was an CH₃CN:H₂O mixture containing 0.1% (v/v) formic acid. Monomers were detected and quantified by measuring the optical absorbance at 280 nm and 310 nm using 1,4-dimethoxybenzene (10 mM) as the internal standard loading control.

### Technoeconomic Analysis

TEA was performed using an Excel-based model derived from the NREL model for catalytic upgrading of sugars to hydrocarbons as described previously. See Davis, R. et al., Process Design and Economics for the Conversion of Lignocellulosic Biomass to Hydrocarbons: Dilute-Acid and Enzymatic Deconstruction of Biomass to Sugars and Catalytic Conversion of Sugars to Hydrocarbons. National Renewable Energy Laboratory, Harris Group Inc., NREL/TP-5100-62498, Golden, Colo., 2015. In brief, our model replaces the biomass and pretreatment modules of the NREL model with poplar and the two-stage alkaline pre-extraction followed by Cu-AHP pretreatment described here. A material balance was used to modify the sizing of all downstream operations, and this resizing was used to determine all capital, material, and energy costs.

The O₂ was assumed to be generated from air, with cost data derived from the National Energy Technology Laboratory and scaled appropriately. When O₂ was used as a co-oxidant, the pretreatment reaction vessels were rated for 0.7 MPa to account for the O₂ pressure and elevated temperature. Vessel thickness was adjusted based on design equations from Peters et al., and the cost of the vessel adjusted accordingly. The alkaline pre-extraction vessels were assumed to be rated for 0.3 MPa if performed at 90° C. and 0.5 MPa if performed at 120° C.

In addition to the pretreatment module, an evaporation module was included in the model to concentrate the carbohydrates solubilized during the pre-extraction and pretreatment. This enabled enzymatic hydrolysis to be performed at increased solid loadings. Carbohydrates solubilized from both the alkaline pre-extraction step and the second stage alkaline oxidative Cu-AHP process were assumed to be utilized for enzymatic hydrolysis.

The system boundary of this model does not include upgrading of lignin to final products. Instead, the solubilized lignin was assumed to be sold as-is at $0.80 per kg, approximately half the market value of polyols used for polyurethane. Currently, only acid precipitation has been tested as a means of isolating the lignin from both stages of the pretreatment process, which does not recover all of the lignin. Thus, two scenarios were tested: one in which only the lignin recovered via acid precipitation is sold (with the remainder burned for energy), and one in which all solubilized lignin is assumed to be recoverable.

Once all capital and operating costs were obtained, the minimum fuel selling price (MFSP) was obtained by fixing the 30-year net present value of the biorefinery to $0 using a 10% internal rate of return. All assumptions for calculating this rate of return are the same as in the NREL model. See Davis, R. et al., Process Design and Economics for the Conversion of Lignocellulosic Biomass to Hydrocarbons: Dilute-Acid and Enzymatic Deconstruction of Biomass to Sugars and Catalytic Conversion of Sugars to Hydrocarbons. National Renewable Energy Laboratory, Harris Group Inc., NREL/TP-5100-62498, Golden, Colo., 2015. Further details on cost assumptions are listed in the supplemental information (Table 10).

TABLE 10
Cost assumptions used in the economic model

| Item | Assumption | |
|---|---|---|
| Cost of poplar | $50/dry metric ton | |
| Cost of enzymes | $5/kg protein | |
| Cost of sodium hydroxide | $149/metric ton | |
| Cost of copper sulfate | $1.50/kg | |
| Cost of bipyridine | $30/kg | |
| Cost of hydrogen peroxide | $1.00/kg | |
| Lignin selling price | $0.80/kg | |
| Biorefinery throughput | 83,333 | kg/hour |
| Biorefinery operation | 8400 | hour/year |
| Pre-extraction reactor (90° C.) | $2,133,000 | installed cost |
| Pre-extraction reactor (120° C.) | $2,879,000 | installed cost |
| Pretreatment reactor (no O₂) | $15,746,000 | installed cost |
| Pretreatment reactor (with O₂) | $28,157,000 | installed cost |

### Statistical Analysis

Data was analyzed by a one-way analysis of variance with Unscrambler v10.5 software (CAMO software, Japan) followed by Tukey's post-hoc test. Significant differences between treatment conditions were evaluated at p≤0.05.

### Results

### Effect of Alkaline Oxidative Pretreatment on Lignin and Xylan Solubilization

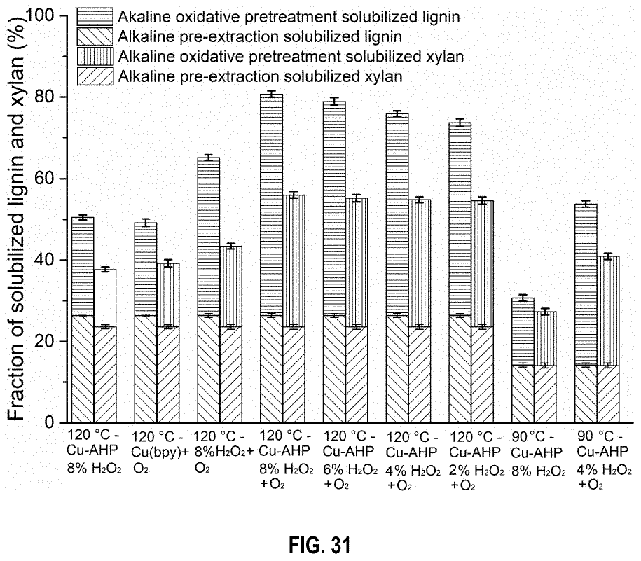

To understand the effect of O₂ as a co-oxidant during the alkaline-oxidative pretreatment on the biomass fractionation performance, a series of oxidative pretreatments with alkaline pre-extracted poplar was performed. After the oxidative pretreatment, the chemical composition of the remaining solid substrates was determined (Table 11). A substantial amount of lignin and xylan were solubilized during the oxygen-enhanced Cu-AHP (Cu-AHP/O) process, resulting in increased glucan percentage in the remaining solid (Table 11). FIG. 31 shows the solubilized lignin and xylan obtained following the two-stage alkaline pre-extraction/alkaline-oxidative pretreatment process. In FIG. 31, all reactions were performed for 13 hr at 10% (w/v) solids. The pre-extracted lignin (26.4% for 120° C. pre-extraction and 14.2% for 90° C. pre-extraction) and xylan (23.5% for 120° C. pre-extraction and 14.0% for 90° C. pre-extraction) during the alkaline pre-extraction were also included in the figure. The data are the average of triplicate experiments, and error bars represent ±standard deviation of the means. 120° C. and 90° C.: alkaline pre-extraction step conducted at 120° C. and 90° C., respectively. Cu-AHP H₂O₂: Cu-AHP pretreatment; Cu(bpy)+O₂: Cu(bpy)-catalyzed alkaline-oxidative pretreatment with O₂ as the only oxidant; Cu-AHP H₂O₂+O₂: O₂-enhanced Cu-AHP (Cu-AHP/O) pretreatment.

In the first set of experiments, shown on the far left of FIG. 31, three control oxidative pretreatments were performed, including Cu-AHP with H₂O₂ alone, copper 2,2′-bipyridine [Cu(bpy)]-catalyzed pretreatment with O₂ alone, and O₂-enhanced AHP [i.e., no Cu(bpy)]. Compared to the catalyzed pretreatment with H₂O₂ or O₂ alone, the utilization of both O₂ and H₂O₂ as co-oxidants removed more lignin and xylan even without the presence of Cu(bpy) as a catalyst. While the specific mechanism that led to this improvement is unknown, it has been hypothesized that H₂O₂ in the absence of Cu(bpy) may improve O₂ delignification in the pulp and paper industry by increasing the formation of hydroxyl radicals.

In the second series of experiments, shown in the middle of FIG. 31, we performed O₂-enhanced Cu-AHP (Cu-AHP/O) pretreatment with various H₂O₂ loadings [2, 4, 6, and 8% (w/w)]. The solubilized lignin and xylan during Cu-AHP/O pretreatment was significantly higher than those from the control reactions. As the H₂O₂ loading decreased from 8% (w/w) to 2% (w/w), the removal of lignin and xylan dropped slightly from approximately 55% and 33% to 48% and 32%, respectively, providing a reduction in the chemical inputs without sacrificing performance. In fact, the removed lignin and xylan during Cu-AHP/O pretreatment using only 2% (w/w) H₂O₂ loading was still much higher than the oxidative pretreatment with either 8% H₂O₂ only or 50 psi O₂ only. These results indicate that O₂ and H₂O₂ exhibit a synergistic effect in improving delignification and pretreatment efficacy.

In the third set of experiments, the impact of alkaline pre-extraction temperature on delignification performance of the oxidative pretreatment were examined, shown on the far right of FIG. 31. The reduction of alkaline pre-extraction temperature reduced the solubilization of lignin and xylan during the Cu-AHP alkaline-oxidative process. This is not surprising that pre-extraction at higher temperature removed more lignin and xylan from biomass, resulting in higher accessibility of residual biomass components to the chemicals utilized during the oxidative pretreatment stage. Moreover, the results obtained from biomass pre-extracted at 90° C. confirm the synergistic effect of using both O₂ and H₂O₂ as co-oxidants during alkaline oxidative processes, even at low H₂O₂ loadings. Following the two-stage alkaline pre-extraction/O₂-enhanced Cu-AHP (Cu-AHP/O) pretreatment, up to 75% of lignin and 55% of xylan were removed, which can be expected to significantly increase the enzymatic digestibility of the pretreated biomass.

TABLE 11
Chemical composition of poplar following the two-stage alkaline-oxidative
pretreatment process performed under various conditions.

| | | | | | Solid | | | Acid-insoluble |
|---|---|---|---|---|---|---|---|---|
| | O₂ | H₂O₂ | CuSO₄ | Bipyridine | remaining | Glucan | Xylan | lignin |
| Experiment # | (psi) | (%) | (mM) | (mM) | (%)^{a} | (%)^{b} | (%)^{b} | (%)^{b} |
| Untreated | N/A | N/A | N/A | N/A | N/A | 45.5 | 15.8 | 22.3 |
| 120° C. - Cu-AHP | — | 8 | 1 | 1 | 68.6 ± 0.7 | 58.1 ± 0.6 | 14.2 ± 0.5 | 16.1 ± 0.5 |
| 8% H₂O₂ | | | | | | | | |
| 120° C. - Cu(bpy) | 50 | — | 1 | 1 | 69.6 ± 0.7 | 56.8 ± 0.8 | 13.6 ± 0.3 | 16.3 ± 0.3 |
| + O₂ | | | | | | | | |
| 120° C. - AHP 8% | 50 | 8 | — | — | 65.8 ± 1.0 | 61.0 ± 0.8 | 14.1 ± 0.4 | 11.8 ± 0.2 |
| H₂O₂ + O₂ | | | | | | | | |
| 120° C. - Cu-AHP | 50 | 8 | 1 | 1 | 52.2 ± 0.6 | 74.7 ± 1.1 | 12.9 ± 0.6 | 8.2 ± 0.5 |
| 8% H₂O₂ + O₂ | | | | | | | | |
| 120° C. - Cu-AHP | 50 | 6 | 1 | 1 | 52.6 ± 0.6 | 74.4 ± 0.9 | 13.1 ± 0.5 | 8.9 ± 0.4 |
| 6% H₂O₂ + O₂ | | | | | | | | |
| 120° C. - Cu-AHP | 50 | 4 | 1 | 1 | 53.2 ± 0.4 | 73.7 ± 1.2 | 13.0 ± 0.6 | 10.1 ± 0.5 |
| 4% H₂O₂ + O₂ | | | | | | | | |
| 120° C. - Cu-AHP | 50 | 2 | 1 | 1 | 54.1 ± 0.8 | 73.3 ± 1.3 | 12.9 ± 0.5 | 10.8 ± 0.4 |
| 2% H₂O₂ + O₂ | | | | | | | | |
| 90° C. - Cu-AHP | — | 8 | 1 | 1 | 76.3 ± 0.6 | 54.6 ± 0.9 | 15.1 ± 0.6 | 20.3 ± 0.6 |
| 8% H₂O₂ | | | | | | | | |
| 90° C. - Cu-AHP | 50 | 4 | 1 | 1 | 71.4 ± 0.8 | 57.9 ± 1.1 | 13.1 ± 0.3 | 14.7 ± 0.8 |
| 4% H₂O₂ + O₂ | | | | | | | | |

N/A: Not applicable.
120° C. and 90° C.: alkaline pre-extraction step conducted at 120° C. and 90° C., respectively.
Cu-AHP H₂O₂: Cu-AHP pretreatment; Cu(bpy) + O₂: Cu(bpy)-catalyzed alkaline-oxidative pretreatment with O₂ as the only oxidant; Cu-AHP H₂O₂ + O₂: (T-enhanced Cu-AHP (Cu-AHP/O) pretreatment.
Values are expressed as average ± standard deviation of triplicate experiments.
^{a}Weight percentage (oven-dried weight) of the recovered biomass after two-stage Cu-AHP pretreatment relative to original biomass.
^{b}Weight percentage based on oven-dried weight of two-stage pretreated biomass.

### Enzymatic Hydrolysis Yields

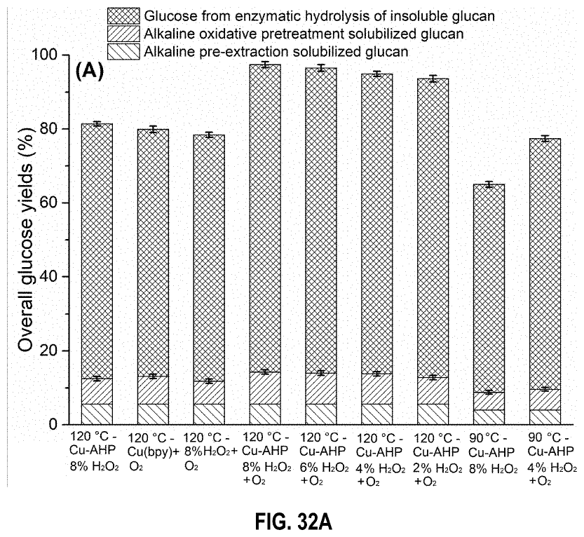

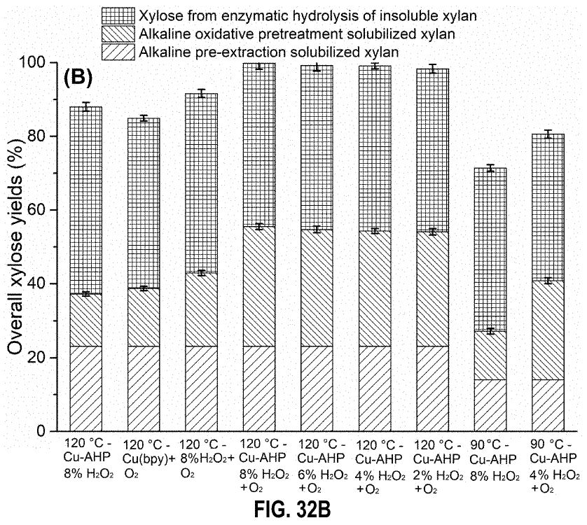

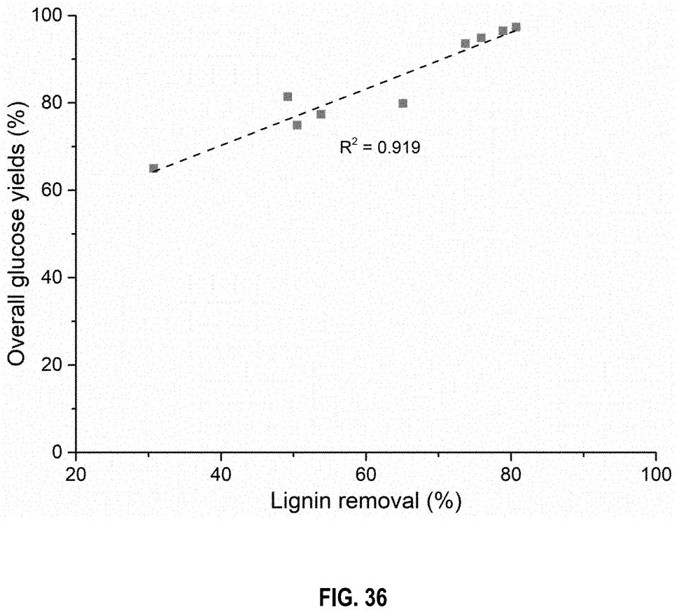

Enzymatic hydrolysis was performed on the pretreated biomass to determine the extent of recovered sugars following delignification. FIGS. 32A and 32B shows the overall sugar yields of glucose and xylose following the two-stage pretreatment (alkaline pre-extraction followed by alkaline-oxidative Cu-AHP pretreatment) and enzymatic hydrolysis of hybrid poplar. In FIGS. 32A and 32B, enzymatic hydrolysis was performed at 50° C. and pH 5 using 15 mg of total protein per g of glucan (based on initial glucan content) for 72 hr. The data are an average of triplicate experiments, and error bars represent ±standard deviation of the means. The pre-extracted glucan (5.6% for 120° C. pre-extraction and 4.0% for 90° C. pre-extraction) and xylan (23.5% for 120° C. pre-extraction and 14.0% for 90° C. pre-extraction) during alkaline pre-extraction stage are indicated in black. 120° C. and 90° C.: alkaline pre-extraction step conducted at 120° C. and 90° C., respectively. Cu-AHP H₂O₂: Cu-AHP pretreatment; Cu(bpy)+O₂: Cu(bpy)-catalyzed alkaline-oxidative pretreatment with O₂ as the only oxidant; Cu-AHP H₂O₂+O₂: O₂-enhanced Cu-AHP (Cu-AHP/O) pretreatment. Sugar yields were calculated based on the original glucan and xylan content. The carbohydrates solubilized during the alkaline pre-extraction stage (glucan=5.6% at 120° C. and 4.0% at 90° C.; xylan=23.5% at 120° C. and 14.0% at 90° C.) were also included because these solubilized sugars could theoretically be recovered. It is notable that the combination of O₂ and H₂O₂ as co-oxidants achieved much higher overall glucose yields compared to using H₂O₂ or O₂ individually (FIG. 32A). When conducting Cu-AHP/O pretreatment on the 120° C. alkaline pre-extracted poplar, the reduction of the H₂O₂ loading from 8% to 2% (w/w) only slightly decreased the overall glucose yields (from ˜97% to ˜93%). In contrast, when conducting oxidative pretreatments of the same 120° C. alkaline pre-extracted poplar using either 50 psi O₂ or 8% (w/w) H₂O₂ independently as oxidants, the overall glucose yields were in the range of 79-82% (based on initial glucan content). This not only indicates the efficacy of using both O₂ and H₂O₂ as co-oxidants in alkaline oxidative pretreatments, it also provides an opportunity to improve the processing economics by reducing the chemical (H₂O₂) inputs while simultaneously increasing sugar yields. Decreasing the alkaline pre-extraction temperature from 120 to 90° C. led to a predictable reduction in overall sugar yields (FIGS. 32A and 32B). A plot of overall glucose yields versus extent of lignin removal revealed a strong positive correlation (FIG. 36), further supporting that lignin removal during delignifying pretreatments can be a good indicator of glucose yields. Xylose yields displayed a similar trend, revealing a decrease in yield using the lower pre-extraction temperature as well as much higher yields for the O₂-enhanced Cu-AHP (Cu-AHP/O) pretreatment relative to Cu-AHP with H₂O₂ only (FIG. 32B). Taken together, these data highlight that O₂ is a promising co-oxidant in combination with H₂O₂ to enhance alkaline-oxidative pretreatment efficacy.

### Impact of Pretreatment on Recovered Lignin Properties

Valorization of the isolated lignin is critical to improve the process economics of an integrated biorefinery. One of the proposed value-added applications of lignin streams is in the formulation of polyurethanes because the hydroxyl groups in lignin can react with isocyanate to form urethane linkages. The use of lignin can reduce or even eliminate the need for petroleum-based polyols during the production of polyurethanes. For example, lignin was recently used to replace 100% of the phenol during the production of phenol-formaldehyde resin. Critical polyol parameters that impact polyurethane properties are the number, types (e.g., aliphatic versus phenolic), and accessibility of hydroxyl groups, the number of carboxylates, the solubility of the polyol, and its molecular weight distribution. In the case of lignin, these properties are a complex function of the type of biomass, the isolation method, and the conditions (pH and temperature) used to extract lignin.

Lignin isolated from both the Cu-AHP and O₂-enhanced Cu-AHP (Cu-AHP/O) processes was characterized in terms of molecular weight, hydroxyl group content, and reactivity to isocyanate. Cu-AHP pretreatment employing H₂O₂ as the sole oxidant results in higher average molecular weight (Mₙ) lignin than that of the pretreatments with O₂ alone or the combination of O₂ and H₂O₂ (Table 12). The combination of O₂ and H₂O₂ as co-oxidants only slightly altered the Mn compared to pretreatment with O₂ only. This suggests that O₂ plays a more important role than H₂O₂ in cleaving lignin chains into small pieces and/or reducing lignin condensation/crosslinking during the reaction. This conclusion is supported by the Mw values, which account for the fraction of large molecular weight chains in the polymer. Interestingly, varying the oxidative pretreatment conditions did not change the Mp significantly. Analysis of the lignin precipitated following Cu-AHP/O by heteronuclear single quantum coherence (HSQC) NMR indicated that this lignin is essentially identical to the previously characterized lignin obtained using the original two-stage Cu-AHP strategy (i.e., no O₂), although the β-O-4 content might be slightly reduced in lignin precipitated following Cu-AHP/O pretreatment (data not shown). In general, lignin isolated from the Cu-AHP/O pretreatment process is characterized to have short chains and low PDI, potentially favorable characteristics for the production of value-added products.

TABLE 12
Molecular weight distribution of the lignin samples from
different alkaline-oxidative pretreatment conditions

| | | | | Polydis- |
|---|---|---|---|---|
| | Mn | Mw | Mp | persity |
| Pretreatment conditions^{a} | (g/mol) | (g/mol) | (g/mol) | (PDI) |
| 120° C. - Cu-AHP 8% H₂O₂ | 1618 | 86870 | 2040 | 5.4 |
| 120° C. - Cu(bpy) + O₂ | 4950 | 50560 | 1500 | 10.2 |
| 120° C. - Cu-AHP 8% H₂O₂ + O₂ | 8280 | 47330 | 2200 | 5.7 |
| 120° C. - Cu-AHP 4% H₂O₂ + O₂ | 6940 | 42050 | 1830 | 6.1 |
| 90° C. - Cu-AHP 4% H₂O₂ + O₂ | 6690 | 57150 | 1940 | 8.5 |

^{a}120° C. and 90° C.: alkaline pre-extraction step conducted at 120° C. and 90° C., respectively. Cu-AHP H₂O₂: Cu-AHP pretreatment; Cu(bpy) + O₂: Cu(bpy)-catalyzed alkaline-oxidative pretreatment with O₂ as the only oxidant; Cu-AHP H₂O₂ + O₂: O₂-enhanced Cu-AHP (Cu-AHP/O) pretreatment.

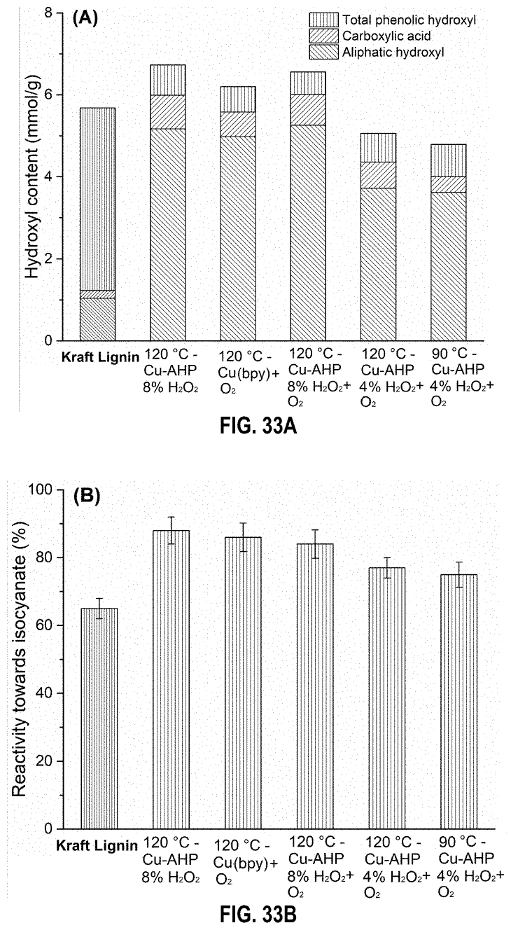

To assess the suitability of the lignin as a polyol replacement for the production of polyurethane, the hydroxyl content, the type of hydroxyl group, and the reactivity of the lignin towards isocyanate were determined. In FIGS. 33A and 33B are graphs showing the impact of Cu-AHP pretreatment conditions on (FIG. 33A) the hydroxyl group content and (FIG. 33B) the reactivity towards isocyanate (MDI) of the lignin samples. The data shown in FIG. 33B are the average of triplicate measurements of the same batch lignin, and error bars represent ±standard deviation of the means. 120° C. and 90° C.: alkaline pre-extraction step conducted at 120° C. and 90° C., respectively. Cu-AHP H₂O₂: Cu-AHP pretreatment; Cu(bpy)+O₂: Cu(bpy)-catalyzed alkaline-oxidative pretreatment with O₂ as the only oxidant; Cu-AHP H₂O₂+O₂: O₂-enhanced Cu-AHP (Cu-AHP/O) pretreatment.

As shown in FIG. 33A, compared to a commercial Kraft hardwood lignin which has aliphatic and phenolic hydroxyl groups of 1.04 and 4.45 mmol/g, respectively, lignin isolated from the Cu-AHP pretreatment process had significantly higher aliphatic hydroxyl content (3.62-5.26 mmol/g) and lower phenolic hydroxyl content (0.55-0.79 mmol/g). This is probably due to the significant depolymerization of Kraft hardwood lignin during the pulping process that yields a high number of phenolic hydroxyl content, while the metal-catalyzed alkaline oxidative pretreatment only results in minimal lignin condensation or depolymerization. The utilization of both O₂ and H₂O₂ as co-oxidants resulted in a slight reduction in the total hydroxyl content (FIG. 33A).

The content and type of hydroxyl group is strongly correlated with the reactivity of the lignin, and it was therefore not unexpected that Cu-AHP lignin generally demonstrated a much higher reactivity than Kraft hardwood lignin (FIG. 33B). This is presumably because Cu-AHP lignin has a significantly higher aliphatic hydroxyl content (FIG. 33A) and aliphatic hydroxyl groups are more reactive than aromatic hydroxyl groups towards isocyanate. Importantly, the reactivity of all the Cu-AHP lignins tested using a variety of oxidation conditions ranged from 75-85%, which are significantly higher than the reactivity of commercial Kraft hardwood lignin (65%), and closer to the reactivity of petroleum-based polyol (PEG 400) that we measured using similar method (90.5%). This indicates the lignin isolated through Cu-AHP process is a promising candidate to entirely replace petroleum-based polyol in polyurethane formulations.

### Depolymerization of Recovered Lignin

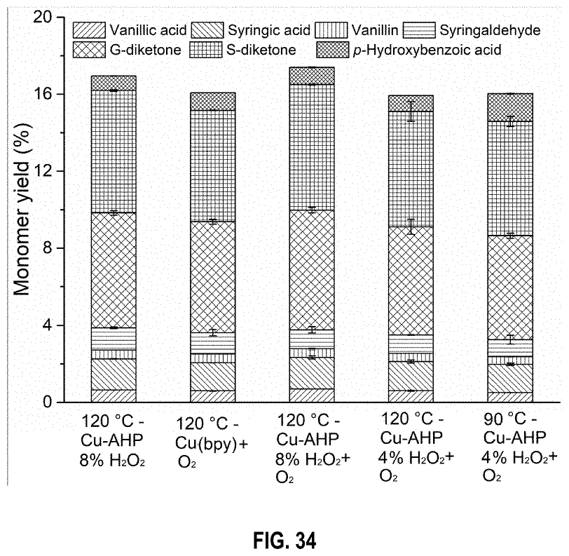

To further examine the impact of oxidative pretreatment on the lignin properties, the monomer yields of the lignin were determined using a sequential Bobbitt's salt-based oxidation and formic acid-mediated depolymerization process (FIG. 34). FIG. 34 is a graph showing monomer yields (on a lignin basis) from a sequential oxidation and depolymerization of lignin isolated using different alkaline-oxidative pretreatment conditions; 120° C. and 90° C.: alkaline pre-extraction step conducted at 120° C. and 90° C., respectively; Cu-AHP H₂O₂: Cu-AHP pretreatment; Cu(bpy)+O₂: Cu(bpy)-catalyzed alkaline-oxidative pretreatment with O₂ as the only oxidant; Cu-AHP H₂O₂+O₂: O₂-enhanced Cu-AHP (Cu-AHP/O) pretreatment. Following sequential oxidation and depolymerization, the S/G ratio of the depolymerized monomers from different Cu-AHP lignin samples was consistently in the range of 1.23-1.32, which is in a good agreement with previous results reported on poplar lignin. Moreover, these results indicate that Cu-AHP pretreatment employing O₂ and H₂O₂ as the oxidant(s) either individually or in combination does not selectively cleave or crosslink S or G units of the lignin, which is favorable for further lignin depolymerization into aromatic monomers. The dominant monomers generated through this process are diketones, attributed to the cleavage of the β-O-4 bond through an aryl vinyl ether intermediate that is formed in the formic acid catalyzed depolymerization step. Importantly, following the sequential oxidation and depolymerization process, the monomer yields of the Cu-AHP lignin samples were not appreciably different from one another, further highlighting that using both O₂ and H₂O₂ as co-oxidants does not negatively impact lignin quality or the overall depolymerization efficiency. Lignin samples prepared using different pre-extraction temperature (120° C. vs. 90° C.) also gave similar monomer yields, except that the lower pre-extraction temperature led to slightly higher yields of p-hydroxybenzoic acid, consistent with increased retention of the corresponding ester linkage at the lower pretreatment temperature. Thus, the higher alkaline pre-extraction temperature that resulted in substantially increased sugar yield did not simultaneously lead to a decrease in the quality of the residual lignin. Taken together, these results demonstrate that the use of O₂ as a co-oxidant in the Cu-AHP pretreatment process maintains the high quality of the lignin for efficient depolymerization and further valorization.

### Technoeconomic Analysis

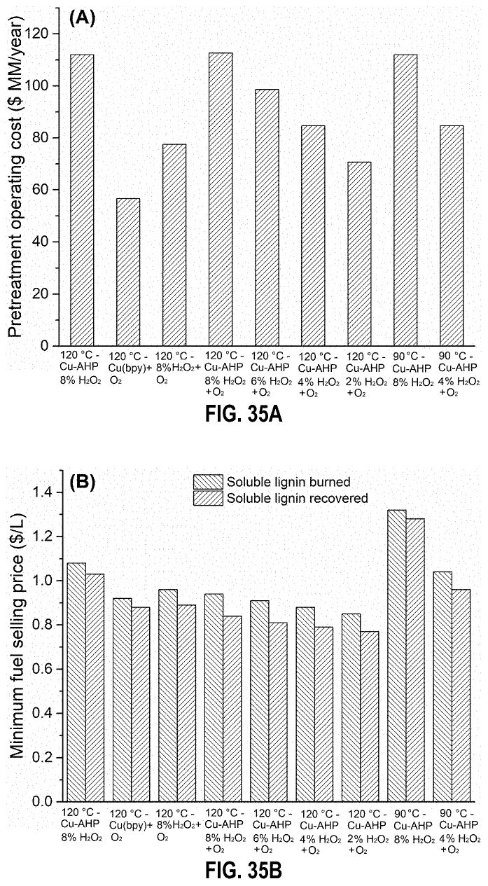

The minimum fuel selling price (MFSP) ($/L) and pretreatment operating costs (million $/year) for the nine pretreatment conditions are shown in FIGS. 35A and 35B. FIGS. 35A and 35B show graphs of pretreatment operating cost (FIG. 35A) in million $/year and minimum fuel selling price (MFSP) (FIG. 35B) in $/L for various Cu-AHP pretreatment conditions. MFSP is shown assuming non-precipitated soluble lignin in the extract of the second pretreatment stage is either burned for energy or recovered for high value lignin products. 120° C. and 90° C.: alkaline pre-extraction step conducted at 120° C. and 90° C., respectively. Cu-AHP H₂O₂: Cu-AHP pretreatment; Cu(bpy)+O₂: Cu(bpy)-catalyzed alkaline-oxidative pretreatment with only O₂ as the oxidant; Cu-AHP H₂O₂+O₂: O₂-enhanced Cu-AHP (Cu-AHP/O) pretreatment. Two scenarios are presented for the MFSP. In the first scenario, the soluble lignin that is not precipitated is burned for energy, while in the second scenario, the soluble lignin in the Cu-AHP extract that is not precipitated is assumed to be recoverable and sold at the same price as the precipitated lignin. The cost of pretreatment chemicals has a large influence on the MFSP, accounting for 40% of the total operating costs for the base case of a 120° C. alkaline pre-extraction followed by an alkaline oxidative Cu-AHP pretreatment with 8% H₂O₂ (120° C.—Cu-AHP 8% H₂O₂). Using O₂ in addition to H₂O₂ during the alkaline oxidative pretreatment only slightly increases operating costs [(120° C.—Cu-AHP 8% H₂O₂) versus (120° C.—Cu-AHP 8% H₂O₂+O₂)] due to the electricity usage required to produce and concentrate oxygen from air. In contrast, reducing H₂O₂ utilization from 8% to 2% reduces operating costs by $42 million/year due to the relatively high cost to purchase H₂O₂ (about $1.00/kg). Because this sizable reduction in pretreatment chemical usage did not result in a correspondingly large reduction in sugar yield (FIGS. 32A and 32B), this led to a considerable decrease in MFSP. If we assume that the acid-soluble lignin is not recoverable, the MFSP using H₂O₂ as the only oxidant [(120° C.—Cu-AHP 8% H₂O₂) and (90° C.—Cu-AHP 8% H₂O₂)]) is between $1.32/L and $1.08/L depending on the temperature of the alkaline pre-extraction stage. Conversely, when O₂ is used as a co-oxidant and the H₂O₂ loading is reduced from 8% to 2%, the MFSP decreased to between $0.94/L to $0.85/L. Eliminating the H₂O₂ entirely led to slight increase in MFSP due to an appreciable reduction in both the sugar and lignin yields.

Importantly, if the acid-soluble lignin can be recovered for value-added products, then the MFSP can be reduced by an additional $0.10/L (down to $0.77/L) if O₂ is employed as a co-oxidant during the Cu-AHP process (120° C.—Cu-AHP 2% H₂O₂+O₂). The use of O₂ as a co-oxidant increases the amount of lignin solubilized during pretreatment, but a larger proportion of this lignin is acid soluble. Thus, the difference in MFSP between the two assumptions (all solubilized lignin is recoverable versus only precipitated lignin) is greater when O₂ is employed as a co-oxidant.

### Discussion

In this study, an improved alkaline oxidative pretreatment using both O₂ and H₂O₂ as co-oxidants was identified that efficiently delignifies biomass for sugar production while also providing a lignin stream amenable to subsequent valorization. Following enzymatic hydrolysis of the O₂-enhanced Cu-AHP pretreated biomass, total net sugar yields of ˜93% for glucose and ˜100% for xylose (based on the initial composition) can be achieved using a relatively low H₂O₂ loading (2% w/w). Compared to earlier studies of the two-stage Cu-AHP pretreatment using H₂O₂ as the only oxidant, using a combination of O₂ and H₂O₂ as co-oxidants substantially improved pretreatment efficiency and enabled reduced chemical inputs. Moreover, integrating the published data on lignin removal and monomeric sugar yields during Cu-AHP pretreatment employing different H₂O₂ loadings [0, 2, 4, 6% (w/w)] (See Yuan et al. Ind Eng Chem Res 2019, 58:16000-16008) with the results obtained in this study (FIGS. 31, 32A, and 32B) indicates a synergistic effect of using both O₂ and H₂O₂ as co-oxidants during the Cu-catalyzed alkaline-oxidative pretreatment of lignocellulosic biomass. Although there is literature precedent for enhancing O₂-dependent delignification of pulp by the addition of H₂O₂ or CuSO₄, to our knowledge there are no published reports describing the simultaneous use of O₂ and H₂O₂ as co-oxidants during the oxidative pretreatment of lignocellulosic biomass to recover both lignin and monomeric C-5 and C-6 sugars.

In the proposed two-stage pretreatment process, following solubilization of ˜25% of the original lignin in an alkaline pre-extraction stage (conducted at 120° C.), the Cu-catalyzed alkaline-oxidative pretreatment using the combination of O₂ and H₂O₂ as co-oxidants removes another ˜50% of the lignin. The latter process stage remains effective even at a H₂O₂ loading as low as 2% (w/w, based on initial biomass). The removal of such high amount of lignin (˜75% of the initial lignin) results in the significant increase of the enzymatic digestibility of the biomass; this is part of the reason for high sugar yields following enzymatic hydrolysis. Moreover, the dissolved lignin in the liquid phase can theoretically be recovered at high purity and employed in a variety of lignin valorization strategies, including the production of polyurethanes or depolymerization into aromatic monomers.

The lignin isolated from Cu-AHP pretreatment with or without the addition of O₂ has higher hydroxyl group content (especially aliphatic hydroxyl group) and reactivity with isocyanate (MDI) relative to commercial Kraft hardwood lignin (FIG. 33), and it is similar to lignin obtained from other oxidative pretreatment processes, such as magnesium sulfate-catalyzed alkaline oxygen pretreatment of Eucalyptus. In addition, compared to lignin isolated from alkaline oxygen or alkaline hydrogen peroxide pretreatment of rice straw or wheat straw, lignin isolated during the O₂-enhanced Cu-AHP (Cu-AHP/O) pretreatment of poplar has much higher aliphatic hydroxyl content. These results suggest that O₂-enhanced Cu-catalyzed alkaline oxidative pretreatment can provide a lignin stream that may be very amenable to the formulation of polyurethanes.

The impact of delignification conditions on lignin properties was also evaluated through a sequential oxidation and formic acid depolymerization method. Oxidation of C_{α}—OH to C_{α}═O through Bobbitt's salt oxidation is an efficient approach to decrease β-O-4 ether bond dissociation energy, making is susceptible to subsequent formic acid-catalyzed depolymerization. Significantly, the monomer yield of lignin isolated from our two-stage Cu-AHP pretreatment is 2.4 and 1.5 times higher than that obtained from an organosolv-isolated lignin and enzymatic lignin, respectively (data not shown). The monomer yield variation of different Cu-AHP lignin samples was very minor, suggesting that the addition of O₂ as a co-oxidant did not have a significant impact on the lignin properties. Thus, the combination of O₂ and H₂O₂ as co-oxidants during alkaline oxidative pretreatment consistently produced high-quality lignin samples that can be effectively depolymerized for further valorization.

TEA indicates that the overall MFSP can be reduced nearly 40% by using O₂ as a co-oxidant in the Cu-AHP process relative to the Cu-AHP pretreatment using H₂O₂ only. This is due both to a decrease in pretreatment operating cost (due to a reduction in H₂O₂ loading) and to an increase in both glucose and lignin yield. The primary tradeoff of oxygen utilization is a modest increase in electricity usage to generate the oxygen as well as an increase in capital costs (the oxygenator is assumed to cost $9.7 million, while the cost of increasing the pressure rating of the pretreatment vessel is $12.4 million (Table 11). Despite these costs, the added capital cost only increased ˜6% and therefore did not greatly impact the MFSP.

Further reductions in the MFSP are feasible if improved utilization of solubilized lignin can be achieved. While more than 50% of the total lignin was solubilized during O₂-enhanced Cu-AHP, only approximately one-third of that solubilized lignin could be precipitated at pH 2. Further research on alternate methods to isolate this lignin is warranted, as utilizing this lignin could potentially reduce the MFSP to as low as $0.77/L (FIGS. 35A and 35B). Furthermore, although our TEA assumed a lignin selling price of only $0.80/kg, we have demonstrated that the lignin recovered from our O₂-enhanced Cu-AHP pretreatment can be depolymerized into potentially higher-value aromatic monomers. Because this technology is less developed, however, it was not considered in the TEA. Given the promising depolymerization results, however, the MFSP will be lowered even further as this technology matures. Thus, improved delignifying pretreatment methods employing both O₂ and H₂O₂ as co-oxidants in alkaline oxidative pretreatment processes can enable the economically viable conversion of biomass into biofuels and biochemicals.

### Conclusions

In this study, using both O₂ and H₂O₂ as co-oxidants under alkaline conditions was demonstrated as an effective biomass fractionation strategy which can simultaneously generate both highly digestible biomass for sugar production and a high-quality lignin stream amenable to subsequent valorization. In addition, employing O₂ as a co-oxidant enables a substantially reduced H₂O₂ loading to be used while still achieving high yields of monomeric sugar following enzymatic hydrolysis. The lignin isolated from the O₂-enhanced Cu-AHP (Cu-AHP/O) pretreatment process exhibits a high content of hydroxyl groups (especially aliphatic hydroxyls) and substantial reactivity to isocyanate, suggesting that the lignin might be very useful for the production of polyurethanes. In addition, the lignin was also susceptible to sequential oxidation and depolymerization, indicating that the lignin might also be useful for the production of aromatic monomers. Even when assuming a relatively low lignin value ($0.80/kg), TEA indicates that the synergistic combination of O₂ and H₂O₂ for the second Cu-AHP pretreatment stage could reduce the MSFP to as low as $0.77 per liter biofuels. These data demonstrate the potential of employing both O₂ and H₂O₂ as co-oxidants during alkaline oxidative processes.

In one embodiment, a commercial process for the production of lignocellulose derived sugars is provided. This process can, for example, be converted by biological, chemical, or catalytic conversion to renewable biofuels, biochemicals, and biopolymers. This may function as a standalone pretreatment, or as a component of a multi-stage pretreatment process (as in the case of oxidative bleaching or delignification following alkaline pulping).

The various embodiments further include a homogeneous catalyst system comprising one or more metals; at least one metal coordinating ligand; and at least two oxidants, wherein the homogeneous catalyst system is a metal-ligand complex configured to catalytically delignify alkaline pretreated lignocellulosic biomass with the at least two oxidants in an oxidative pretreatment reaction.

In one embodiment, the oxidants are selected from air, oxygen, hydrogen peroxide, persulfate, percarbonate, sodium peroxide, ozone and combinations thereof. In one embodiment, the oxidants are oxygen and hydrogen peroxide. In one embodiment, the oxygen pressure is from about 25 psi to about 100 psi and the hydrogen peroxide is less than about 5% (w/w).

In various embodiments, the one or more metals are metal ions capable of oxidation and/or reduction. In one embodiment, the metals ions are selected from Fe(II), Fe(III)), Cu(I), Cu(II), Co(III), Co(VI)), V(II), V(III), V(IV), V(V) and combinations thereof. In one embodiment, the metal-ligand complex is a copper-ligand complex. In one embodiment, the metal coordinating ligand is selected from pyridine, 1,10-phenanthroline, ethylenediamene, histidine, glycine and combinations thereof. In one embodiment, the metal-ligand complex is copper 2,2′-bipyridine (Cu(bpy)).

The various embodiments further include a method of pretreating plant biomass comprising catalytically pretreating the plant biomass with a metal-ligand complex and at least two oxidants in an alkaline oxidative pretreatment process to produce a catalytically pretreated plant biomass. In one embodiment, the oxidants are oxygen and hydrogen peroxide. In one embodiment, the oxygen pressure is from about 25 psi to about 100 psi and the hydrogen peroxide is less than about 5% (w/w). In one embodiment, the at least two oxidants are present simultaneously in the pretreatment.

In one embodiment, the oxidative pretreatment is conducted at a temperature from about 70° C. to about 90° C. In one embodiment, the method comprises an alkaline pre-extraction prior to the oxidative pretreatment process. In one embodiment, the alkaline pre-extraction is conducted at a temperature from about 100° C. to about 140° C.

In one embodiment, the metal-ligand complex is a copper 2,2′-bipyridine complex (Cu(bpy)). In one embodiment, the metal-ligand complex comprises a second ligand.

In one embodiment, the method further comprises extracting the lignocellulosic biomass prior to produce a solids fraction and a liquid fraction, wherein the solids fraction is catalytically pretreated. In one embodiment, the catalytic pretreating step also produces a liquid phase and the method further comprises: separating the catalytically pretreated biomass from the liquid phase to produce separated catalytically pretreated biomass; and hydrolyzing the separated catalytically pretreated biomass to produce hydrolyzed catalytically pretreated biomass. In one embodiment, the hydrolyzing is conducted with an enzyme, wherein the enzyme is at a concentration from about 5 mg protein/g glucan to about 30 mg protein/g glucan.

The various embodiments further include a method of reducing cost in a homogenous catalytic reaction comprising catalytically pretreating plant biomass with a metal-ligand complex and at least two oxidants present in an alkaline oxidative pretreatment process to produce a catalytically pretreated plant biomass and hydrolyzing the catalytically pretreated biomass to produce hydrolyzed catalytically pretreated biomass for use as a biofuel.

In one embodiment, the oxidants are oxygen and hydrogen peroxide, wherein the oxygen pressure is from about 25 psi to about 100 psi and the hydrogen peroxide is about 5% (w/w) or less. In one embodiment, the metal-ligand complex is a copper 2,2′-bipyridine complex (Cu(bpy)).

In one embodiment, the method further comprises an alkaline pre-extraction step prior to the oxidative pretreatment process and wherein the alkaline pre-extraction is conducted at a temperature from about 100° C. to about 140° C. and wherein the oxidative pretreatment is conducted at a temperature from about 70° C. to about 90° C.

In one embodiment, the catalytic pretreating step produces a liquid phase and the method further comprises separating the catalytically pretreated biomass from the liquid phase.

The various embodiments further include a homogeneous catalyst (e.g., copper (II) 2,2′-bipyridine ethylenediamine (Cu(bpy)en) comprising one or more metals; and at least two metal coordinating ligands, wherein the homogeneous catalyst is a multi-ligand metal complex adapted for use with an oxidant (e.g., air, oxygen, hydrogen peroxide, persulfate, percarbonate and sodium peroxide and/or ozone) in an oxidation reaction to catalytically pretreat lignocellulosic biomass. In one embodiment, the multi-ligand metal complex is a multi-ligand copper complex. In one embodiment, the metals are selected from aluminum, zinc, nickel, magnesium and combinations thereof. In one embodiment, said metals and said metal coordinating ligands are in a state of interaction with each other.

In various embodiments, the one or more metals are metal ions capable of oxidation and/or reduction. In one embodiment, said metals are selected from Fe(II), Fe(III)), Cu(I), Cu(II), Co(III), Co(VI)), V(II), V(III), V(IV), V(V) and combinations thereof.

In one embodiment, the metal coordinating ligand is selected from pyridine, 1,10-phenanthroline, ethylenediamene, histidine, glycine and combinations thereof.

In one embodiment, the lignocellulosic biomass contains more than trace amounts of at least one transition metal, such as, but not limited to, iron, copper and/or manganese.

In one embodiment, a method of pretreating plant biomass is provided, comprising catalytically pretreating the plant biomass with a multi-ligand metal complex and oxidant in an alkaline oxidative pretreatment to produce a catalytically pretreated plant biomass.

In one embodiment, the plant biomass, the multi-ligand metal complex and the oxidant form a solution having a pH of at least 11.5. In one embodiment, the oxidant is hydrogen peroxide and the multi-ligand metal complex is a multi-ligand copper complex.

In one embodiment, the copper complex is a copper(II) 2,2′-bipyridine complex (Cu(bpy)) modified to contain at least one additional metal-coordinating ligand, such as ethylenediamine.

In one embodiment, the oxidant is added at a gradual rate. In one embodiment, the gradual rate is equal to or less than a rate of consumption of the oxidant by the plant biomass and the multi-ligand metal complex.

In various embodiments, the method can further comprise extracting the lignocellulosic biomass prior to produce a solids fraction and a liquid fraction, wherein the solids fraction is catalytically pretreated and/or recovering and reusing the multi-ligand metal complex.

In one embodiment, the catalytic pretreating step also produces a liquid phase and the method further comprises separating the catalytically pretreated biomass from the liquid phase to produce separated catalytically pretreated biomass; and hydrolyzing the separated catalytically pretreated biomass to produce hydrolyzed catalytically pretreated biomass.

All publications, patents and patent documents are incorporated by reference herein, as though individually incorporated by reference, each in their entirety, as though individually incorporated by reference. In the case of any inconsistencies, the present disclosure, including any definitions therein, will prevail.

Although specific embodiments have been illustrated and described herein, it will be appreciated by those of ordinary skill in the art that any procedure that is calculated to achieve the same purpose may be substituted for the specific embodiments shown. For example, although the process has been discussed using particular types of plant biomass, any type of plant biomass, such as grasses, rice straw and the like, for example, may be used. Additionally, although the process has been discussed using primarily copper as the metal in the multi-ligand metal catalyst, other metals, such as iron, in various oxidation states, for example, may be used. This application is intended to cover any adaptations or variations of the present subject matter. Therefore, it is manifestly intended that embodiments of this invention be limited only by the claims and the equivalents thereof.

What is claimed is:

1. A method of pretreating plant biomass comprising catalytically pretreating the plant biomass with a metal-ligand complex and at least two oxidants in an alkaline oxidative pretreatment process to produce a catalytically pretreated plant biomass.

2. The method of claim 1, wherein the oxidants are oxygen and hydrogen peroxide.

3. The method of claim 2, wherein the oxygen pressure is from about 25 psi to about 100 psi and the hydrogen peroxide is less than about 5% (w/w).

4. The method of claim 1, wherein the at least two oxidants are present simultaneously in the pretreatment.

5. The method of claim 1, wherein the oxidative pretreatment is conducted at a temperature from about 70° C. to about 90° C.

6. The method of claim 1, wherein the method comprises an alkaline pre-extraction step prior to the oxidative pretreatment process and wherein the alkaline pre-extraction step is conducted at a temperature from about 100° C. to about 140° C.

7. The method of claim 1, wherein the metal-ligand complex is a copper 2,2′-bipyridine complex (Cu(bpy)).

8. The method of claim 1, wherein the metal-ligand complex comprises a second ligand.

9. The method of claim 1, wherein the catalytic pretreating step produces a liquid phase and the method further comprises:
separating the catalytically pretreated biomass from the liquid phase to produce separated catalytically pretreated biomass; and
hydrolyzing the separated catalytically pretreated biomass to produce hydrolyzed catalytically pretreated biomass.

10. The method of claim 9, wherein the hydrolyzing is conducted with an enzyme, wherein the enzyme is at a concentration from about 5 mg protein/g glucan to about 30 mg protein/g glucan.

11. A homogeneous catalyst system comprising:
one or more metals;
at least one metal coordinating ligand; and
at least two oxidants, wherein the homogeneous catalyst system is a metal-ligand complex configured to catalytically delignify alkaline pretreated lignocellulosic biomass with the at least two oxidants in an oxidative pretreatment reaction.

12. The homogeneous catalyst system of claim 11, wherein the oxidants are selected from air, oxygen, hydrogen peroxide, persulfate, percarbonate, sodium peroxide, ozone and combinations thereof.

13. The homogeneous catalyst system of claim 11, wherein the oxidants are oxygen and hydrogen peroxide.

14. The homogeneous catalyst system of claim 13, wherein the oxygen pressure is from about 25 psi to about 100 psi and the hydrogen peroxide is less than about 5% (w/w).

15. The homogenous catalyst system of claim 11, wherein the one or more metals are metal ions capable of oxidation and/or reduction.

16. The homogenous catalyst system of claim 15, wherein the metal ions are selected from Fe(II), Fe(III)), Cu(I), Cu(II), Co(III), Co(VI)), V(II), V(III), V(IV), V(V) and combinations thereof.

17. The homogeneous catalyst system of claim 11, wherein the metal-ligand complex is a copper-ligand complex.

18. The homogeneous catalyst system of claim 11, wherein the metal coordinating ligand is selected from pyridine, 1,10-phenanthroline, ethylenediamene, histidine, glycine and combinations thereof.

19. The homogeneous catalyst system of claim 11, wherein the metal-ligand complex is copper 2,2′-bipyridine (Cu(bpy)).

20. A method of reducing cost in a homogenous catalytic reaction comprising catalytically pretreating plant biomass with a metal-ligand complex and at least two oxidants present in an alkaline oxidative pretreatment process to produce a catalytically pretreated plant biomass, hydrolyzing the catalytically pretreated biomass to produce hydrolyzed catalytically pretreated biomass for use as a biofuel.

21. The method of claim 20, wherein the oxidants are oxygen and hydrogen peroxide, wherein the oxygen pressure is from about 25 psi to about 100 psi and the hydrogen peroxide is about 5% (w/w) or less.

22. The method of claim 20, wherein the metal-ligand complex is a copper 2,2′-bipyridine complex (Cu(bpy)).

23. The method of claim 20, wherein the method comprises an alkaline pre-extraction prior to the oxidative pretreatment process and wherein the alkaline pre-extraction is conducted at a temperature from about 100° C. to about 140° C. and wherein the oxidative pretreatment is conducted at a temperature from about 70° C. to about 90° C.

24. The method of claim 20, wherein the catalytic pretreating step produces a liquid phase and the method further comprises separating the catalytically pretreated biomass from the liquid phase to produce separated catalytically pretreated biomass.

25. The method of claim 20, wherein the method reduces the minimum fuel selling price (MFSP) by about 40% relative to the MFSP of a method using only hydrogen peroxide as the oxidant.
